(19) United States
(12) Patent Application Publication
(10) Pub. No.: US 2008/0255078 A1
(43) Pub. Date: Oct. 16, 2008

(54) Triphenylethylene Compounds Useful as Selective Estrogen Receptor Modulators

(76) Inventor: Subba Reddy Katamreddy, Durham, NC (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/094,190

(22) PCT Filed: Nov. 22, 2006
(86) PCT No.: PCT/US2006/045250

§ 371 Date: May 19, 2008

Related U.S. Application Data
(60) Provisional application No. 60/738,918, filed on Nov. 22, 2005.

(51) Int. Cl. A61K 31/565; C07C 57/42; A61K 31/192; A61K 31/42; A61P 3/10; A61P 9/10; C07D 261/08; A61K 31/09; C07C 43/23

(52) U.S. Cl. 514/182; 562/468; 514/570; 514/378; 548/247; 514/571; 568/640; 514/720

(57) ABSTRACT

Triphenylethylene compounds of formula (I) are provided. The compounds are particularly useful for selective estrogen receptor modulation.

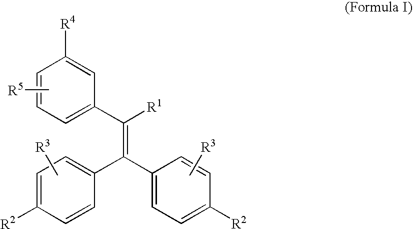

### FIELD OF THE INVENTION

The present invention relates to novel compounds with a variety of therapeutic uses, more particularly to symmetrical triphenyl compounds that are particularly useful for selective estrogen receptor modulation (SERM).

### BACKGROUND OF THE INVENTION

Estrogens are well-known endocrine regulators in the cellular processes involved in the development and maintenance of the reproductive system. Estrogens have also been shown to have important effects in many non-reproductive tissues such as bone, liver, the cardiovascular system, and the central nervous system. The most widely accepted hypothesis of how estrogens exert their effects is by binding to an intracellular steroid hormone receptor. After the receptor and bound ligand are transferred to the nucleus of the cell, the complex binds to recognition sites in DNA, which allows for the modulation of certain genes. Additionally, it is now becoming apparent that estrogens may mediate their effects via membrane-initiated signaling cascade, though much of this work is still experimental. Kousteni et al., Journal of Clinical Investigation, (2003), 111, 1651-1664, herein incorporated by reference with regard to such teaching.

Certain substances have demonstrated the ability to exhibit their biological activity in a “tissue-selective” manner. In other words, tissue selectivity allows functionality as estrogen agonists in certain tissues, while acting as estrogen antagonists in other tissues. The term “selective estrogen receptor modulators” (SERMs) has been given to these molecules. Examples of SERMs include tamoxifen, raloxifene, lasofoxifene, clomiphene, and nafoxidine. The molecular basis for this tissue-selective activity is not completely understood. Without being limited to any particular theory, the ability of the ligand to place the estrogen receptor into different conformational states and allowing for differential capabilities in recruiting coactivator and corepressor proteins, as well as other important proteins involved in transcriptional regulation, is believed to play a role. See, McDonnell, D. P., The Molecular Pharmacology of SERMs, Trends Endocrinol. Metab. 1999, 301-311, herein incorporated by reference with regard to such description.

Historically estrogens were believed to manifest their biological activity through a single estrogen receptor, now termed estrogen receptor alpha (ERα). More recently, however, there was the discovery of second subtype of estrogen receptor, termed estrogen receptor beta (ERβ). See, Kuiper et al., WO 97/09348 and Kuiper et al., Cloning of a Novel Estrogen Receptor Expressed in Rat Prostate and Ovary, Proc. Natl. Acad. Sci. U.S.A., 1996, pp. 5925-5930, herein incorporated by reference with regard to such subtype. ERβ is expressed in humans. See, Mosselman et al., ERβ: Identification and Characterization of a Novel Human Estrogen Receptor, FEBR S Lett., 1996, pp. 49-53, herein incorporated by reference with regard to such expression. The discovery of this second subtype of estrogen receptor significantly increased the biological complexity of estrogen signaling and may be responsible for some of the tissue-selective actions of the currently available SERMs.

As noted above, estrogens have important effects in many non-reproductive tissues. Thus, estrogen modulation is believed useful in the treatment or prophylaxis of diseases and conditions associated with such tissues, including bone, liver, and the central nervous system. For example, osteoporosis is characterized by the net loss of bone mass per unit volume. Such bone loss results in a failure of the skeleton to provide adequate structural support for the body, thereby creating an increased risk of fracture. One of the most common types of osteoporosis is postmenopausal osteoporosis, which is associated with accelerated bone loss subsequent to cessation of menses and declining levels of endogenous estrogen in women. There is an inverse relationship between densitometric measures of bone mass and fracture risk, for peri- and postmenopausal women in the process of rapid bone loss due to declining levels of estrogen. See, Slemenda, et al., Predictors of Bone Mass in Perimenopausal Women, A Prospective Study of Clinical Data Using Photon Abr sorptiometry, Ann. Intern. Med., 1990, pp. 96-101 and Marshall, et al., Meta-Analysis of How Well Measures of Bone Mineral Density Predict Occurrence of Osteoporotic Fractures, Br Med. J., 1996, pp. 1254-1259, each of which is herein incorporated by reference with regard to such relationship. Elderly women currently have a lifetime risk of fractures of about 75%. In addition there is an approximate 40% risk of hip fracture for Caucasian women over age 50 in the United States. The economic burden from osteoporotic fractures is considerable because of the necessity of hospitalization. In addition, although osteoporosis is generally not thought of as life-threatening, the mortality within 4 months of hip fracture is currently approximately 20 to 30%. Current therapies for postmenopausal osteoporosis include hormone replacement therapy or treatment with other antiresorptive agents such as bisphosphonates or calcitonin. Similarly, SERMS have been shown to be effective in the treatment of postmenopausal osteoporosis (see, Lindsay, R.: Sex steroids in the pathogenesis and prevention of osteoporosis. In: Osteoporosis 1988. Etiology, Diagnosis and Management. Riggs B L (ed)I, Raven Press, New York, USA (1988):333-358; Barzel U S: Estrogens in the prevention and treatment of postmenopausal osteoporosis: a review. Am J. Med (1988) 85:847-850; and Ettinger, B., Black, D. M., et al., Reduction of Vertebral Fracture Risk in Postmenopausal Women with Osteoporosis Treated with Raloxifene, JAMA, 1999, 282, 637645, each of which is incorporated by reference with regard to such teaching).

As another example, the effects of estrogens on breast tissue, particularly breast cancer, have been well documented. For example, a previously identified SERM, tamoxifen, decreases the risk of recurrent breast cancer, contralateral breast cancer, and mortality as well as increases the disease-free survival rate of patients with breast cancer at multiple stages of the disease. See, Cosman, F., Lindsay, R. Selective Estrogen Receptor Modulators: Clinical Spectrum, Endocrine Rev., 1999, pp. 418-434, herein incorporated by reference with regard to such teaching. The profile of tamoxifen, however, is not ideal due to potential interactive properties on reproductive tissues, such as uterine tissues. There is room for an improved therapy for the treatment of such cancers, namely a SERM with reduced agonist properties on reproductive tissues.

Cardiovascular disease is the leading cause of death among postmenopausal women. Until recently, the preponderance of data suggested that estrogen replacement therapy in postmenopausal women reduced the risk of cardiovascular disease, although some studies reported no beneficial effect on overall mortality. See, Barrett-Connor, E. et al., The Potential of SERMs for Reducing the Risk of Coronary Heart Disease, Trends Endocrinol. Metab., 1999, pp. 320-325, herein incorporated by reference. The mechanism(s) by which estrogens were believed to exert their beneficial effects on the cardiovascular system are not entirely clear. Potentially estrogen's effects on serum cholesterol and lipoproteins, antioxidant properties, vascular smooth muscle proliferation, and inhibition of arterial cholesterol accumulation were believed to play a role. Id. See also, Cosman, F., Lindsay, R. Selective Estrogen Receptor Modulators: Clinical Spectrum, Endocrine Rev., 1999, pp. 418-434, herein incorporated by reference. In light of the recent reports of the HERS II and WHI studies, however, continuous combined Hormone Therapy, namely, CEE+MPA [Conjugated Equine Estrogen+Medroxy Progesterone Acetate], confers no cardiovascular benefit in menopausal women. See, Hulley S., Grady, D., Bush, T., et al., Randomized trial of estrogen plus progestin for secondary prevention of coronary heart disease in postmenopausal women. Heart and Estrogen/progestin Replacement Study (HERS) Research Group. J. Am. Med. Assoc. (1998) 280:605-613 and Wassertheil-Smoller S., Hendrix, S. L., Limacher, M., et al., for the WHI Investigators. Effect of estrogen plus progestin on stroke in postmenopausal women: the Women's Health Initiative: a randomized trial JAMA (2003) 289, 2673-2684, each herein incorporated by reference with regard to such teaching). To what extent these findings may be extrapolated to SERMs is an issue that remains to be determined.

Other therapeutic alternatives include estrogen replacement therapy and/or hormone replacement therapy, which may be useful in the treatment of vasomotor symptoms, genitourinary atrophy, depression, and diabetes. Over 75% of women experience vasomotor symptoms during the climacteric years. Clinical signs, such as vasomotor symptoms and genitourinary atrophy, abate upon treatment with estrogen replacement therapy. Sagraves, R., J. Clin. Pharmacol. (1995), 35(9 Suppl):2S-10S, herein incorporated by reference with regard to such teaching. Preliminary data suggest that estradiol may alleviate depression during perimenopause and that the combination of estrogens and selective serotonin reuptake inhibitors may alleviate depression during the postmenopausal period. Soares, C. N., Poitras, J. R., and Prouty, J., Drugs Aging, (2003), 20(2), 85-100, herein incorporated by reference with regard to such teaching. Furthermore, hormone replacement therapy may improve glycemic control among women with diabetes. Palin, S. L. et al., Diabetes Research and Clinical Practice, (2001), 54, 67-77; Ferrara, A. et al., Diabetes Care, (2001), 24(7), 1144-1150), each incorporated herein by reference with regard to such teaching. There is a need, however, for improved therapies that present better side effect profiles.

### SUMMARY OF THE INVENTION

The present inventors discovered a novel group of symmetrical triphenyl compounds, which bind to and modulate estrogen receptor alpha and estrogen receptor beta. As SERMS, these compounds are believed to be useful for the treatment and/or prophylaxis of conditions such as menopausal or postmenopausal disorders, vasomotor symptoms, urogenital or vulvar vaginal atrophy, atrophic vaginitis, female sexual dysfunction, breast cancer, depressive symptoms, diabetes, bone demineralization, and the treatment and/or prevention of osteoporosis.

The present invention provides a compound of Formula I

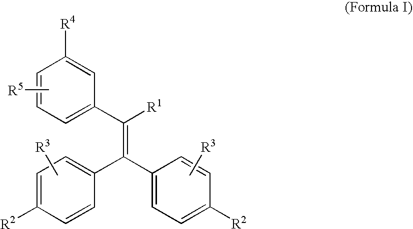

or a pharmaceutically acceptable salt or solvate, or physiologically thereof, wherein
R¹ is selected from the group consisting of C₁-C₆ alkyl and C₁-C₆ haloalkyl;
each R² is the same and selected from the group consisting of —OH, halogen, C₁-C₄ alkoxy, —S—R⁸, —SO—R³, —SO₂—R⁸, and —C≡N;
each R³ is the same and selected from hydrogen, —OH, C₁-C₆ alkyl, halogen, C₁-C₆ alkoxy, and C₁-C₆ haloalkyl;
R⁹ is independently selected from H and substituted or unsubstituted C₁-C₆ alkyl group;
R¹¹ and R¹² are independently selected from H, R^{a}H, R^{a}OH, and R^{a}OMe;
R^{a} and R^{b} are independently selected from C₁-C₆ alkylene,
and,
R⁴ is selected from —COOH, —C≡N, —C≡C—R^{a}—OH, —C≡C—COO—R⁸, —C═NOH, —C═C—R⁶, —O—R^{a}—R¹⁰, —N—R¹¹R¹², —N—SO₂—R⁸, —SOR⁸, —SO₂—R⁸, and a saturated or unsaturated heterocycle containing 1 or 2 heteroatoms selected from N and O optionally substituted with one or more of C₁-C₆ alkyl, phenyl, and C₁-C₆ alkylphenyl;
R⁵ is selected from hydrogen, —OH, C₁-C₆ alkyl, halogen, C₁-C₆ alkoxy, and C₁-C₆ haloalkyl;
R⁶ is selected from R^{a}—COOH, COO—R⁸, —CONR⁸R⁹;
R⁸ is independently selected from H and substituted or unsubstituted C₁-C₆ alkyl group; and
R¹⁰ is selected from COOH, OH, O—R^{b}—OH, O—R^{b}—OCO—R⁸, and COO—R⁸;
or,
R⁴ is selected from —COOH, —C≡N, —C═C—R⁶, —O—R^{a}—R¹⁰, —SOR⁸, —SO₂—R⁸, and a saturated or unsaturated heterocycle containing 1 or 2 heteroatoms selected from N and O optionally substituted with one or more of C₁-C₆ alkyl, phenyl, and C₁-C₆ alkylphenyl;
R⁵ is selected from hydrogen, —OH, C₁-C₆ alkyl, halogen, C₁-C₆ alkoxy, and C₁-C₆ haloalkyl;
R⁶ is selected from —COOH, R^{a}—COOH, COO—R⁸, —CONR⁸R⁹;
R⁸ is selected from substituted or unsubstituted C₁-C₆ alkyl group; and
R¹⁰ is selected from COOH, OH, O—R^{b}—OH, O—R^{b}—OCO—R⁸, and COO—R⁸;
or,
R⁴ is selected from —COOH, —C≡N, —C═C—R⁶, —O—R^{a}—R¹⁰, —SOR⁸, —SO₂—R^{a}, and a saturated or unsaturated heterocycle containing 1 or 2 heteroatoms selected from N and O optionally substituted with one or more of C₁-C₆ alkyl, phenyl, and C₁-C₆ alkylphenyl;
R⁵ is selected from hydrogen, —OH, halogen, and C₁-C₆ alkoxy;
R⁶ is selected from —COOH, R^{a}—COOH, COO—R⁸;
R⁸ is an unsubstituted C₁-C₆ alkyl group; and
R¹⁰ is selected from COOH, OH, NH₂, NHR⁸, O—R^{b}—OH, O—R^{b}—OCO—R⁸, and COO—R⁸.

According to another embodiment, the invention provides a compound of Formula I, a salt, or a solvate, thereof for use as an active therapeutic substance.

According to another embodiment, the invention provides a pharmaceutical composition comprising compound of Formula I, a salt, or a solvate thereof and a pharmaceutically acceptable carrier.

According to another embodiment, the invention provides a compound of Formula I, a salt, or a solvate thereof for use in the treatment, including phophylaxis, of a condition or disorder affected by selective estrogen receptor modulation.

According to another embodiment, the invention provides the use of a compound of Formula I, or a salt, or a solvate thereof for use in the treatment, which may include phophylaxis, of a condition or disorder affected by selective estrogen receptor modulation.

According to another embodiment, the invention provides the use of a compound of formula I, or a salt, or a solvate thereof in the manufacture of a medicament for use in the treatment, which is used hereinafter to include prophylaxis, of a condition or disorder affected by selective estrogen receptor modulation.

According to another embodiment, the invention provides a method of treatment, which may include phophylaxis, of a condition or disorder affected by selective estrogen receptor modulation in a mammal in need thereof, with a compound of Formula I, or a salt, or a solvate thereof.

According to another embodiment, the present invention provides a method for treating conditions such as those selected from menopausal or postmenopausal disorders, vasomotor symptoms, urogenital or vulvar vaginal atrophy, atrophic vaginitis, endometriosis, female sexual dysfunction, breast cancer, depressive symptoms, diabetes, bone demineralization, and osteoporosis

### DETAILED DESCRIPTION OF THE INVENTION

The invention herein is described in terms known and appreciated by those skilled in the art. For ease of reference certain terms are defined. The fact that certain terms are defined, however, should not be considered as indicative that any term that is undefined is indefinite. Rather, all terms used are believed to describe the invention in terms such that one of ordinary skill can appreciate the scope and practice the present invention.

As used herein the term “alkyl” refers to a straight or branched chain hydrocarbon having from one to twelve carbon atoms. Examples of “alkyl” as used herein include, but are not limited to, methyl, ethyl, propyl, isopropyl, isobutyl, n-butyl, tert-butyl, isopentyl, n-pentyl, and the like.

As used herein, the term “alkylene” refers to a straight or branched chain divalent hydrocarbon radical having from one to ten carbon atoms. Examples of “alkylene” as used herein include, but are not limited to, methylene, ethylene, n-propylene, n-butylene, and the like.

As used herein the term “halogen” refers to fluorine, chlorine, bromine, or iodine.

As used herein the term “haloalkyl” refers to an alkyl group, as defined herein, which is substituted with at least one halogen. Examples of branched or straight chained “haloalkyl” groups useful in the present invention include, but are not limited to, methyl, ethyl, propyl, isopropyl, n-butyl, and t-butyl substituted independently with one or more halogens, for example, fluoro, chloro, bromo, and iodo. The term “haloalkyl” should be interpreted to include such substituents as perfluoroalkyl groups (i.e., trifluoromethyl) and the like.

As used herein the term “alkoxy” refers to the group —OR, where R is alkyl as defined above.

As used herein the term “acyl” refers to the group —C(O)R, where R is alkyl, aryl, heteroaryl, or heterocyclyl, as each is defined herein.

As used herein the term “hydroxy” refers to the group —OH.

As used herein the term “carboxy” refers to the group —C(O)OH.

As used herein the term “nitro” refers to the group —NO₂.

As used herein the term “amino” refers to the group —NH₂, or when referred to as substituted amino defines such groups substituted with alkyl.

As used herein, the term “cycloalkyl” refers to a non-aromatic, saturated or unsaturated, mono- or bi-cyclic hydrocarbon ring having from three to ten carbon atoms. Exemplary “cycloalkyl” groups include, but are not limited to, cyclopropyl, cyclobutyl, cyclopentyl, cyclohexyl, and cycloheptyl.

As used herein, the term “aryl” refers to a phenyl ring or to a phenyl ring system fused to one or more additional phenyl rings to form, for example, anthracene, phenanthrene, or naphthalene ring systems. Examples of “aryl” groups include, but are not limited to, phenyl, 2-naphthyl, 1-naphthyl, biphenyl, and the like.

As used herein, the term “heteroaryl” refers to a monocyclic five to seven membered aromatic ring, or fused bicyclic aromatic ring system comprising two of such monocyclic five to seven membered aromatic rings. These heteroaryl rings contain one to four heteroatoms selected from N, O, and S, where N-oxides, sulfur oxides, and dioxides are permissible heteroatom substitutions. Examples of “heteroaryl” groups used herein include, but should not be limited to, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, indazole, and the like.

As used herein, the term “heterocycle” or “heterocyclyl” refers to a non-aromatic, mono- or bi-cyclic ring system containing optionally one or more degrees of unsaturation and also containing one to four heteroatoms selected from N, O and/or S. “Heterocycle” and “heterocyclyl” also includes variants thereof wherein the heteroatom, N or S is substituted by oxo to provide N-oxides and sulfur oxide. Preferred heteroatoms include N, O, or both. Preferably the ring is three to ten-membered and is either saturated or has one or more degrees of unsaturation. Such rings may be optionally fused to one or more of another “heterocyclic” ring(s), heteroaryl ring(s), aryl ring(s), or cycloalkyl ring(s). Examples of “heterocyclic” groups include, but are not limited to, tetrahydrofuran, pyran, 1,4-dioxane, 1,3-dioxane, piperidine, pyrrolidine, morpholine, tetrahydrothiopyran, and tetrahydrothiophene.

Typically, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts. Representative salts include acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, sulfate, tannate, tartrate, teoclate, tosylate, triethiodide, trimethylammonium, and valerate salts. Other salts, which are not pharmaceutically acceptable, may be useful in the preparation of compounds of this invention and these should be considered to form a further aspect of the invention.

As used herein, the term “solvate” refers to a complex of variable stoichiometry formed by a solute (in this invention, a compound of Formula I, or a salt or physiologically functional derivative thereof) and a solvent. Such solvents, for the purpose of the invention, should not interfere with the biological activity of the solute. Non-limiting examples of suitable solvents include, but are not limited to water, methanol, ethanol, and acetic acid. Preferably the solvent used is a pharmaceutically acceptable solvent. Non-limiting examples of suitable pharmaceutically acceptable solvents include water, ethanol, and acetic acid. Most preferably the solvent used is water.

As used herein, a substituent may be indicated as attached to a ring structure using the following representation:

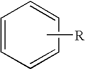

This representation indicates that the R substituent may be located at any point on the ring structure that is not otherwise occupied by specifically specified substituents or groups.

The present invention provides a compound of Formula I

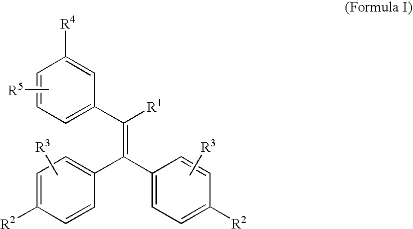

or a pharmaceutically acceptable salt or solvate, or physiologically thereof, wherein
R¹ is selected from the group consisting of C₁-C₆ alkyl and C₁-C₆ haloalkyl;
each R² is the same and selected from the group consisting of —OH, halogen, C₁-C₄ alkoxy, —S—R⁸, —SO—R⁸, —SO₂—R⁸, and —C≡N;
each R³ is the same and selected from hydrogen, —OH, C₁-C₆ alkyl, halogen, C₁-C₆ alkoxy, and C₁-C₆ haloalkyl;
R⁹ is independently selected from H and substituted or unsubstituted C₁-C₆ alkyl group;
R¹¹ and R¹² are independently selected from H, R^{a}H, R^{a}OH, and R^{a}OMe;
R^{a} and R^{b} are independently selected from C₁-C₆ alkylene,
and,
R⁴ is selected from —COOH, —C≡N, —C≡C—R^{a}—OH, —C≡C—COO—R⁸, —C═NOH, —C═C—R⁶, —O—R^{a}—R¹⁰, —N—R¹¹R¹², —N—SO₂—R⁸, —SOR⁸, —SO₂—R⁸, and a saturated or unsaturated heterocycle containing 1 or 2 heteroatoms selected from N and O optionally substituted with one or more of C₁-C₆ alkyl, phenyl, and C₁-C₆ alkylphenyl;
R⁵ is selected from hydrogen, —OH, C₁-C₆ alkyl, halogen, C₁-C₆ alkoxy, and C₁-C₆ haloalkyl;
R⁶ is selected from R^{a}—COOH, COO—R⁸, —CONR⁸R⁹;
R⁸ is independently selected from H and substituted or unsubstituted C₁-C₆ alkyl group; and
R¹⁰ is selected from COOH, OH, O—R^{b}—OH, O—R^{b}—OCO—R⁸, and COO—R⁸;
or,
R⁴ is selected from —COOH, —C≡N, —C═C—R⁶, —O—R^{a}—R¹⁰, —SOR⁸″-SO₂—R⁸, and a saturated or unsaturated heterocycle containing 1 or 2 heteroatoms selected from N and O optionally substituted with one or more of C₁-C₆ alkyl, phenyl, and C₁-C₆ alkylphenyl;
R⁵ is selected from hydrogen, —OH, C₁-C₆ alkyl, halogen, C₁-C₆ alkoxy, and C₁-C₆ haloalkyl;
R⁶ is selected from —COOH, R^{a}—COOH, COO—R⁸, —CONR⁸R⁹;
R⁸ is selected from substituted or unsubstituted C₁-C₆ alkyl group; and
R¹⁰ is selected from COOH, OH, O—R^{b}—OH, O—R^{b}—OCO—R⁸, and COO—R⁸;
or,
R⁴ is selected from —COOH, —C≡N, —C═C—R⁶, —O—R^{a}—R¹⁰, —SOR⁸, —SO₂—R⁸, and a saturated or unsaturated heterocycle containing 1 or 2 heteroatoms selected from N and O optionally substituted with one or more of C₁-C₆ alkyl, phenyl, and C₁-C₆ alkylphenyl;
R⁵ is selected from hydrogen, —OH, halogen, and C₁-C₆ alkoxy;
R⁶ is selected from —COOH, R^{a}—COOH, COO—R⁸;
R⁸ is an unsubstituted C₁-C₆ alkyl group; and
R¹⁰ is selected from COOH, OH, NH₂, NHR⁸, O—R^{b}—OH, O—R^{b}—OCO—R⁸, and COO—R⁸.

According to another embodiment, the substituents are as follows:

R¹ is selected from the group consisting of C₁-C₆ alkyl and C₁-C₆ haloalkyl;
each R² is the same and selected from the group consisting of —OH, halogen, C₁-C₄ alkoxy, —S—R⁸, —SO—R⁸, —SO₂—R⁸, and —C≡N;
each R³ is the same and selected from hydrogen, —OH, C₁-C₆ alkyl, halogen, C₁-C₆ alkoxy, and C₁-C₆ haloalkyl;
R⁴ is selected from —COOH, —C≡N, —C≡C—R^{a}—OH, —C≡C—COO—R⁸, —C═NOH, —C═C—R⁶, —O—R^{a}—R¹⁰, —N—R¹¹R¹², —N—SO₂—R⁸, —SO₂—R⁸, and a saturated or unsaturated heterocycle containing 1 or 2 heteroatoms selected from N and O optionally substituted with one or more of C₁-C₆ alkyl, phenyl, and C₁-C₆ alkylphenyl;
R⁵ is selected from hydrogen, —OH, C₁-C₆ alkyl, halogen, C₁-C₆ alkoxy, and C₁-C₆ haloalkyl;
R⁶ is selected from R^{a}—COOH, COO—R⁸, —CONR⁸R⁹;
R⁸ and R⁹ are independently selected from H and substituted or unsubstituted C₁-C₆ alkyl group; and R¹⁰ is selected from COOH, OH, O—R^{b}—OH, O—R^{b}—OCO—R⁸, and COO—R⁸; R¹¹ and
R¹² are independently selected from H, R^{a}H, R^{a}OH, and R^{a}OMe; and R^{a} and R^{b} are independently selected from C₁-C₆ alkylene.

According to another embodiment, R¹ is advantageously a C₁₋₆ alkyl and more advantageously R¹ is ethyl.

According to another embodiment, R³ is advantageously hydrogen.

According to another embodiment, R² is advantageously —OH.

According to another embodiment, R⁴ is advantageously selected from —C═C—COOH, —COOH, and —O(CH₂)₂O(CH₂)₂OH.

According to another embodiment, R⁵ is advantageously positioned in a para orientation.

According to another embodiment, the R³ substituents are advantageously positioned in a meta orientation.

According to another embodiment, the substituents are as follows: R¹ is selected from the group consisting of C₁-C₆ alkyl and C₁-C₆ haloalkyl; each R² is the same and selected from the group consisting of —OH, halogen, C₁-C₄ alkoxy, —S—R⁸, —SO—R⁸, —SO₂—R⁸, and —C≡N; each R³ is the same and selected from hydrogen, —OH, C₁-C₆ alkyl, halogen, C₁-C₆ alkoxy, and C₁-C₆ haloalkyl; R⁴ is selected from —COOH, —C≡N, —C═C—R⁶, —O—R^{a}—R¹⁰, —SO₂—R⁸, and a saturated or unsaturated heterocycle containing 1 or 2 heteroatoms selected from N and O optionally substituted with one or more of C₁-C₆ alkyl, phenyl, and C₁-C₆ alkylphenyl; R⁵ is selected from hydrogen, —OH, C₁-C₆ alkyl, halogen, C₁-C₆ alkoxy, and C₁-C₆ haloalkyl; R⁶ is selected from —COOH, R^{a}—COOH, COO—R⁸, —CONR⁸R⁹; R^{a} and R^{b} are independently selected from C₁-C₆ alkylene; R¹ is selected from substituted or unsubstituted C₁-C₆ alkyl group; and R¹⁰ is selected from COOH, OH, O—R^{b}—OH, O—R^{b}—OCO—R⁸, and COO—R⁸.

According to another embodiment, the substituents are as follows: R¹ is selected from the group consisting of C₁-C₆ alkyl and C₁-C₆ haloalkyl; each R² is the same and selected from the group consisting of —OH, halogen, C₁-C₄ alkoxy, —S—R⁸, —SO—R⁸, —SO₂—R⁸, and —C≡N; each R³ is the same and selected from hydrogen, —OH, C₁-C₆ alkyl, halogen, C₁-C₆ alkoxy, and C₁-C₆ haloalkyl; R⁴ is selected from —COOH, —C≡N, —C═C—R⁶, —O—R^{a}—R¹⁰, —SO₂—R⁸, and a saturated or unsaturated heterocycle containing 1 or 2 heteroatoms selected from N and O optionally substituted with one or more of C₁-C₆ alkyl, phenyl, and C₁-C₆ alkylphenyl; R⁵ is selected from hydrogen, —OH, halogen, and C₁-C₆ alkoxy; R⁶ is selected from —COOH, R^{a}—COOH, COO—R⁸; R^{a} and R^{b} are independently selected from C₁-C₆ alkylene; R³ is an unsubstituted C₁-C₆ alkyl group; and R¹⁰ is selected from COOH, OH, NH₂, NHR⁸, O—R^{b}—OH, O—R^{b}—OCO—R⁸, and COO—R⁸.

It is to be understood that the present invention includes all combinations and subsets of the particular groups defined hereinabove.

Particularly preferred compounds of the present invention include:
(2E)-3-{3-[1-ethyl-2,2-bis(4-hydroxyphenyl)ethenyl]phenyl}-2-propenoic acid;
(2E)-3-{5-[1-Ethyl-2,2-bis(4-hydroxyphenyl)ethenyl]-2-fluorophenyl}-2-propenoic acid;
4,4′-{2-[3-(3,5-Dimethyl-4-isoxazolyl)phenyl]-1-butene-1,1-diyl}diphenol;
4,4′-{2-[3-(3,5-Dimethyl-4-isoxazolyl)-4-fluorophenyl]-1-butene-1,1-diyl}diphenol;
4,4′-{2-[3-(3-Furanyl)1-butene-1,1-diyl}diphenol;
4,4′-{2-[4-Fluoro-3-(3-furanyl)phenyl]-1-butene-1,1-diyl}diphenol;
4,4′-(2-{3-[(2-Hydroxyethyl)oxy]phenyl}-1-butene-1,1-diyl)diphenol;
({3-[1-ethyl-2,2-bis(4-hydroxyphenyl)ethenyl]phenyl}oxy)acetic acid;
4-({3-[1-Ethyl-2,2-bis(4-hydroxyphenyl)ethenyl]phenyl}oxy)butanoic acid;
4,4′-(2-{3-[(4-Hydroxybutyl)oxy]phenyl}-1-butene-1,1-diyl)diphenol;
4,4′-{2-[3-({2-[(2-Hydroxyethyl)oxy]ethyl}oxy)phenyl]-1-butene-1,1-diyl}diphenol;
4,4′-(2-{3-[(2-Hydroxyethyl)oxy]phenyl}-1-pentene-1,1-diyl)diphenol;
4,4′-(2-{3-[(4-hydroxybutyl)oxy]phenyl}-1-pentene-1,1-diyl)diphenol;
4-({3-[2,2-bis(4-Hydroxyphenyl)-1-propylethenyl]phenyl}oxy)butanoic acid;
2-{[2-({3-[2,2-bis(4-hydroxyphenyl)-1-propylethenyl]phenyl}oxy)ethyl]oxy}ethyl acetate
4,4′-{2-[3-({2-[(2-hydroxyethyl)oxy]ethyl}oxy)phenyl]-1-pentene-1,1-diyl}diphenol;
(2E)-3-{3-[1-Ethyl-2,2-bis(4-fluorophenyl)ethenyl]phenyl}-2-propenoic;
(2E)-3-{3-[2,2-bis(4-Cyanophenyl)-1-ethylethenyl]phenyl}-2-propenoic acid;
(2E)-3-{3-[2,2-bis(4-Cyano-3-fluorophenyl)-1-ethylethenyl]phenyl}-2-propenoic acid;
4,4′-{2-[3-({2-[(2-hydroxyethyl)oxy]ethyl}oxy)phenyl]-1-hexene-1,1-diyl}diphenol;
2-{[2-({3-[1-Butyl-2,2-bis(4-fluorophenyl)ethenyl]phenyl}oxy)ethyl]oxy}ethanol;
4,4′-(2-{3-[(2-hydroxyethyl)oxy]phenyl}-1-hexene-1,1-diyl)diphenol;
3-[1-Ethyl-2,2-bis(4-hydroxyphenyl)ethenyl]benzoic acid;
4,4′-{2-[3-[(2-Hydroxyethyl)oxy]-4-(methyloxy)phenyl]-1-butene-1,1-diyl}diphenol; and
4,4′-{2-[3-({2-[(2-hydroxyethyl)oxy]ethyl}oxy)-4-(methyloxy)phenyl]-1-butene-1,1-diyl}diphenol.

Additional compounds, which have not necessarily been fabricated, but which could be fabricated using techniques and reaction schemes described in the Examples, include:
{[5-[1-ethyl-2,2-bis(4-hydroxyphenyl)ethenyl]-2-(methyloxy)phenyl]oxy}acetic acid;
4-{[5-[1-Ethyl-2,2-bis(4-hydroxyphenyl)ethenyl]-2-(methyloxy)phenyl]oxy}butanoic acid;
(2E)-3-[5-[1-Ethyl-2,2-bis(4-hydroxyphenyl)ethenyl]-2-(methyloxy)phenyl]-2-propenoic acid;
4,4′-{2-[3-{[2-(Dimethylamino)ethyl]oxy}-4-(methyloxy)phenyl]-1-butene-1,1-diyl}diphenol;
(2E)-3-{3-[2,2-bis(4-Hydroxyphenyl)-1-propylethenyl]phenyl}-2-propenoic acid;
(2E)-3-{3-[1-Butyl-2,2-bis(4-hydroxyphenyl)ethenyl]phenyl}-2-propenoic acid;
3-[1-Ethyl-2,2-bis(4-hydroxyphenyl)ethenyl]benzonitrile;
5-[(1-Ethyl-2,2-bis(4-hydroxyphenyl)ethenyl]-2-fluorobenzonitrile;
4,4′-{2-[3-(Methylsulfonyl)phenyl]-1-butene-1,1-diyl}diphenol;
4,4′-{2-[4-Fluoro-3-(methylsulfonyl)phenyl]-1-butene-1,1-diyl}diphenol;
2-({2-[(3-{1-Ethyl-2,2-bis[4-(methyloxy)phenyl]ethenyl}phenyl)oxy]ethyl}oxy)ethanol;
2-({2-[(3-{1-Ethyl-2,2-bis[4-(methylthio)phenyl]ethenyl}phenyl)oxy]ethyl}oxy)ethanol;
2-({2-[(3-{1-ethyl-2,2-bis[4-(methylsulfinyl)phenyl]ethenyl}phenyl)oxy]ethyl}oxy)ethanol;
2-({2-[(3-{1-Ethyl-2,2-bis[4-(methylsulfonyl)phenyl]ethenyl}phenyl)oxy]ethyl}oxy)ethanol;
(2E)-3-[5-[2,2-bis(4-hydroxyphenyl)-1-propylethenyl]-2-(methyloxy)phenyl]-2-propenoic acid;
(2E)-3-[5-[1-butyl-2,2-bis(4-hydroxyphenyl)ethenyl]-2-(methyloxy)phenyl]-2-propenoic acid;
(2E)-3-[5-[1-butyl-2,2-bis(4-hydroxyphenyl)ethenyl]-2-(trifluoromethyl)phenyl]-2-propenoic acid;
4,4′-{2-[3-[(2-hydroxyethyl)oxy]-4-(methyloxy)phenyl]-1-pentene-1,1-diyl}diphenol
4,4′-{2-[3-{2-[(2-hydroxyethyl)oxy]ethyl}-4-(methyloxy)phenyl]-1-pentene-1,1-diyl}diphenol
4,4′-{2-[3-[(2-hydroxyethyl)oxy]-4-(methyloxy)phenyl]-1-hexene-1,1-diyl}diphenol;
4,4′-{2-[3-({2-[(2-hydroxyethyl)oxy]ethyl}oxy)-4-(methyloxy)phenyl]-1-hexene-1,1-diyl}diphenol;
4-{[4-[2,2-bis(4-hydroxyphenyl)-1-propylethenyl]-2-(methyloxy)phenyl]oxy}butanoic acid;
4-{[5-[1-butyl-2,2-bis(4-hydroxyphenyl)ethenyl]-2-(methyloxy)phenyl]oxy}butanoic acid; and
3-[1-butyl-2,2-bis(4-hydroxyphenyl)ethenyl]benzaldehyde oxime.

According to an embodiment, a compound of formula I is provided as described in any one of the examples.

The compounds of formulas (I) may crystallize in more than one form, a characteristic known as polymorphism, and such polymorphic forms (“polymorphs”) are within the scope of formula (I). Polymorphism generally can occur as a response to changes in temperature, pressure, or both. Polymorphism can also result from variations in the crystallization process. Polymorphs can be distinguished by various physical characteristics known in the art such as x-ray powder diffraction patterns, infra-red spectra, solubility, and melting point.

Certain of the compounds described herein contain one or more chiral centers, or may otherwise be capable of existing as multiple stereoisomers. The scope of the present invention includes mixtures of stereoisomers as well as purified enantiomers or enantiomerically/diastereomerically enriched mixtures. Also included within the scope of the invention are the individual isomers of the compounds represented by formula (I), as well as any wholly or partially equilibrated mixtures thereof. The present invention also includes the individual isomers of the compounds represented by the formulas above as mixtures with isomers thereof in which one or more chiral centers are inverted.

According to another embodiment, at each occurrence, each alkyl, alkoxy, haloalkyl, and alkalene may be optionally substituted. As used herein throughout the present specification, the phrase “optionally substituted” or variations thereof denote an optional substitution, including multiple degrees of substitution, with one or more substituent group. The phrase should not be interpreted so as to be imprecise or duplicative of substitution patterns herein described or depicted specifically. Rather, those of ordinary skill in the art will appreciate that the phrase is included to provide for obvious modifications, which are encompassed within the scope of the appended claims.

The present invention includes one or more of the compounds of Formula I for use in the treatment of conditions or disorders affected by selective estrogen receptor modulation in a mammal (such as a human) in need thereof. In one embodiment, the present invention provides methods for the treatment of conditions or disorders selected from List A:

List A (conditions or disorders affected by selective estrogen receptor modulation and treatable by the compounds of Formula I): osteoporosis, bone demineralization, reduced bone mass, density, or growth, osteoarthritis, acceleration of bone fracture repair and healing, acceleration of healing in joint replacement, periodontal disease, acceleration of tooth repair or growth, Paget's disease, osteochondrodysplasias, muscle wasting, the maintenance and enhancement of muscle strength and function, frailty or age-related functional decline (“ARFD”), sarcopenia, chronic fatigue syndrome, chronic myaligia, acute fatigue syndrome, acceleration of wound healing, maintenance of sensory function, chronic liver disease, AIDS, weightlessness, burn and trauma recovery, thrombocytopenia, short bowel syndrome, irritable bowel syndrome, inflammatory bowel disease, Crohn's disease and ulcerative colitis, obesity, eating disorders including anorexia associated with cachexia or aging, hypercortisolism and Cushing's syndrome, cardiovascular disease or cardiac dysfunction, congestive heart failure, high blood pressure, breast cancer, malignant tumor cells containing the androgen receptor including breast, brain, skin, ovary, bladder, lymphatic, liver, kidney, uterine, pancreas, endometrium, lung, colon, and prostate, prostatic hyperplasia, hirsutism, acne, seborrhea, androgenic alopecia, anemia, hyperpilosity, adenomas and neoplasis of the prostate, hyperinsulinemia, insulin resistance, diabetes, syndrome X, dyslipidemia, urinary incontinence, artherosclerosis, libido enhancement, sexual dysfunction, depression, depressive symptoms, nervousness, irritability, stress, reduced mental energy and low self-esteem, improvement of cognitive function, endometriosis, polycystic ovary syndrome, counteracting preeclampsia, premenstral syndrome, contraception, uterine fibroid disease, and/or aortic smooth muscle cell proliferation, vaginal dryness, pruritis, dyspareunia, dysuria, frequent urination, urinary tract infections, hypercholesterolemia, hyperlipidemia, peripheral vascular disease, restenosis, vasospasm, vascular wall damage due to immune responses, Alzheimer's disease, bone disease, aging, inflammation, rheumatoid arthritis, respiratory disease, emphysema, reperfusion injury, viral hepatitis, tuberculosis, psoriasis, systemic lupus erythematosus, amyotrophic lateral sclerosis, stroke, CNS trauma, dementia, neurodegeneration, breast pain and dysmenorrhea, menopausal or postmenopausal disorders, vasomotor symptoms, urogenital or vulvar vaginal atrophy, atrophic vaginitis, female sexual dysfunction, for enhancing libido, for the treatment of hypoactive sexual disorder, sexual arousal disorder, for increasing the frequency and intensity of orgasms, vaginismus, osteopenia, endometriosis, BPH (benign prostatic hypertrophy), dysmenorrhea, autoimmune diseases, Hashimoto's thyroiditis, SLE (systemic lupus erythematosus), myasthenia gravis, or reperfusion damage of ischemic myocardium. More preferably the treatment relates to menopausal or postmenopausal disorders, -vasomotor symptoms, urogenital or vulvar vaginal atrophy, atrophic vaginitis, endometriosis, female sexual dysfunction, breast cancer, depressive symptoms, diabetes, bone demineralization, or osteoporosis.

Also, the present invention includes the use of one or more of the compounds of Formula I in the manufacture of a medicament for use in the treatment of conditions or disorders associated with selective estrogen receptor modulation. Preferably the medicament is for use in the treatment of those conditions and disorders of List A, above.

The present invention includes a method for the treatment of conditions or disorders associated with selective estrogen receptor modulation comprising the administration of at least one compound of Formula I. Preferably the treatment relates to the conditions and disorders of List A, above.

The compounds of formula I, or salts or solvates thereof, may be advantageous in the treatment of menopausal or postmenopausal disorders.

The compounds of formula I, or salts or solvates thereof, may be advantageous in the treatment of vasomotor symptoms.

The compounds of formula I, or salts or solvates thereof, may be advantageous in the treatment of urogenital or vulvar vaginal atrophy.

The compounds of formula I, or salts or solvates thereof, may be advantageous in the treatment of atrophic vaginitis.

The compounds of formula I, or salts or solvates thereof, may be advantageous in the treatment of endometriosis.

The compounds of formula I, or salts or solvates thereof, may be advantageous in the treatment of female sexual dysfunction.

The compounds of formula I, or salts or solvates thereof, may be advantageous in the treatment of breast cancer.

The compounds of formula I, or salts or solvates thereof, may be advantageous in the treatment of depressive symptoms.

The compounds of formula I, or salts or solvates thereof, may be advantageous in the treatment of diabetes.

The compounds of formula I, or salts or solvates thereof, may be advantageous in the treatment of bone demineralization.

The compounds of formula I, or salts or solvates thereof, may be advantageous in the treatment of osteoporosis.

In particular, the compounds of the present invention are believed useful, either alone or in combination with other agents, in the treatment of menopausal or postmenopausal disorders, vasomotor symptoms, urogenital or vulvar vaginal atrophy, atrophic vaginitis, female sexual dysfunction, breast cancer, depressive symptoms, diabetes, bone demineralization, and the treatment of osteoporosis.

As used herein, the term “effective amount” means that amount of a drug or pharmaceutical agent that will elicit the biological or medical response of a tissue, system, animal or human that is being sought, for instance, by a researcher or clinician. The term “therapeutically effective amount” means any amount which, as compared to a corresponding subject who has not received such amount, results in treatment, healing, prevention, or amelioration of a disease, disorder, or side effect, or a decrease in the rate of advancement of a disease or disorder. The term also includes within its scope amounts effective to enhance normal physiological function.

A therapeutically effective amount of a compound of the present invention will depend upon a number of factors. For example, the age and weight of the animal, the precise condition requiring treatment and its severity, the nature of the formulation, and the route of administration are all factors to be considered. The therapeutically effective amount ultimately should be at the discretion of the attendant physician or veterinarian. For example, an effective amount of a compound of formula I for the treatment of humans suffering from osteoporosis, generally, should be in the range of 0.1 to 100 mg/kg body weight of recipient (mammal) per day. More usually the effective amount should be in the range of 1 to 10 mg/kg body weight per day. Thus, for a 70 kg adult mammal the actual amount per day would usually be from 70 to 700 mg. This amount may be given in a single dose per day or in a number (such as two, three, four, five, or more) of sub-doses per day such that the total daily dose is the same. An effective amount of a salt or solvate thereof, may be determined as a proportion of the effective amount of the compound of formula I per se. Similar dosages should be appropriate for treatment of the other conditions referred to herein that are mediated by estrogen.

For use in therapy, therapeutically effective amounts of a compound of formula I, as well as salts and solvates thereof, may be administered as the raw chemical. Additionally, the active ingredient may be presented as a pharmaceutical composition. Accordingly, the invention further provides pharmaceutical compositions that include effective amounts of compounds of the Formula I and salts and solvates thereof, and one or more pharmaceutically acceptable carriers, diluents, or excipients. The compounds of Formula I and salts or solvates thereof, are as described above. The carrier(s), diluent(s) or excipient(s) must be acceptable, in the sense of being compatible with the other ingredients of the formulation and not deleterious to the recipient of the pharmaceutical composition.

In accordance with another aspect of the invention there is also provided a process for the preparation of a pharmaceutical formulation including admixing a compound of the Formula I or salts and solvates thereof, with one or more pharmaceutically acceptable carriers, diluents or excipients.

Pharmaceutical formulations may be presented in unit dose forms containing a predetermined amount of active ingredient per unit dose. Such a unit may contain, as a non-limiting example, 0.5 mg to 1 g of a compound of the formula I, depending on the condition being treated, the route of administration, and the age, weight, and condition of the patient. Preferred unit dosage formulations are those containing a daily dose or sub-dose, as herein above recited, or an appropriate fraction thereof, of an active ingredient. Such pharmaceutical formulations may be prepared by any of the methods well known in the pharmacy art.

Pharmaceutical formulations may be adapted for administration by any appropriate route, for example by an oral (including buccal or sublingual), rectal, nasal, topical (including buccal, sublingual or transdermal), vaginal, or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions, each with aqueous or non-aqueous liquids; edible foams or whips; or oil-in-water liquid emulsions or water-in-oil liquid emulsions. For instance, for oral administration in the form of a tablet or capsule, the active drug component can be combined with an oral, non-toxic pharmaceutically acceptable inert carrier such as ethanol, glycerol, water, and the like. Generally, powders are prepared by comminuting the compound to a suitable fine size and mixing with an appropriate pharmaceutical carrier such as an edible carbohydrate, as, for example, starch or mannitol. Flavorings, preservatives, dispersing agents, and coloring agents can also be present.

Capsules are made by preparing a powder, liquid, or suspension mixture and encapsulating with gelatin or some other appropriate shell material. Glidants and lubricants such as colloidal silica, talc, magnesium stearate, calcium stearate or solid polyethylene glycol can be added to the mixture before the encapsulation. A disintegrating or solubilizing agent such as agar-agar, calcium carbonate or sodium carbonate can also be added to improve the availability of the medicament when the capsule is ingested. Moreover, when desired or necessary, suitable binders, lubricants, disintegrating agents, and coloring agents can also be incorporated into the mixture. Examples of suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes, and the like. Lubricants useful in these dosage forms include, for example, sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like. Tablets are formulated, for example, by preparing a powder mixture, granulating or slugging, adding a lubricant and disintegrant, and pressing into tablets. A powder mixture may be prepared by mixing the compound, suitably comminuted, with a diluent or base as described above. Optional ingredients include binders, such as carboxymethylcellulose, aliginates, gelatins, or polyvinyl pyrrolidone, solution retardants, such as paraffin, resorption accelerators such as a quaternary salt and/or absorption agents such as bentonite, kaolin, or dicalcium phosphate. The powder mixture can be wet-granulated with a binder such as syrup, starch paste, acadia mucilage or solutions of cellulosic or polymeric materials, and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material, and a polish coating of wax can be provided. Dyestuffs can be added to these coatings to distinguish different unit dosages.

Oral fluids such as solutions, syrups, and elixirs can be prepared in dosage unit form so that a given quantity contains a predetermined amount of the compound. Syrups can be prepared, for example, by dissolving the compound in a suitably flavored aqueous solution, while elixirs are prepared through the use of a non-toxic alcoholic vehicle. Suspensions can be formulated generally by dispersing the compound in a non-toxic vehicle. Solubilizers and emulsifiers such as ethoxylated isostearyl alcohols and polyoxy ethylene sorbitol ethers, preservatives; flavor additives such as peppermint oil, or natural sweeteners, saccharin, or other artificial sweeteners; and the like can also be added.

Where appropriate, dosage unit formulations for oral administration can be microencapsulated. The formulation can also be prepared to prolong or sustain the release as for example by coating or embedding particulate material in polymers, wax or the like.

The compounds of Formula I and salts and solvates thereof, can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

The compounds of Formula I and salts or solvates thereof may also be delivered by the use of monoclonal antibodies as individual carriers to which the compound molecules are coupled. The compounds may also be coupled with soluble polymers as targetable drug carriers. Such polymers can include polyvinylpyrrolidone (PVP), pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug; for example, polylactic acid, polyepsilon caprolactone, polyhydroxy butyric acid, polyorthoesters, polyacetals, polydihydropyrans, polycyanoacrylates, and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986), incorporated herein by reference as related to such delivery systems.

Pharmaceutical formulations adapted for topical administration may be formulated as ointments, creams, suspensions, lotions, powders, solutions, pastes, gels, sprays, aerosols, or oils.

For treatments of the eye or other external tissues, for example mouth and skin, the formulations may be applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical formulations adapted for topical administrations to the eye include eye drops wherein the active ingredient is dissolved or suspended in a suitable carrier, especially an aqueous solvent. Pharmaceutical formulations adapted for topical administration in the mouth include lozenges, pastilles, and mouthwashes.

Pharmaceutical formulations adapted for nasal administration, where the carrier is a solid, include a coarse powder having a particle size for example in the range 20 to 500 microns. The powder is administered in the manner in which snuff is taken, i.e., by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurized aerosols, nebulizers, or insufflators.

Pharmaceutical formulations adapted for rectal administration may be presented as suppositories or as enemas.

Pharmaceutical formulations adapted for vaginal administration may be presented as pessaries, tampons, creams, gets, pastes, foams, or spray formulations.

Pharmaceutical formulations adapted for parenteral administration include aqueous and non-aqueous sterile injection solutions which may contain anti-oxidants, buffers, bacteriostats, and solutes that render the formulation isotonic with the blood of the intended recipient; and aqueous and non-aqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

In addition to the ingredients particularly mentioned above, the formulations may include other agents conventional in the art having regard to the type of formulation in question. For example, formulations suitable for oral administration may include flavoring agents.

The compounds of the present invention and their salts or solvates thereof, may be employed alone or in combination with other therapeutic agents for the treatment of the conditions described in List A above. For example, in osteoporosis therapy, combination with other osteoporosis therapeutic agents is envisaged. Osteoporosis combination therapies according to the present invention thus comprise the administration of at least one compound of formula I or a salt or solvate thereof, and the use of at least one other osteoporosis treatment method. Preferably, combination therapies according to the present invention comprise the administration of at least one compound of Formula I or a salt or solvate thereof, and at least one other osteoporosis treatment agent, for example, a bone building agent. As a further example, combination therapies according to the present invention include the administration of at least one compound of the present invention or a salt or solvate thereof, and at least one other osteoporosis treatment agent, for example, an anti-bone resorption agent. As noted, one potential additional osteoporosis treatment agent is a bone building (anabolic) agent. Bone building agents can lead to increases in parameters such as bone mineral density that are greater than those than can be achieved with anti-resorptive agents. In some cases, such anabolic agents can increase trabecular connectivity leading to greater structural integrity of the bone.

The compound(s) of Formula I and the other pharmaceutically active agent(s) may be administered together or separately and, when administered separately, administration may occur simultaneously or sequentially in any order. The amounts of the compound(s) of Formula I and the other pharmaceutically active agent(s) and the relative timings of administration will be selected in order to achieve the desired combined therapeutic effect. The administration in combination of a compound of Formula I salts or solvates thereof with other osteoporosis treatment agents may be in combination by administration concomitantly in: (1) a unitary pharmaceutical composition including each compound; or (2) separate pharmaceutical compositions each including one of the compounds. Alternatively, the combination may be administered separately in a sequential manner wherein one treatment agent is administered first and the other(s) subsequently or vice versa. Such sequential administration may be close in time or remote in time.

Other potential therapeutic combinations include the compounds of the present invention combined with other compounds of the present invention, growth promoting agents, growth hormone secretagogues, growth hormone releasing factor and its analogs, growth hormone and its analogs, somatomedins, alpha-ardenergic agonists, serotonin 5-HT_{D} agonists, selective serotonin reuptake inhibitors, agents that inhibit somatostatin or its release, 5-α-reductase inhibitors, aromatase inhibitors, GnRH inhibitors, parathyroid hormone, bisphosphonates, estrogen, testosterone, SERMs, progesterone receptor agonists, and/or with other modulators of nuclear hormone receptors.

In the context of treatment for the various diseases mentioned above, the compounds of the present invention may also be combined with additional therapeutic agents selected for the treatment of other symptoms or conditions which may accompany or exist together with the conditions or diseases, the treatment of which is the subject of the present invention. For example, the compounds of the present invention may be used in combination with anti-diabetic agents, anti-osteoporosis agents, anti-obesity agents, anti-inflammatory agents, anti-anxiety agents, anti-depressants, anti-hypertensive agents, anti-platelet agents, anti-thrombotic and thrombolytic agents, cardiac glycosides, cholesterol or lipid lowering agents, mineralocorticoid receptor antagonists, phosphodiesterase inhibitors, kinase inhibitors, thyroid mimetics, anabolic agents, viral therapies, cognitive disorder therapies, sleeping disorder therapies, sexual dysfunction therapies, contraceptives, cytotoxic agents, radiation therapy, anti-proliferative agents, and anti-tumor agents. Additionally, the compounds of the present invention may be combined with nutritional supplements such as amino acids, triglycerides, vitamins, minerals, creatine, piloic acid, carnitine, or coenzyme Q10.

The compounds of this invention may be made by a variety of processes, including well-known standard synthetic methods. Illustrative general synthetic methods are set out below and then specific compounds of the invention are prepared in the working Examples.

In all of the examples described below, protecting groups for sensitive or reactive groups are employed where necessary in accordance with general principles of synthetic chemistry. Protecting groups are manipulated according to standard methods of organic synthesis (T. W. Green and P. G. M. Wuts (1991) Protecting Groups in Organic Synthesis, John Wiley & Sons, incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of Formula I.

Those skilled in the art will recognize if a chiral center exists in compounds of Formula I Accordingly, the present invention includes all possible stereoisomers and includes not only racemic compounds but the individual enantiomers as well. When a compound is desired as a single enantiomer, such may be obtained by stereospecific synthesis, by resolution of the final product or any convenient intermediate, or by chiral chromatographic methods as are known in the art. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994), incorporated by reference with regard to stereochemistry.

Since the compounds of formula (I) are intended for use in pharmaceutical compositions it will readily be understood that they are each advantageously provided in substantially pure form, for example at least 60% pure, more suitably at least 75% pure and advantageously at least 85%, especially at least 98% pure (% are on a weight for weight basis). Impure preparations of the compounds may be used for preparing the more pure forms used in the pharmaceutical compositions.

### Experimental Section

### Abbreviations:

As used herein the symbols and conventions used in these processes, schemes and examples are consistent with those used in the contemporary scientific literature, for example, the Journal of the American Chemical Society or the Journal of Biological Chemistry. Specifically, the following abbreviations may be used in the examples and throughout the specification:

| | |
|---|---|
| g (grams); | mg (milligrams); |
| L (liters); | mL (milliliters); |
| μL (microliters); | psi (pounds per square inch); |
| M (molar); | mM (millimolar); |
| Hz (Hertz); | MHz (megahertz); |
| mol (moles); | mmol (millimoles); |
| RT (room temperature); | h (hours); |
| d (days); | EI (electron impact); |
| min (minutes); | TLC (thin layer chromatography); |
| mp (melting point); | RP (reverse phase); |
| Tᵣ (retention time); | TFA (trifluoroacetic acid); |
| TEA (triethylamine); | THF (tetrahydrofuran); |
| TFAA (trifluoroacetic anhydride); | CD₃OD (deuterated methanol); |
| CDCl₃ (deuterated chloroform); | DMSO (dimethylsulfoxide); |
| SiO₂ (silica); | atm (atmosphere); |
| EtOAc (EtOAc); | CHCl₃ (chloroform); |
| HCl (hydrochloric acid); | Ac (acetyl); |
| DMF (N,N-dimethylformamide); | Me (methyl); |
| Cs₂CO₃ (cesium carbonate); | EtOH (ethanol); |
| Et (ethyl); | tBu (tert-butyl); |
| MeOH (methanol); | CH₂Cl₂ (dichloromethane); |
| MgSO₄ (magnesium sulfate); | CH₃CN (acetonitrile); |
| K₂CO₃ (potassium carbonate); | TiCl₄ (titanium tetrachloride); |
| EtOAc (EtOAc); | CO₂ (carbon dioxide); |
| Pd(OAc)₂ (palladium acetate); | Et₂O (diethyl ether); |
| P(o-tolyl)₃ (tri-o-tolylphosphine); | Na₂SO₄ (sodium sulfate); |
| NaH (sodium hydride); | DME (1,2-dimethoxyethane); |
| NaI (sodium iodide); | NaOH (sodium hydroxide); |
| NH₄Cl (ammonium chloride); | NaHCO₃ (sodium bicarbonate); |
| AlCl₃ (aluminum chloride); | (C₂H₅O)₂P(O)H (diethyl phosphite); |
| NaN₃ (sodium azide); | CBr₄ (carbon tetrabromide); |
| PPh (triphenylphosphine); | CuI (copper (I) iodide); |
| Pd(Ph₃P)₄ (tetrakis(triphenylphosphine)palladium (0)); | nBuLi (butyllithium); |
| (iPrO)₃B (triisopropyl borate); | DMAP (4-(dimethylamino)pyridine); |
| Na₂CO₃ (sodium carbonate); | |
| eq (equivalents); | |
| HRMS (high resolution mass spectrometry); | |
| LCMS (liquid chromatography mass spectrometry); | |
| LRMS (low resolution mass spectrometry); | |
| APCI (Atmospheric Pressure Chemical Ionization); | |
| LiHMDS (lithium bis(trimethylsilyl)amide); | |
| Pd(Ph₃P)₂Cl₂ (dichlorobis(triphenylphosphine)palladium(II)); | |
| EDC (N-(3-dimethylaminopropyl)-N′-ethyl-carbodimide; | |
| dpppe (1,5-bis(diphenylphosphanyl)pentane; | |
| DMAc (N,N-dimethylacetamide); | |
| HPLC (high performance liquid chromatography); | |
| tmeda (N,N,N′,N′,-tetramethylethylenediamine); | |
| Pd₂(dba)₃ (dipalladiumtris(dibenzylidene acetone)); | |
| OTBDPS (O-ter-butyldimethylsilane); | |
| OMOM (O-methoxy-O-methyl); and | |
| OBz (O-benzyl). | |

Unless otherwise noted, reagents and solvents were obtained from commercial suppliers and were used without further purification. Unless otherwise indicated, all reactions were conducted at room temperature and all temperatures are expressed in ° C. (degrees Centigrade).

Thin-layer chromatography (TLC) was performed on silica gel 60 F₂₅₄ precoated plates. Detection was effected by exposure to UV light (254 nm). Flash and flush column chromatography was performed using Silica Gel 60. Reverse phase preparative and analytical HPLC were performed using C18 columns and acetonitrile:water gradients with 0.05% TFA as a modifier.

Compound purity and characterization were determined by ¹H-NMR, liquid chromatography-mass spectrometry (LCMS), high resolution mass spectrometry (HRMS), combustion (elemental) analysis, HPLC, and melting point. Compounds of general formula I were typically found to have purities of >90%.

¹H NMR spectra were recorded on Varian INOVA-300 and Varian INOVA-400 instruments. Chemical shifts are expressed in parts per million (ppm, δ units). Coupling constants are in units of hertz (Hz). Splitting patterns describe apparent multiplicities and are designated as s (singlet), d (doublet), dd (doublet of doublet), t (triplet), q (quartet), m (multiplet), or br (broad).

Low resolution mass spectra were obtained on Micromass ZQ, Micromass ZMD, Micromass QuattroMicro, and Micromass GCT instruments from Micromass Ltd., Altricham, UK, using either Atmospheric Pressure Chemical Ionization (APCI) or ESI Ionization (ESI).

High resolution mass spectral data (HRMS) were recorded with Micromass LCT and Micromass GCT instruments.

Combustion analyses were performed by Atlantic Microlab, Inc. (Norcross, Ga.).

Melting points were recorded in open capillary tubes and are uncorrected.

The bolded numerals reference the compounds as depicted in the following schemes. For the following schemes, depending on subsequent chemistry and functional group compatibility, the phenol groups of specific intermediates may need to be protected using synthetic methods appreciated by those skilled in the art.

### SYNTHETIC SCHEMES

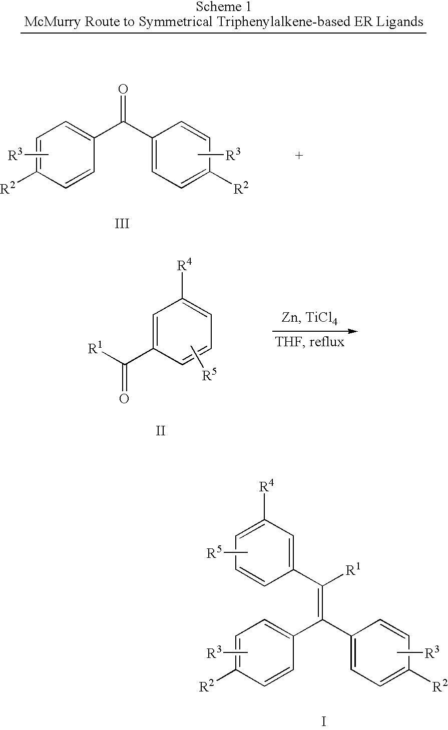

The symmetrical triphenylalkene compound I can be prepared following the route described in Scheme 1. McMurry coupling between substituted benzophenone III and substituted phenyl alkyl ketone II provides the triphenylalkene 1. For McMurry reaction conditions, see Mukaiyama et al., Chem. Lett. (1973), 1041; Lenoir, Synthesis, (1977), 553; Lenoir and Burghard, J. Chem. Res. (S) (1980), 396; McMurry, Chem. Rev. (1989), 89, 1513-1524; McMurry, Acc. Chem. Res. (1983) 16, 405-511; and S. Gauthier et al., J. Org. Chem., (1996), 61, 3890-3893, each herein incorporated by reference with regard to such teaching. Ketones II and III are either commercially available or may be prepared by synthetic methods (Scheme 2, for example) appreciated by those skilled in the art of organic chemistry.

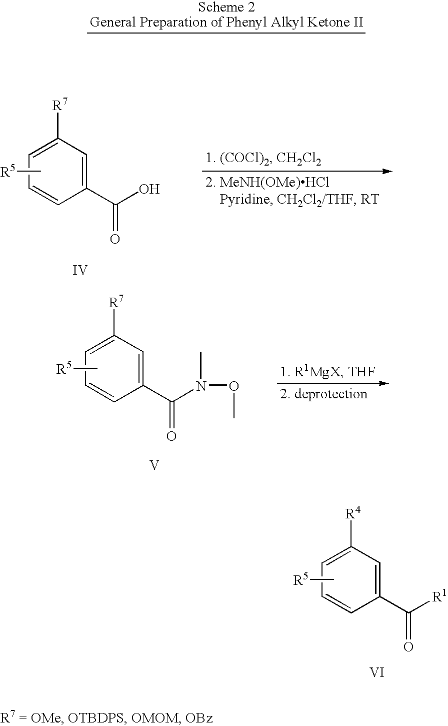

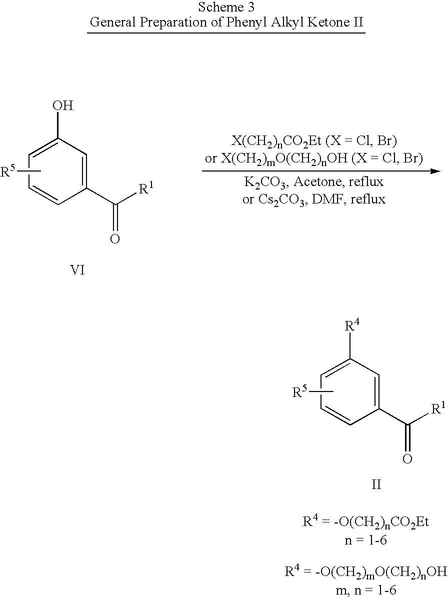

Reaction of acid chloride derived from the acid IV with N,O-dimethyl hydroxylamine hydrochloride yields the Weinreb amide V. The acid chloride can be prepared from the acid IV using the standard procedure described in the art. Treatment of amide V with a Grignard reagent followed by demethylation affords compound VI. For general reaction conditions, see S, Nahm and S. M. Weinreb Tetrahedron Lett. (1981), 22, 3815. B. M. Kim, et al, Tetrahedron Lett. (1994), 35, 5153, for review, see M. P. Sibi, Org. prep. Proc. Intl. (1993), 25, 15, each herein incorporated by reference with regard to such teaching. The phenyl alkyl ketone II can be prepared from VI using the standard alkylation procedure described in the art of organic chemistry. Similarly, synthesis of VI in which the hydroxyl group is ortho to the carbonyl can be achieved using an identical procedure.

Triphenylalkene I can also be prepared using the procedure described in Scheme 4. 1,1-dibromo-1-alkene VII can be prepared from alkyl phenyl ketone II using the procedure reported by Corey and Fuchs (see E. J. Corey and P. L. Fuchs, Tetrahedron Lett. (1972), 3769. Alternatively the dibromocompound VII can also be prepared using the procedure reported by V. G. Nenajdenko, et al J. Chem. Soc., Perkin Trans. I, (2002), 883, J. F. Normant et al Synthesis (2000), 109. Dibromo alkene VII can be coupled with a variety of aryl boronic acids VIII using Suzuki reaction conditions to afford triphenylalkene 1. For general Suzuki coupling reaction conditions, see, Miyaura, N., Suzuki, A. Chem. Rev. (1995), 95, 2457-2483; Suzuki, A., J. Organometallic Chem. (1999), 576, 147-168; and Suzuki, A. in Metal-catalyzed Cross-coupling Reactions, Diederich, F., and Stang, P. J., Eds.; Wiley-VCH: New York, (1998), pp. 49-97, each herein incorporated by reference with regard to such teaching. For Suzuki coupling reaction conditions of 1,1-dibromo-1-alkene, see M. W. Miller et al., Synlett (2001), 254, herein incorporated by reference with regard to such teaching. The dibromo alkene VII can also be transformed to 1,1-diboryl-1-alkene intermediate, which upon reaction with aryl halides can generate 1,1,2-triaryl-alkenes III. For related transformations, see M. Shimizu et al., J. Am. Chem. Soc., (2005), 127, 12506, herein incorporated by reference with regard to such teaching.

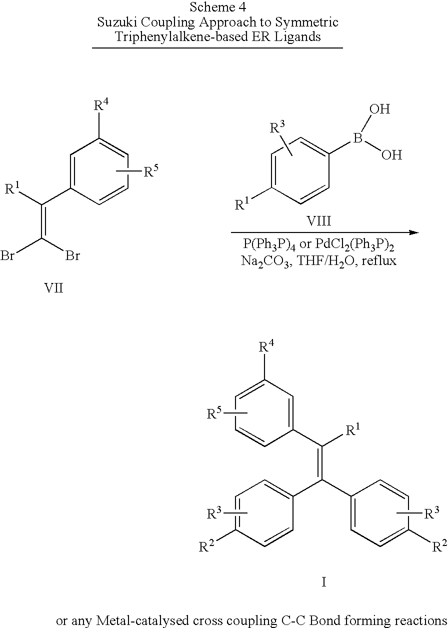

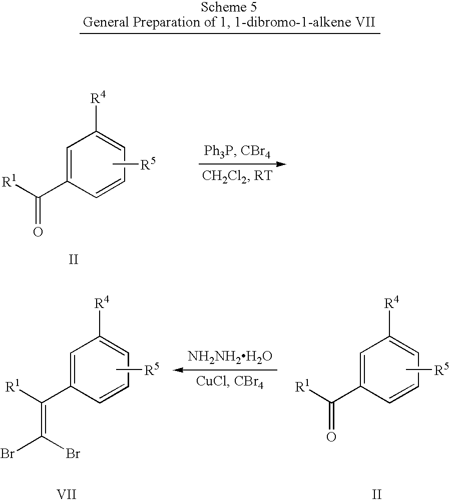

Further functionalization of the R⁴ substituent of intermediate IX (Scheme 6) can be carried out. For example, when R⁴ is Br or I, Heck coupling yields an acrylic ester, which upon hydrolysis affords acrylic acid X. Acrylic acid in turn can be converted to the corresponding carboxamide XI. For reviews of the Heck coupling reaction, see Heck, Acc. Chem. Res. (1979), 12, 146-151; Heck, Pure Appl. Chem. (1978), 50, 691-701; R. F. Heck, Palladium Reagents in Organic Syntheses, Academic Press, New York (1985), 179-321, Bender, Stakem, and Heck, J. Org. Chem. (1982), 47, 1278; Spencer, J. Organomet. Chem. (1983), 258, 101; and Brase, Stefan; De Meijere, Armin. Palladium-catalyzed Coupling of aryl halides to Alkenes—the Heck Reaction, Metal-Catalyzed Cross-Coupling Reactions (1998), 99-166, Publisher: Wiley-VCH Verlag GmbH, Weinheim, Germany, each herein incorporated by reference with regard to such teaching.

Suzuki coupling of 1× with a variety of boronic acids provides biaryl compound XII. For conditions of the Suzuki coupling reaction, see, Miyaura, N., Suzuki, A. Chem. Rev. 1995, 95, 2457-2483; Suzuki, A., J. Organometallic Chem. (1999), 576, 147-168; and Suzuki, A. in Metal-catalyzed Cross-coupling Reactions, Diederich, F., and Stang, P. J., Eds.; Wiley-VCH: New York, (1998), pp. 49-97, each herein incorporated by reference with regard to such teaching.

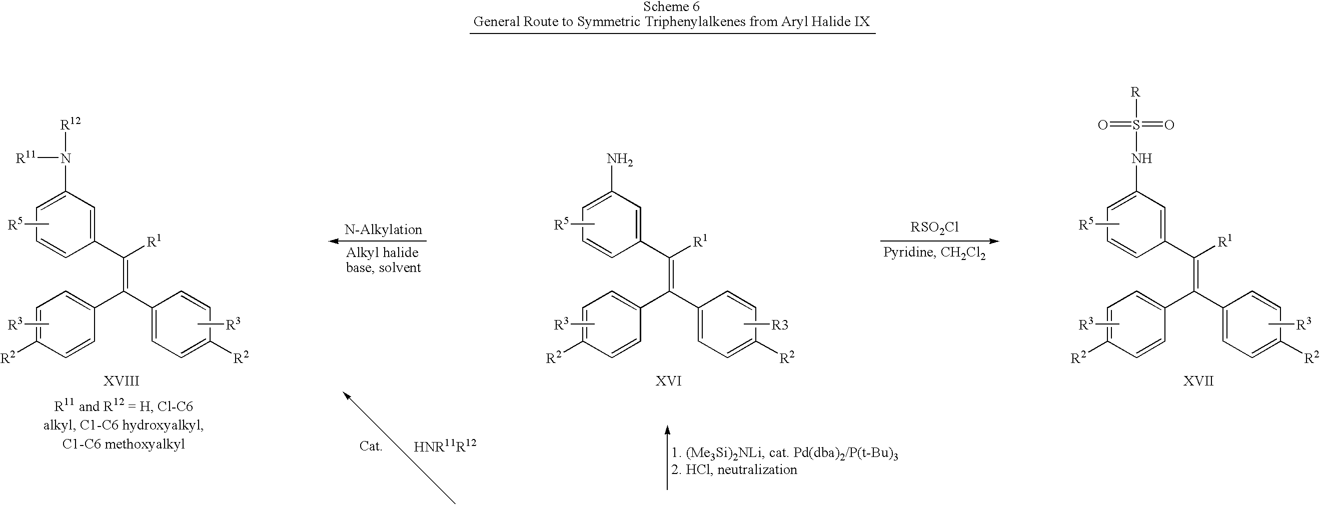

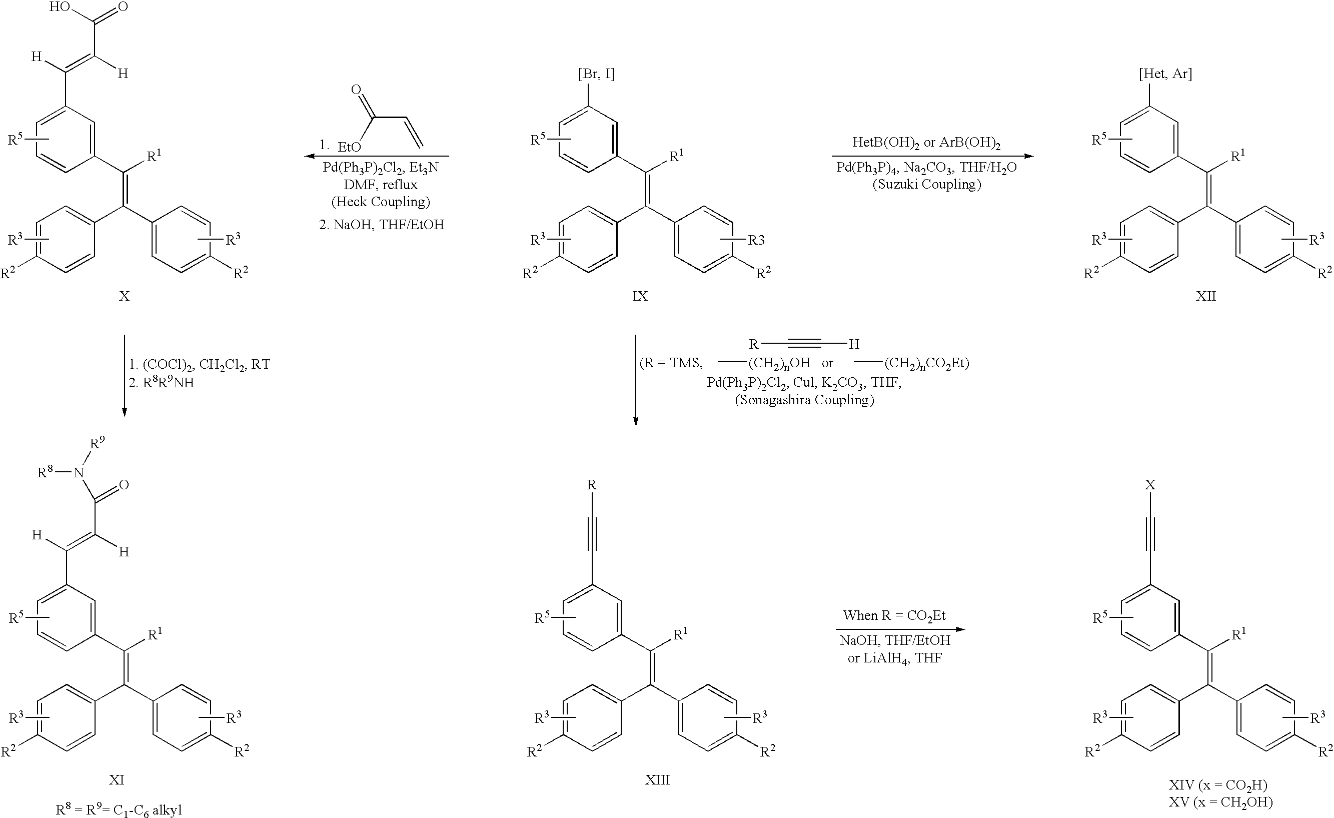

Sonogashira coupling of 1× with a propiolate ester, propiolate alcohol or (trimethylsilyl)acetylene gives aromatic alkyne XIII. For details of Sonogashira reaction conditions, see Campbell, I. B. “The Sonogashira Cu—Pd-catalyzed alkyne coupling reaction” in Organocopper Reagents, Taylor, Richard J. K. ed., (1994), 217-35. Publisher: IRL Press, Oxford, UK; G. C. Nwokogu et al., J. Org. Chem., (1994), 59 (9), 2506-2510; and A. P. Kozikowski J. Med. Chem. (2000), 43 (6), 1215-1222 and T. Eckert and J. Ipaktschi Synth. Commun. (1998), 28, 327-336, each herein incorporated by reference with regard to such teaching. Compound XIII can be further elaborated to provide additional new analogues. For example when R═CO₂Et, saponification gives the acid XIV and reduction with LAH provides the corresponding primary alcohol XV.

Compound IX can be converted to primary amine XVI using lithium bis (trimethylsilyl) amide and a palladium catalyst. For a description of the conversion of aromatic halides to the corresponding anilines, see Hartwig, J. H. et al. Org. Let. (2001), 2729, herein incorporated by reference with regard to such teaching. Amine XVI can be further converted to sulfonamide XVII using reaction conditions familiar to those skilled in the art of organic chemistry. For a representative procedure, see L is, R. et al. J. Org. Chem. (1987), 52, 4377, herein incorporated by reference with regard to such teaching. Amine XVI can also be transformed to the N-alkylated product XVIII using well-established procedures familiar to those skilled in the art. Compound XVIII can also be prepared directly from 1× using palladium catalyzed cross-coupling reactions. For reaction conditions, see S. L. Buchwald et al., Acc. Chem. Res. (1998), 31, 805, J. F. Hartwig, Angew. Chem. Int. Ed. (1998), 37, 2046. each herein incorporated by reference with regard to such teaching.

An O-alkylation of XIX (Scheme 7) with alkyl halides provides compounds XX and XXI. Compound XX and XXI can be further subjected to prepare additional analogues. For example, when R=Et, hydrolysis of XX gives XXI and hydrolysis of XXII gives XXXIII, the corresponding alkanoic acids. Reduction of XX and XXII with LAH provides alkanoic alcohols XXIV and XXV respectively. Compound XXV can also be obtained via alkylation of XIX with a suitable alkylating agent. For examples of related phenol alkylation reactions see Rubin, V. et al., Bioorganic & Med. Chem. (2001), 9, 1579-1586, S. Gauthier et al, J. org. Chem. (1996), 61, 3890, each herein incorporated by reference with regard to such teaching.

Similarly, the alkylation of XIX with haloacetonitrile gives alkylated product XXVII, which upon LAH reduction provides the corresponding primary amine XXVIII. The resulting amine, following alkylation using well established reaction conditions, provides N-alkylated product XXIX. Compound XIX can be converted to XXX using Mitsunobu conditions. For general reaction conditions, O. Mitsunobu, Synthesis (1981), 1-28, D. L. Hughes, Organic Preparations and Procedures Int. (1996), 28, 127-164, each herein incorporated by reference with regard to such teaching.

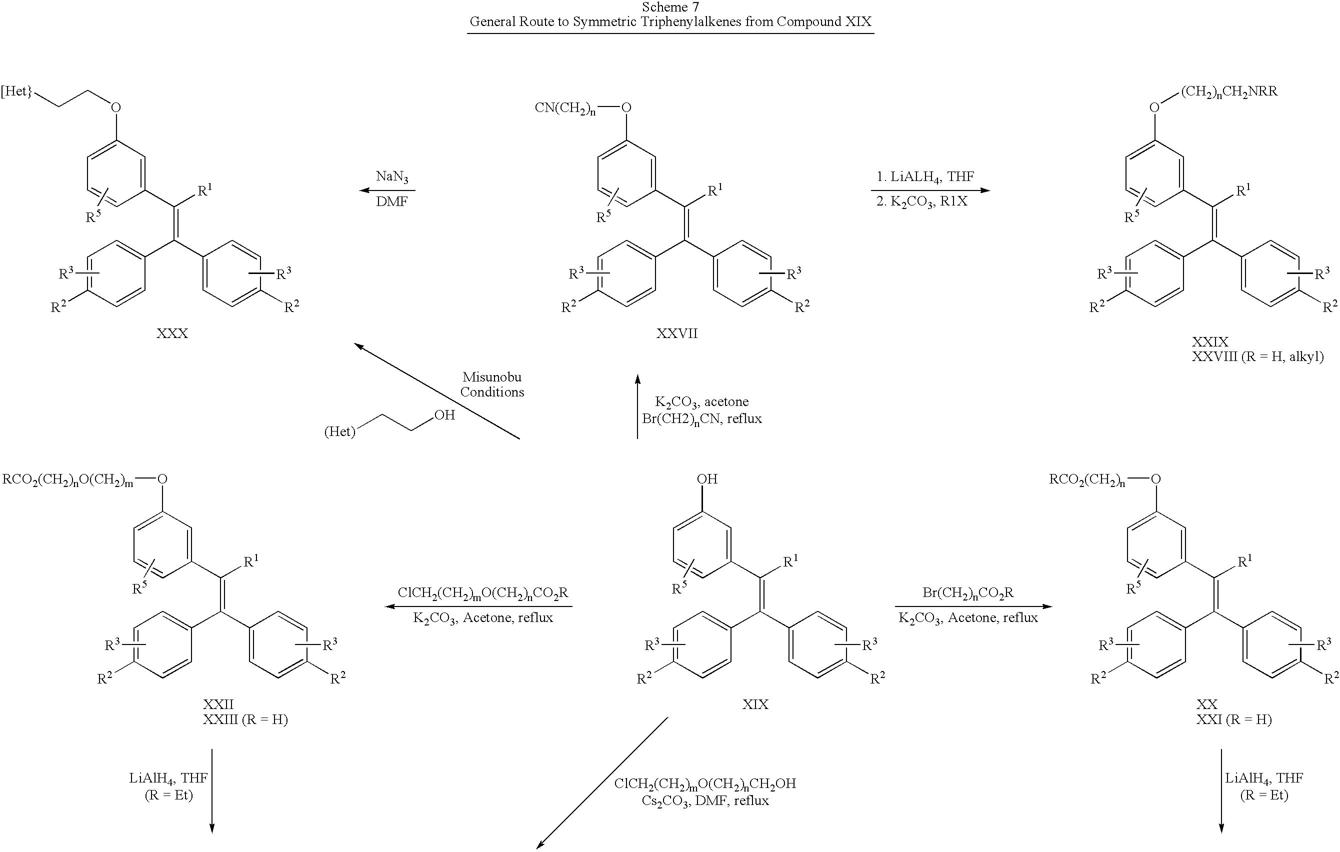

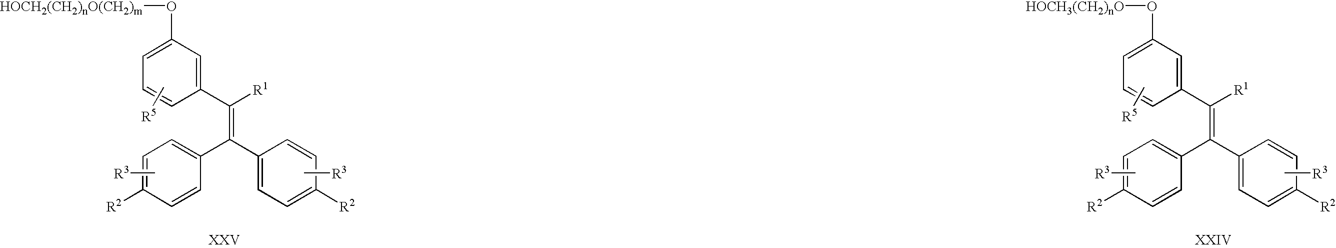

Alkanoic acids can be further converted to their corresponding carboxamides. Acids can be converted to the corresponding acid chlorides and then treated with amines to afford the corresponding carboxamides. The conversion of carboxylic acids to carboxamides is well known in the art of organic synthesis. For the details, see Larock, R. C., Comprehensive Organic Transformations, VCH Publishers, New York, 1989.

Variety of compounds can be prepared from compound IX (Scheme 8). For example metal-halogen exchange with n-butyllithium and DMF provides aldehyde XXXI. For reaction conditions, see T. Mizuno et al., Tetrahedron, (1999), 55(31), 9455-9468; J. W. Lampe et al., J. Med. Chem., (2002), 45(12), 2624-2643; R. G. Leenders et al., Bioorg. Med. Chem. (1999), 7(8), 1597-1610; and A. Endo et al., J. Amer. Chem. Soc., (2002), 124(23), 6552-6554, each herein incorporated by reference. The aldehyde XXXI can be converted to the oxime XXXII. The halide IX can also be converted alkyl or aryl sulfones using the procedure described in the art. For reaction conditions, see Zhu, W, and Ma, D, J. Org. Chem. 2005, 70, 2696, herein incorporated such teaching. The aldehyde XXXI can also be converted to nitrite XXXIV. For the details of reaction conditions, see R. Ballini et al., Synlett. (2003), 1841, herein incorporated by reference with regard to such teaching. The nitrite XXXIV can also be obtained using CuCN/DMF conditions. The nitrile XXXIV can serve as a good precursor for the preparation of a heterocyclic compounds. For reaction conditions, see M. Chichiro et al J. Med. Chem. (1995), 38, 353, I. K. Kanna, et al. J. Med. Chem. (1997), 40, 1634, B. D. Judkins, et al, Synth. Commun. (1996), 26, 4351, each herein incorporated by reference. The aldehyde XXXI can also further be converted to new derivatives. For example Wittig reaction/Horner-Wadsworth-Emmons reaction, using the standard conditions described in the art, followed by hydrolysis of the ester gives X. Alternatively, compound X can also be obtained from XXXI by Knoevenagel condensation/Doebner condensation with malonic acid, pyridine, and a catalytic amount of morpholine. For reaction conditions, see E. Knoevenagel, Ber. (1898), 31, 2596, G. Jones, Org. Reactions (1967), 15, 204, R. W. Draper et al Tetrahedron (2000), 56, 1811, each herein incorporated by reference with regard to such teaching. The versatile intermediate IX can also be used for a variety of cross-coupling reactions. For the details of reaction conditions, see J. Tsuji. Palladium Reagents and Catalysts: Innovations in Organic Synthesis; John wiley & Sons: Chichester, England, 1995, C. Wolf, et al. J. Org. Chem. (2003), 68, 7551-7554, special issue: 30 years of the cross-coupling reactions, J. Organomet. Chem., (2002), 653, G. Y. Li, J. Org. Chem. (2002), 67, 3643-3650, G. Y. Li, Angew. Chem. Intl. Ed., (2001), 1513-1516, each herein incorporated by reference with regard to such teaching.

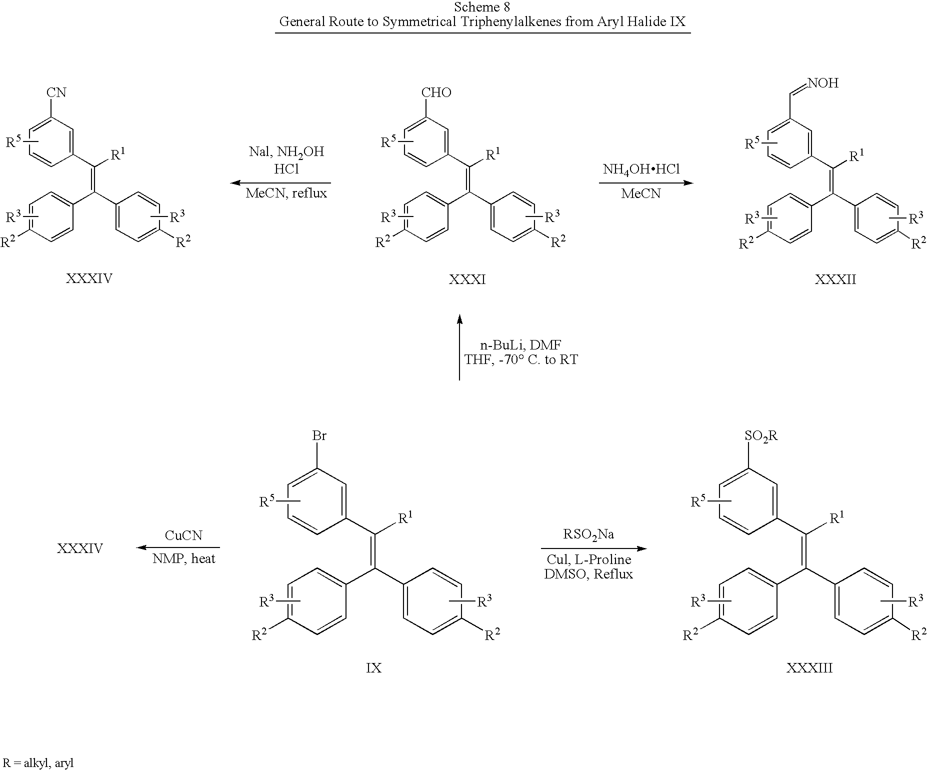

A variety of substituted symmetrical triphenylalkenes can be prepared by using the versatile intermediate XXXVII (Scheme 9). Compound XXXVII can be prepared from XXXVI by following the literature procedure described in the art. For reaction conditions, see M. Kodomari et al Tetrahedron Lett., (2001), 42, 3105 herein incorporated by reference with regard to such teaching. Tansition-metal catalysed cross coupling carbon-carbon bond forming reactions, described in the art, can be employed to make compound III from XXXVII.

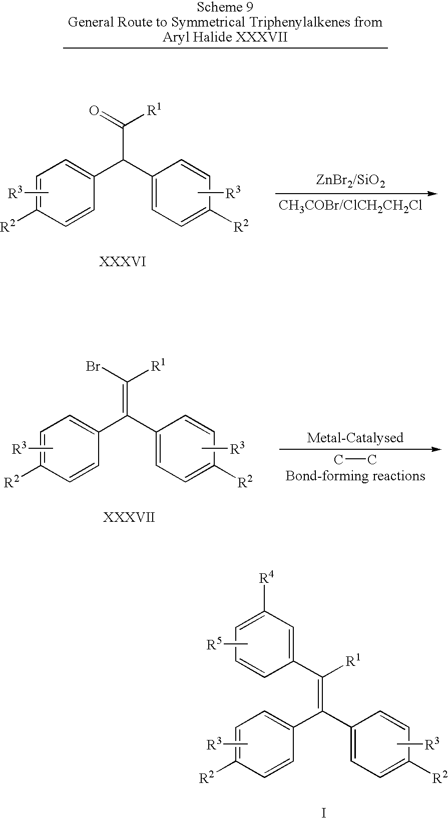

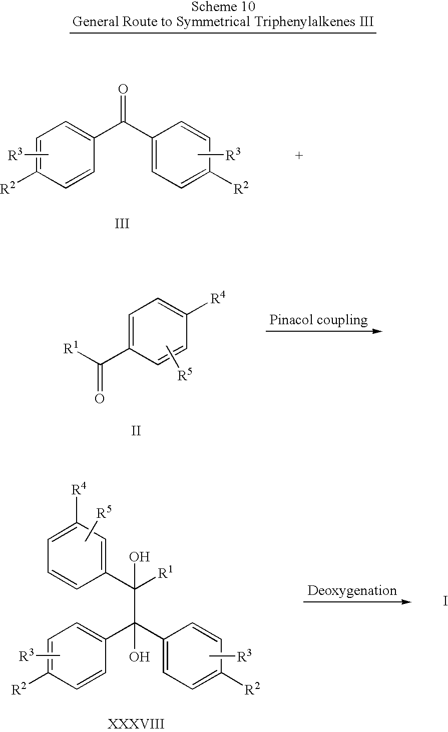

The triarylated compound I can also be prepared using the two step sequence as shown in scheme 10. Ketones II and III can be coupled using pinacol coupling method to give the vicinal diol XXXVIII. The diol compound XXXVIII can be transformed to olefin I using the deoxygenation conditions that are well documented in the art. For Pinacol coupling reaction conditions, see T. Wirth et al Angew. Chem. Int. Ed. Engl. (1996), 35, 61, X. Xu et al J. Org. Chem. (2005), 70, 8594 and leading references cited therein and for olefin synthesis by deoxygenation conditions, see E. Block in Organic Reactions (1984), 30, 457, herein incorporated by references with regard to such teaching.

### EXAMPLES

The following specific examples are included as illustrations and are not to be construed as limiting the scope of the present invention.

### Example 1 (3)

### (2E)-3-{3-[1-Ethyl-2,2-bis(4-hydroxyphenyl)ethenyl]phenyl}-2-propenoic acid (3)

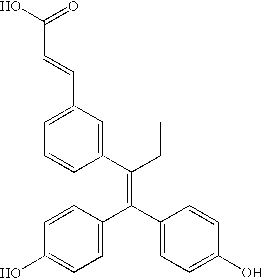

### Step 1: 4,4′-[2-(3-Bromophenyl)-1-butene-1,1-diyl]diphenol (1)

### General Procedure for McMurry Coupling:

To a stirred suspension of zinc powder (13.7 g, 0.211 mol) in THF (300 mL) was slowly added TiCl₄.2THF complex (35.0 g, 0.105 mol) via a funnel at room temperature under a nitrogen atmosphere. The reaction mixture was heated at reflux for 1.0 h. A solution of bis(4-hydroxyphenyl)methanone (4.5 g, 0.021 mol), and 1-(3-bromophenyl)-1-propanone (13.42 g, 0.063 mol) in THF (200 mL) was added to the reaction mixture. The reaction mixture was heated at reflux with stirring under a nitrogen atmosphere for an additional 2 h. The reaction mixture was allowed to cool at room temperature. A standard work-up of the McMurry coupling reaction is followed. As used herein, the term “standard work-up” refers generally to the standard work-up of the McMurry reaction as follows: The reaction mixture was poured into a 20% aqueous HCl (400 mL) slowly and stirred for 0.5 h. The reaction mixture was extracted with EtOAc (4×250 mL). The combined organic phase was washed with brine (2×100 mL), dried over Na₂SO₄, filtered, and the filtrate concentrated under reduced pressure to give the crude product as a gold-yellow oil. The crude product was purified by flash chromatography over SiO₂ with hexanes:ethyl acetate (100:0 to 3:2) as an eluent to afford a white foam. The white foam was recrystallized from acetonitrile to afford 7.960 g (96%) of the title compound 1 as a white crystalline solid. ¹H NMR (400 MHz, DMSO-d₆): δ 0.82 (t, J=7.2 Hz, 3H), 2.38 (q, J₁=14.8 Hz, J₂=7.6 Hz, 2H), 6.42 (d, J=8.8 Hz, 2H), 6.59 (d, J=8.4 Hz, 2H), 6.72 (d, J=8.4 Hz, 2H), 6.95 (d, J=8.4 Hz, 2H), 7.07 (dd, J₁=16.4 Hz, J₂=7.6 Hz, 1H), 7.11 (dd, J₁=15.2 Hz, J₂=7.6 Hz, 1H), 7.23 (br s, 1H), 7.25 (br d, J=8.0 Hz, 1H), 9.21 (s, 1H), 9.41 (s, 1H). LCMS (ESI): m/z 395 (M−H)⁻.

### Step 2: Ethyl (2E)-3-{3-[1-ethyl-2,2-bis(4-hydroxyphenyl)ethenyl]phenyl}-2-propionate (2)

To a round bottom flask were added 4,4′-[2-(3-bromophenyl)-1-butene-1,1-diyl]diphenol

1 (0.791 g, 2 mmol), ethyl acrylate (2.16 mL, 20 mmol.), dichlorobis(triphenylphosphine) palladium(II) (0.140 g, 0.2 mmol, 10 mol %), triethylamine (1.4 mL, 10 mmol), and DMF (10 mL). The stirred reaction mixture was heated to 110° C. for 9 h under a nitrogen atmosphere. The reaction mixture was allowed to cool at room temperature and then diluted with EtOAc (200 mL). The reaction mixture was washed with water (2×50 mL), brine (1×40 mL), dried (Na₂SO₄), filtered, and then concentrated under reduced pressure to afford the crude product as an oil. The crude product was purified by flash chromatography over SiO₂ with hexanes:ethyl acetate (19:1 to 1:1) as an eluent to give 0.590 g (71%) of the title compound 2 as a pale yellow foam. ¹H NMR (300 MHz, DMSO-d₆): δ 0.85 (t, J=7.4 Hz, 3H), 1.25 (t, J=7.0 Hz, 3H), 2.46 (q, J₁=14.4 Hz, J₂=7.2 Hz, 2H), 4.17 (q, J₁=14.0 Hz, J₂=7.0 Hz, 2H), 6.41 (d, J=8.4 Hz, 2H), 6.51 (d, J=15.9 Hz, 1H), 6.62 (d, J=9.0 Hz, 2H), 6.75 (d, J=8.4 Hz, 2H), 6.99 (d, J=8.4 Hz, 2H), 7.08 (br d, J=7.8 Hz, 1H), 7.20 (dd, J₁=15.9 Hz, J₂=7.8 Hz, 1H), 7.45 (br d, J=6.3 Hz, 1H), 7.46 (br s, 1H), 7.25 (d, J=16.2 Hz, 1H), 9.16 (s, 1H), 9.41 (s, 1H). LCMS (ESI): m/z 413. (M−H)⁻.

### Step 3: (2E)-3-{3-[1-Ethyl-2,2-bis(4-hydroxyphenyl)ethenyl]phenyl}-2-propenoic acid (3)

A standard work-up of ester saponification is followed. As used herein, the term “standard ester saponification” refers to the standard saponification of an ester as follows: To a stirred solution of ethyl (2E)-3-{3-[1-ethyl-2,2-bis(4-hydroxyphenyl)ethenyl]phenyl}-2-propenoate 2 (0.400 g, 0.965 mmol) in THF (7 mL) and EtOH (7 mL) was slowly added 1 N NaOH (5 mL) solution at room temperature. The reaction mixture was heated to 70° C. and stirred at that temperature for 1.5 h. Reaction mixture was cooled to room temperature and then poured into 20% aqueous HCl (50 mL). The product was separated out as an off-white solid. The suspension was filtered and the filtered solid was dried to afford 0.310 g (86%) of the title compound 3 as an off-white solid. mp 280-281° C., ¹H NMR (300 MHz, DMSO-d₆): δ 0.86 (t, J=7.4 Hz, 3H), 2.44 (q, J₁=14.4 Hz, J₂=7.2 Hz, 2H), 6.39 (d, J=16.6 Hz, 1H), 6.41 (d, J=8.4 Hz, 2H), 6.62 (d, J=8.4 Hz, 2H), 6.75 (d, J=8.4 Hz, 2H), 6.99 (d, J=8.4 Hz, 2H), 7.08 (br d, J=7.8 Hz, 1H), 7.20 (dd, J₁=15.6 Hz, J₂=8.1 Hz, 1H), 7.41 (br d, J=4.5 Hz, 1H), 7.42 (br s, 1H), 7.47 (d, J=16.2 Hz, 1H), 9.16 (s, 1H), 9.41 (s, 1H), 12.35 (br s, 1H). LCMS (ESI): m/z 385 (M−H)⁻.

### Example 2 (6)

### (2E)-3-{5-[1-Ethyl-2,2-bis(4-hydroxyphenyl)ethenyl]-2-fluorophenyl}-2-propenoic acid (6)

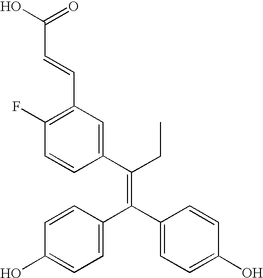

### Step 1: 4,4′-[2-(3-Bromo-4-fluorophenyl)-1-butene-1,1-diyl]diphenol (4)

The general McMurry procedure described for 1 was employed using bis(4-hydroxyphenyl)methanone (3.60 g, 0.0168 mol) and 1-(3-bromo-4-fluorophenyl)-1-propanone (11.65 g, 0.0504 mol). Standard work-up followed by purification gave 6.420 g (93%) of the title compound 4 as an off-white solid. ¹H NMR (400 MHz, DMSO-d₆): δ 0.83 (t, J=7.2 Hz, 3H), 2.38 (q, J=7.6 Hz, 2H), 6.44 (d, J=8.4 Hz, 2H), 6.59 (d, J=8.4 Hz, 2H), 6.72 (d, J=8.4 Hz, 2H), 6.95 (d, J=8.8 Hz, 2H), 7.09-7.05 (m, 1H), 7.15 (app. t, J=8.8 Hz, 1H), 7.36 and 7.34 (dd, J₁=7.2 Hz, J₂=2.4 Hz, 1H), 9.38 (s, 1H), 9.41 (s, 1H). LCMS (APCI): m/z 413 (M−H)⁻.

### Step 2: 1,1-Dimethylethyl (2E)-3-{5-[1-ethyl-2,2-bis(4-hydroxyphenyl)ethenyl]-2-fluorophenyl}-2-propenoate (5)

In a 10 mL glass tube were placed 4,4′-[2-(3-bromo-4-fluorophenyl)-1-butene-1,1-diyl]diphenol 4 (0.414 g, 1 mmol), PdCl₂(Ph₃P)₂ (0.071 g, 0.1 mmol), triethylamine (0.692 mL, 5 mmol), ethylacrylate (1.0 ml, ˜10 mmol), DMF (4 mL), and a stir bar under nitrogen atmosphere. The vessel was sealed with a septum and irradiated for 20 min at 200° C. in a smith synthesizer microwave. The reaction mixture was cooled rapidly to room temperature. The reaction mixture was transferred to a separatory funnel, diluted with sat. NaHCO₃ (50 mL), and water (50 mL), and the mixture extracted with EtOAc (3×50 mL). The combined organic layer was washed with brine (1×30 mL), dried (Na₂SO₄), filtered and the filtrate concentrated under reduced pressure to afford a crude product which was purified using SiO₂ column chromatography to afford 0.385 g (89%) of the title compound 5 as an off-white foam. ¹H NMR (400 MHz, DMSO-d₆): δ 0.83 (t, J=7.6 Hz, 3H), 1.23 (t, J=7.2 Hz, 3H), 2.44 (q, J₁=14.8 Hz, J₂=7.2 Hz, 2H), 4.16 (q, J₁=14.4 Hz, J₂=7.2 Hz, 2H), 6.41 (d, J=8.40 Hz, 2H), 6.52 (d, J=16.4 Hz, 1H), 6.60 (d, J=8.4 Hz, 2H), 6.73 (d, J=8.4 Hz, 2H), 6.96 (d, J=8.8 Hz, 2H), 7.07-7.02 (m, 2H), 7.58 (d, J=16.0 Hz, 1H), 7.59 (app. br d, J=7.6 Hz, 1H), 9.17 (s, 1H), 9.14 (s, 1H). LCMS (APCI): m/z 455 (M+Na) 1 and 433 (M+H)⁺.

### Step 3: (2E)-3-{5-[1-Ethyl-2,2-bis(4-hydroxyphenyl)ethenyl]-2-fluorophenyl}-2-propenoic acid (6)

To a stirred solution of 1,1-dimethylethyl (2E)-3-{5-[1-ethyl-2,2-bis(4-hydroxyphenyl)ethenyl]-2-fluorophenyl}-2-propenoate 5 (0.320 g, 0.74 mmol) in THF (20 mL) and EtOH (20 mL) was slowly added 1 N NaOH (10 mL) solution at room temperature. The reaction mixture was stirred at 70° C. for 1 h. The reaction mixture was cooled to room temperature and then poured into 20% aqueous HCl (50 mL). Standard work-up followed by purification gave 0.260 g (87%) of the title compound 6 as an off-white solid. ¹H NMR (400 MHz, DMSO-d₆): δ 0.83 (t, J=7.6 Hz, 3H), 2.42 (q, J₁=14.8 Hz, J₂=7.2 Hz, 2H), 6.41 (d, J=16.8 Hz, 1H), 6.42 (d, J=8.4 Hz, 2H), 6.60 (d, J=8.4 Hz, 2H), 6.72 (d, J=8.4 Hz, 2H), 6.96 (d, J=8.4 Hz, 2H), 7.06-7.02 (m, 2H), 7.51 (d, J=16.4 Hz, 1H), 7.53 (br dd, J₁=5.6 Hz, J₂=1.6 Hz, 1H), 9.4-9.02 (br, 2H), 12.5 (br s, 1H). LCMS (ESI): m/z 403 (M−H)⁻.

### Example 3 (7)

### 4,4′-{2-[3-(3,5-Dimethyl-4-isoxazolyl)phenyl]-1-butene-1,1-diyl}diphenol (7)

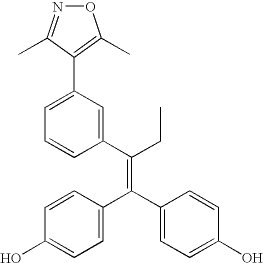

### 4,4′-{2-[3-(3,5-Dimethyl-4-isoxazolyl)phenyl]-1-butene-1,1-diyl}diphenol (7)

A round-bottom flask was charged with 4,4′-[2-(3-bromophenyl)-1-butene-1,1-diyl]diphenol 1 (0.200 g, 0.51 mmol), PdCl₂(PPh₃)₂, (0.024 g, 0.034 mmol), 3,5-dimethyl-4-isoxazolyl)boronic acid (0.090 g, 0.64 mmol), aqueous 2 M Na₂CO₃, (0.106 g, 0.5 mL, 1.0 mmol), THF (4 mL), and water (1 mL) under a nitrogen atmosphere. The reaction mixture was refluxed for 12 h. The reaction mixture was cooled to room temperature, diluted with EtOAc (100 mL), washed with H₂O, brine, dried, and concentrated under reduced pressure to afford the crude product. The product was purified by SiO₂ chromatography using hexanes:EtOAc (19:1 to 1:1) as an eluent to give 0.100 g (48%) of the title compound 7 as a white solid. mp 173-174° C. ¹H NMR (400 MHz, DMSO-d₆): δ 0.85 (t, J=5.8 Hz, 3H), 1.97 (s, 3H), 2.12 (s, 3H), 2.41 (q, J=14.4 Hz, J₂=7.2 Hz, 2H), 6.40 (d, J=8.4 Hz, 2H), 6.61 (d, J=8.4 Hz, 2H), 6.72 (d, J=8.4 Hz, 2H), 6.94 (d, J=5.2 Hz, 1H), 6.96 (d, J=8.4 Hz, 1H), 7.08 (br d, J=7.6 Hz, 1H), 7.19 (br d, J=7.6 Hz, 1H), 7.31 (dd, J₁=15.6 Hz, J₂=8.0 Hz, 1H), 9.15 (s, 3H), 9.39 (s, 3H). LCMS (APCI): m/z 411 (M−H)⁻.

### Example 4 (8)

### 4,4′-{2-[3-(3,5-Dimethyl-4-isoxazolyl)-4-fluorophenyl]-1-butene-1,1-diyl}diphenol (8)

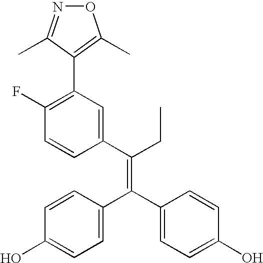

### 4,4′-{2-[3-(3,5-Dimethyl-4-isoxazolyl)-4-fluorophenyl]-1-butene-1,1-diyl}diphenol (8)

The Suzuki coupling procedure described for 7 was employed using 4,4′-[2-(3-bromo-4-fluorophenyl)-1-butene-1,1-diyl]diphenol 4 (0.413 g, 1.0 mmol) and 3,5-dimethyl-4-isoxazolyl)boronic acid (0.423 g, 3 mmol) in DME (10 mL). The standard work-up followed by purification gave 0.396 g (92%) of the title compound 8 as an off-white solid. ¹H NMR (400 MHz, DMSO-d₈): δ 0.86 (t, J=7.6 Hz, 3H), 1.91 (s, 3H), 2.07 (s, 3H), 2.40 (q, J=7.2 Hz, 2H), 6.43 (d, J=8.4 Hz, 2H), 6.61 (d, J=8.8 Hz, 2H), 6.72 (d, J=8.4 Hz, 2H), 6.93 (app. m, 1H), 6.95 (d, J=8.4 Hz, 2H), 7.18 (app. t, J=8.8 Hz, 1H), 7.27-7.23 (m, 1H), 9.18 (s, 1H), 9.39 (s, 1H). LCMS (ESI): m/z 428 (M−H)⁻ and 430 (M+H)⁺.

### Example 5 (9)

### 4,4′-{2-[3-(3-Furanyl)1-butene-1,1-diyl}diphenol (9)

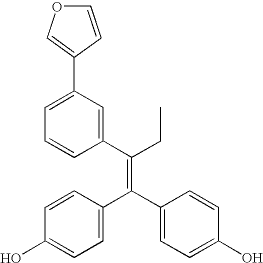

### 4,4′-{2-[3-(3-Furanyl)1-butene-1,1-diyl}diphenol (9)

The general Suzuki coupling procedure described for 7 was employed using 4,4′-[2-(3-bromophenyl)-1-butene-1,1-diyl]diphenol 1 (1.58 g, 4.0 mmol) and 3-furanylboronic acid (1.344 g, 12 mmol). Standard work-up followed by purification gave 1.150 g (75%) of the title compound 9 as an off-white solid. ¹H NMR (400 MHz, DMSO-d₆): δ 0.85 (t, J=7.2 Hz, 3H), 2.45 (q, J=7.2 Hz, 2H), 6.39 (d, J=8.8 Hz, 2H), 6.62 (d, J=8.8 Hz, 2H), 6.73 (d, J=8.4 Hz, 2H), 6.79 (s, 1H), 6.89 (d, J=7.6 Hz, 1H), 6.96 (d, J=8.4 Hz, 1H), 7.13 (app. t, J=8.0 Hz, 1H), 7.30 (d, J=7.6 Hz, 1H), 7.35 (s, 1H), 7.68 (t, J=1.6 Hz, 1H), 8.02 (s, 1H), 9.13 (s, 1H), 9.39 (s, 1H). LCMS (APCI): m/z 383 (M+H)⁺.

### Example 6 (10)

### 4,4′-{2-[4-Fluoro-3-(3-turanyl)phenyl]-1-butene-1,1-diyl}diphenol (10)

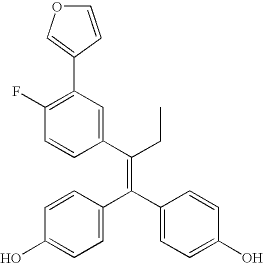

### 4,4′-{2-[4-Fluoro-3-(3-furanyl)phenyl]-1-butene-1,1-diyl}diphenol (10)

The Suzuki coupling procedure described for 7 was employed using 4,4′-[2-(3-bromo-4-fluorophenyl)-1-butene-1,1-diyl]diphenol 4 (0.250 g, 0.61 mmol) and 3-furanylboronic acid (0.205 g, 1.83 mmol) in DME (5 mL). Standard work-up followed by purification gave 0.205 g (85%) of the title compound 10 as an off-white solid. ¹H NMR (400 MHz, DMSO-d₆): δ 0.85 (t, J=7.2 Hz, 3H), 2.44 (q, J=7.6 Hz, 2H), 6.42 (d, J=8.8 Hz, 2H), 6.63 (d, J=8.8 Hz, 2H), 6.73 (d, J=8.4 Hz, 2H), 6.80 (br s, 1H), 6.95-6.91 (m, 1H), 6.96 (d, J=8.4 Hz, 2H), 7.05 and 7.02 (dd, J₁=11.2 Hz, J₂=8.4 Hz, 1H), 7.42 and 7.40 (dd, J₁=7.6 Hz, J₂=2.0 Hz, 1H), 7.73 (t, J=1.2 Hz, 1H), 7.97 (br s, 1H), 9.16 (s, 1H), 9.40 (s, 1H). LCMS (ESI): m/z 399 (M−H)⁻ and 401 (M+H)⁺.

### Example 7 (16)

### 4,4′-(2-{3-[(2-Hydroxyethyl)oxy]phenyl}-1-butene-1,1-diyl)diphenol (16)

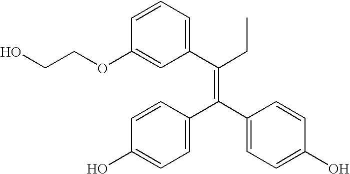

### Step 1: N-Methyl-N,3-bis(methyloxy)benzamide (11)

To a stirred solution of pyridine (14.2 mL, 0.176 mol) and N,O-dimethylhydroxylamine hydrochloride (10.72 g, 0.110 mol) in CH₂Cl₂ (100 mL) at 5° C. was added 3-(methyloxy)benzoyl chloride (15.0 g, 0.088 mol) in THF dropwise. The resultant reaction mixture was stirred at room temperature for 15 h. Reaction mixture was poured into H₂O (500 mL) and then extracted with EtOAc (4×150 mL). The combined organic layer was washed with brine (1×100 mL), dried (Na₂SO₄), and then concentrated under reduced pressure to afford 16.85 g (98%) of the title compound 11 as a liquid. ¹H NMR (400 MHz, CDCl₃): δ 3.32 (s, 3H), 3.55 (s, 3H), 3.81 (s, 3H), 6.96 and 6.98 (dd, J₁=8.4 Hz, J₂=2.4 Hz, 1H), 7.17 (br s, 1H), 7.21 (br d, J=7.6 Hz, 1H), 7.29 (dd, J₁=15.6 Hz, J₂=8.0 Hz, 1H).

### Step 2: 1-[3-(Methyloxy)phenyl]-1-propanone (12)

To a stirred solution of N-methyl-N,3-bis(methyloxy)benzamide 11 (9.0 g, 0.046 mol) in THF was added 3.0 M solution of ethyl magnesium bromide (23 mL, 0.069 mol) slowly at 5° C. The resultant reaction mixture was stirred at room temperature for 2.5 h. The reaction mixture was poured into 20% aqueous HCl (300 mL) and then extracted with EtOAc (3×150 mL). The combined organic layer was washed with brine (1×100 mL), dried, filtered, and concentrated under reduced pressure to afford 7.50 g (99%) of the title compound 12 as an oil. ¹H NMR (400 MHz, CDCl₃): δ 1.19 (t, J=7.2 Hz, 3H), 2.96 (q, J=7.2 Hz, 2H), 3.82 (s, 3H), 7.06 and 7.08 (dd, J₁=8.0 Hz, J₂=2.4 Hz, 1H), 7.33 (dd, J₁=15.2 Hz, J₂=8.0 Hz, 1H), 7.47 (dd, J₁=4.0 Hz, J₂=1.6 Hz, 1H), 7.52 (br d, J=7.6 Hz, 1H).

### Step 3: 1-(3-Hydroxyphenyl)-1-propanone (13)

To a stirred solution of 1-[3-(methyloxy)phenyl]-1-propanone 12 (6.5 g, 0.0396 mol) in toluene (100 mL) was slowly added aluminum chloride (8.0 g, 0.0594 mol) via a powder addition funnel under a nitrogen atmosphere at room temperature. The stirred reaction mixture was heated at reflux for 5 h under a nitrogen atmosphere. The reaction mixture was allowed to cool at room temperature and then poured into 10% aqueous HCl (200 mL). The reaction mixture was transferred to a separating funnel and the layers were separated. The aqueous phase was extracted with ethyl acetate (3×150 mL). The combined organic layer was washed with brine (1×50 mL), dried over Na₂SO₄, filtered, and the filtrate concentrated to a crude product which was purified by column chromatography to afford 5.580 g (94%) of the title compound 13 as a solid. ¹H NMR (400 MHz, CDCl₃): [symbol] 1.23 (t, J=7.6 Hz, 3H), 2.99 (q, J=7.2 Hz, 2H), 7.10 and 7.12 (dd, J₁=8.0 Hz, J₂=2.4 Hz, 1H), 7.32 (dd, J=8.0 Hz, J₂=8.0 Hz, 1H), 7.51 (br d, J=8.0 Hz, 1H), 7.62 (dd, J₁=2.4 Hz, 1H).

### Step 4: Ethyl[(3-propanoylphenyl)oxy]acetate (14)

A round bottom flask was charged with 1-(3-hydroxyphenyl)-1-propanone 13 (1.5 g, 10.0 mol), K₂CO₃ (4.15 g, 30.0 mmol), acetone (50 mL), and ethylbromoacetate (2.2 mL, 20.0 mmol) under N₂. The reaction mixture was reefluxed for 5 h and cooled to room temperature. The reaction mixture was filtered and the filtrate was concentrated under reduced pressure to afford the crude product. The product was purified by SiO₂ column chromatography using hexanes and EtOAc (19:1 to 4:1) as an eluent to give 2.3 g (98%) of the title compound 14 as an oil. ¹H NMR (400 MHz, CDCl₃): δ 1.21 (t, J=7.2 Hz, 3H), 1.29 (t, J=7.2 Hz, 3H), 2.98 (q, J=6.8 Hz, 2H), 4.27 (q, J=7.2 Hz, 2H), 4.67 (s, 2H), 7.11 and 7.13 (dd, J₁=8.4 Hz, J₂=2.4 Hz, 1H), 7.37 (dd, J₁=8.0 Hz, J₂=8.0 Hz, 1H), 7.48 (dd, J₁=1.6 Hz, J₂=1.6 Hz, 1H), 7.58 (br d, J=7.6 Hz, 1H).

### Step 5: Ethyl({3-[1-ethyl-2,2-bis(4-hydroxyphenyl)ethenyl]phenyl}oxy)acetate (15)

The general McMurry procedure described under 1 method was employed using bis(4-hydroxyphenyl)methanone (0.363 g, 1.69 mmol) and ethyl [(3-propanoylphenyl)oxy]acetate 14 (1.2 g, 5.08 mmol). Standard work-up followed by purification gave 0.672 g (95%) of the title compound 15 as an off-white foam. ¹H NMR (400 MHz, CDCl₃): δ 0.81 (t, J=7.2 Hz, 3H), 1.71 (t, J=7.2 Hz, 3H), 2.35 (q, J=7.2 Hz, 2H), 4.12 (q, J=6.8 Hz, 2H), 4.60 (s, 2H), 6.38 (d, J=8.4 Hz, 2H), 6.58 (d, J=8.4 Hz, 2H), 6.64-6.61 (br m, 3H), 6.94 (d, J=8.8 Hz, 2H), 7.04 (dd, J₁=7.6 Hz, J₂=7.6 Hz, 1H), 9.14 (s, 1H), 9.38 (s, 1H). LCMS (ESI): m/z 441 (M+Na)⁺.

### Step 6: 4,4′-(2-{3-[(2-Hydroxyethyl)oxy]phenyl}-1-butene-1,1-diyl)diphenol (16)

To a stirred solution of ethyl({3-[1-ethyl-2,2-bis(4-hydroxyphenyl)ethenyl]phenyl}oxy)acetate 15 (0.180 mg, 0.43 mmol) in THF (25 mL) was added 1 M solution of LiAlH₄ (1.3 mL, 1.29 mmol) under N₂ atmosphere at room temperature. The reaction mixture was stirred at RT for 0.5 h. The reaction mixture was quenched with EtOAc (5 mL) and stirred for an additional 10 min before pouring into 20% aqueous HCl (100 mL). The reaction mixture was extracted with EtOAc (4×50 mL). The combined organic layer was washed with brine (1×30 mL), dried over sodium sulfate and concentrated under reduced pressure and the crude product which was purified by flash column chromatography using CHCl₃ and MeOH (100:0 to 9:1) as an eluent to afford 136 mg (84%) of the title compound 16 as a white solid. ¹H NMR (400 MHz, DMSO-d₆): δ 0.82 (t, J=7.6 Hz, 3H), 2.35 (q, J=7.2 Hz, 2H), 3.62 (q, J₁=10.4 Hz, J₂=5.2 Hz, 2H), 3.79 (t, J=4.8 Hz, 2H), 4.80 (t, J=5.6 Hz, 1H), 6.39 (d, J_{—}8.8 Hz, 2H), 6.59 (d, J=8.8 Hz, 2H), 6.64-6.61 (br m, 3H), 6.71 (d, J=8.4 Hz, 2H), 6.94 (d, J=8.8 Hz, 2H), 7.04 (app. t, J=7.6 Hz, 1H), 9.13 (s, 1H), 9.37 (s, 1H). LCMS (ESI): m/z 399 (M+Na)⁺.

### Example 8 (17)

### ({3-[1-Ethyl-2,2-bis(4-hydroxyphenyl)ethenyl]phenyl}oxy)acetic acid (17)

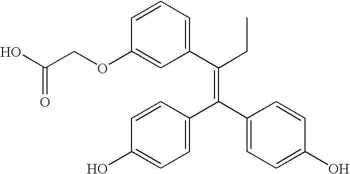

### ({3-[1-Ethyl-2,2-bis(4-hydroxyphenyl)ethenyl]phenyl}oxy)acetic acid (17)

ethyl({3-[1-ethyl-2,2-bis(4-hydroxyphenyl)ethenyl]phenyl}oxy)acetate 15 (0.050 g, 0.12 mmol) was dissolved in a mixture of THF and EtOH (1:1, 4 mL). To the stirred mixture was added 1 N NaOH (1 mL) and the reaction heated to 70° C. for 1 h. The reaction mixture was cooled to RT and poured into 50% aqueous HCl (30 mL), and then extracted with EtOAc (3×30 mL). The combined organic layer was washed with brine, dried, and filtered and the filtrate was concentrated under reduced pressure. The crude product was purified by flash column chromatography with chloroform and methanol (100:0 to 4:1) as an eluent to give 0.046 g (99%) of the title compound 17 as an off-white solid. ¹H NMR (400 MHz, DMSO-d₆): δ 0.81 (br t, 3H), 2.33 (app. br q, J=6.4 Hz, 2H), 4.05 (s, 2H), 6.40 (d, J=7.6 Hz, 2H), 6.54 (br d, J=6.4 Hz, 2H), 6.57 (br s, 3H), 6.93 (br d, J=8.0 Hz, 2H), 6.97 (app. br t, J=7.8 Hz, 1H), 9.34 (s, 1H), 9.60 (s, 1H). LCMS (ESI): m/z 413 (M+Na)⁺.

### Example 9 (20)

### 4-({3-[1-Ethyl-2,2-bis(4-hydroxyphenyl)ethenyl]phenyl}oxy)butanoic acid (20)

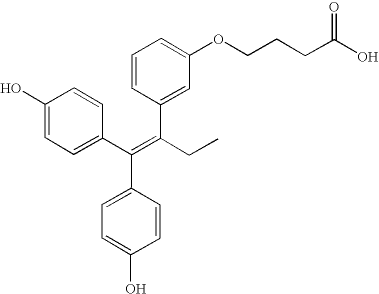

### Step 1: Ethyl 4-[(3-propanoylphenyl)oxy]butanoate (18)

A round bottom flask was charged with 1-(3-hydroxyphenyl)-1-propanone 13 (1.5 g, 10.0 mol), K₂CO₃ (4.15 g, 30.0 mmol), acetone (50 mL), and ethyl 4-bromobutanoate (2.87 mL, 20.0 mmol) under N₂. The reaction mixture was refluxed for 15 h and cooled to room temperature. The reaction mixture was filtered and the filtrate was concentrated under reduced pressure to afford the crude product. The product was purified by SiO₂ column chromatography using hexanes and EtOAc (19:1 to 4:1) as an eluent to give 2.42 g (92%) of the title compound 18 as an oil. ¹H NMR (400 MHz, CDCl₃): δ 1.21 (t, J=7.6 Hz, 3H), 1.25 (t, J=7.2 Hz, 3H), 2.11 (quintet, J=6.8 Hz, 2H), 2.51 (t, J=7.6 Hz, 2H), 2.97 (q, J=7.2 Hz, 2H), 4.05 (t, J=6.0 Hz, 2H), 4.14 (q, J=7.2 Hz, 2H), 7.06 and 7.08 (dd, J₁=8.4 Hz, J₂=2.8 Hz, 1H), 7.34 (app. t, J=8.0 Hz, 1H), 7.46 (app. br t, J=2.4 Hz, 1H), 7.52 (br d, J=7.6 Hz, 1H).

### Step 2: Ethyl 4-({3-[1-ethyl-2,2-bis(4-hydroxyphenyl)ethenyl]phenyl}oxy)butanoate (19)

The general McMurry procedure described for 1 was employed using bis(4-hydroxyphenyl)methanone (0.270 g, 1.26 mmol) and ethyl 4-[(3-propanoylphenyl)oxy]butanoate 18 (1.56 g, 3.78 mmol). Standard work-up followed by purification gave 0.672 g (95%) of the title compound 19 as an off-white foam. ¹H NMR (400 MHz, CDCl₃): δ 0.82 (t, J=7.6 Hz, 3H), 1.15 (t, J=7.2 Hz, 3H), 1.85 (app. quintet, J=6.8 Hz, 2H), 2.36 (q, J=7.2 Hz, 2H), 3.80 (t, J=6.4 Hz, 2H), 4.03 (q, J=6.8 Hz, 2H), 6.38 (d, J=8.4 Hz, 2H), 6.58 (d, J=8.4 Hz, 2H), 6.63-6.59 (m, 3H), 6.72 (d, J=8.4 Hz, 2H), J=8.4 Hz, 2H), 7.04 (app. t, J=7.6 Hz, 1H), 9.13 (s, 1H), 9.38 (s, 1H). LCMS (ESI): m/z 469 (M+Na)⁺.

### Step 3: 4-({3-[1-Ethyl-2,2-bis(4-hydroxyphenyl)ethenyl]phenyl}oxy)butanoic acid (20)

The general saponification procedure described for 3 (Example 1, Step 3) was employed using ethyl 4-({3-[1-ethyl-2,2-bis(4-hydroxyphenyl)ethenyl]phenyl}oxy)butanoate 19 (0.200 g, 0.448 mmol). Standard work-up followed by purification gave 0.186 g (99%) of the title compound 20 as an off-white solid. ¹H NMR (400 MHz, DMSO-d₆): δ 0.82 (t, J=7.2 Hz, 3H), 1.82 (app. quintet, J=7.2 Hz, 2H), 2.32 (t, J=7.2 Hz, 2H), 2.36 (q, J=6.8 Hz, 2H), 3.80 (t, J=6.8 Hz, 2H), 6.39 (d, J=8.8 Hz, 2H), 6.59 (d, J=8.4 Hz, 2H), 6.63-6.61 (br m, 3H), 6.72 (d, J=8.4 Hz, 2H), 6.94 (d, J=8.4 Hz, 2H), 7.03 (app, t, J=8.0 Hz, 1H). LCMS (ESI): m/z 419 (M+H)⁺.

### Example 10 (21)

### 4,4′-(2-{3-[(4-Hydroxybutyl)oxy]phenyl}-1-butene-1,1-diyl)diphenol (21)

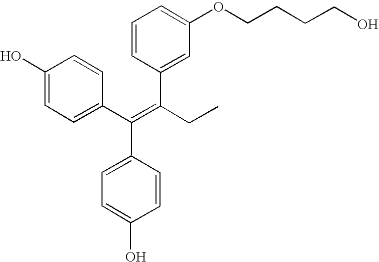

### 4,4′-(2-{3-[(4-Hydroxybutyl)oxy]phenyl}-1-butene-1,1-diyl)diphenol (1)

To a stirred solution of ethyl 4-({3-[1-ethyl-2,2-bis(4-hydroxyphenyl)ethenyl]phenyl}oxy)butanoate 19 (0.200 mg, 0.45 mmol) in THF (30 mL) was added 1 M solution of LiAlH₄ (2.2 mL, 2.24 mmol) under N₂ atmosphere at room temperature. The reaction mixture was stirred at RT for 0.5 h then quenched with EtOAc (5 mL) and stirred for an additional 10 min before pouring into 1 N aqueous HCl (100 mL). The reaction mixture was extracted with EtOAc (4×40 mL). The combined organic layer was washed with brine (1×30 mL), dried over sodium sulfate and concentrated under reduced pressure. The crude product was purified by flash column chromatography using CHCl₃ and MeOH (100:0 to 9:1) as an eluent to afford 176 mg (97%) of the title compound 21 as an off-white solid. ¹H NMR (400 MHz, DMSO-d₆): δ 0.82 (t, J=7.2 Hz, 2H), 1.49 (app. quintet, J=7.6 Hz, 2H), 1.62 (app. quintet, J=7.6 Hz, 2H), 2.35 (q, J=7.2 Hz, 2H), 3.40 (q, J=5.6 Hz, 2H), 3.77 (t, J=6.4 Hz, 2H), 4.41 (t, J=4.8 Hz, 1H), 6.39 (d, J=8.4 Hz, 2H), 6.59 (d, J=8.4 Hz, 2H), 6.63 and 6.60 (br s, 3H), 6.72 (d, J=8.4 Hz, 2H), 6.94 (d, J=8.4 Hz, 2H), 7.04 (app. t, J=7.6 Hz, 1H). LCMS (ESI): m/z 427 (M+Na)⁺.

### Example 11 (23)

### 4,4′-{2-[3-({2-[(2-Hydroxyethyl)oxy]ethyl}oxy)phenyl]-1-butene-1,1-diyl}diphenol (23)

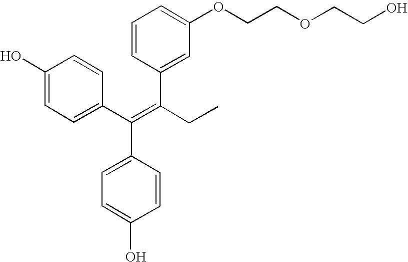

### Step 1: 1-[3-({2-[(2-Hydroxyethyl)oxy]ethyl}oxy)phenyl]-1-propanone 22)

A round bottom flask was charged with 1-(3-hydroxyphenyl)-1-propanone 13 (1.5 g, 10.0 mol), K₂CO₃ (4.15 g, 30.0 mmol), acetone (50 mL), and 2-[(2-chloroethyl)oxy]ethanol (2.60 mL, 25.0 mmol) under N₂. The reaction mixture was refluxed for 60 h and cooled to room temperature, filtered, and the filtrate concentrated under reduced pressure. The crude product was purified by SiO₂ column chromatography using hexanes and EtOAc (19:1 to 1:1) as an eluent to give 2.26 g (95%) of the title compound 22 as colorless oil. ¹H NMR (400 MHz, CDCl₃): δ1.19 (t, J=7.2 Hz, 3H), 2.32 (br d, J=13.6 Hz, 1H), 2.96 (q, J=7.2 Hz, 2H), 3.66 (t, J=4.4 Hz, 2H), 3.75 (t, J=4.4 Hz, 2H), 3.86 (t, J=4.4 Hz, 2H), 4.18 (t, J=5.2 Hz, 2H), 7.11 and 7.09 (dd, J₁=8.4 Hz, J₂=2.4 Hz, 1H), 7.34 (app. t, J=7.6 Hz, 1H), 7.51 (br s, 1H), 7.53 (d, J=7.6 Hz, 1H). LCMS (ESI): m/z 267.13 (M+H)⁺.

### Step 2: 4,4′-{2-[3-({2-[(2-Hydroxyethyl)oxy]ethyl}oxy)phenyl]-1-butene-1,1-diyl}diphenol (23)

The general McMurry procedure described for 1 was employed using bis(4-hydroxyphenyl)methanone (0.180 g, 0.84 mmol) and 1-[3-({2-[(2-hydroxyethyl)oxy]ethyl}oxy)phenyl]-1-propanone 22 (0.600 g, 2.52 mmol). Standard work-up followed by purification gave 0.278 g (79%) of the title compound 23 as an off-white foam. ¹H NMR (400 MHz, DMSO-d₆): δ 0.82 (t, J=7.6 Hz, 3H), 2.35 (t, J=7.6 Hz, 2H), 3.42 (t, J=4.8 Hz, 2H), 3.47 (t, J=4.8 Hz, 2H), 3.62 (t, J=4.4 Hz, 2H), 3.91 (t, J=5.2 Hz, 2H), 4.6 (t, J=5.6 Hz, 1H), 6.39 (d, J=8.8 Hz, 2H), 6.59 (d, J=8.4 Hz, 2H), 6.65-6.61 (m, 3H), 6.71 (d, J=8.4 Hz, 2H), 6.94 (d, J=8.4 Hz, 2H), 7.05 (app. t, J=7.6 Hz, 1H), 9.14 (s, 1H), 9.37 (s, 1H). LCMS (ESI): m/z 421 (M+H)⁺.

### Example 12 (28)

### 4,4′-(2-{3-[(2-Hydroxyethyl)oxy]phenyl}-1-pentene-1,1-diyl)diphenol (28)

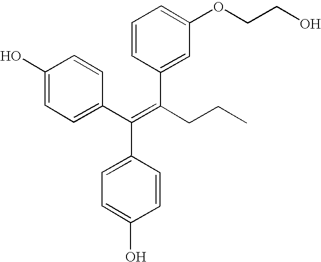

### Step 1: 1-[3-(Methyloxy)phenyl]-1-butanone (24)

To a stirred solution of N-methyl-N,3-bis(methyloxy)benzamide 11 (12.50 g, 64.03 mmol) in THF was added 2.0 M solution of n-propyl magnesium chloride (48 mL, 96.05 mol) slowly at 5° C. The resultant reaction mixture was allowed to stir at room temperature for 6 h. The reaction mixture was poured into 1 N HCl (300 mL) and then extracted with EtOAc (4×200 mL). The combined organic layer was washed with brine (1×100 mL), dried, filtered, and concentrated under reduced pressure to afford 9.6 g (84%) of the title compound 24 as an oil. ¹H NMR (400 MHz, CDCl₃): δ 0.97 (t, J=7.2 Hz, 3H), 1.73 (app. sextet, J=7.2 Hz, 2H), 2.89 (t, J=7.2 Hz, 2H), 3.81 (s, 3H), 7.07 and 7.05 (dd, J₁=8.0 Hz, J₂=2.4 Hz, 1H), 7.32 (t, J=8.0 Hz, 1H), 7.46 (app. t, J=1.6 Hz, 1H), 7.50 (br d, J=7.6 Hz, 1H).

### Step 2: 1-(3-Hydroxyphenyl)-1-butanone (25)

The general demethylation procedure described for 13 was employed using 1-[3-(methyloxy)phenyl]-1-butanone (8.0 g, 45 mmol) and aluminum chloride (17.93 g, 135 mmol). Standard work-up followed by purification gave 7.150 g (97%) of the title compound 25 as an off-white solid. ¹H NMR (400 MHz, DMSO-d₆): δ 0.88 (t, J=7.6 Hz, 3H), 1.59 (app. sextet, J=7.2 Hz, 2H), 2.89 (t, J=7.2 Hz, 2H), 6.99 and 6.97 (dd, J₁=8.0 Hz, J₂=2.4 Hz, 1H), 7.30-7.26 (m, 2H), 7.37 (br d, J=8.0 Hz, 1H), 9.72 (s, 1H).

### Step 3: Ethyl[(3-butanoylphenyl)oxy]acetate (26)

A round bottom flask was charged with 1-(3-hydroxyphenyl)-1-butanone 25 (1.5 g, 9.14 mol), K₂CO₃ (3.79 g, 27.42 mmol), acetone (30 mL), and ethylbromoacetate (2.53 mL, 23.0 mmol) under N₂. The reaction mixture was refluxed for 15 h and cooled to room temperature. The reaction mixture was filtered and the filtrate concentrated under reduced pressure. The crude product was purified by SiO₂ column chromatography using hexanes and EtOAc (19:1 to 4:1) as an eluent to give 2.1 g (87%) of the title compound 26 as an oil. ¹H NMR (400 MHz, CDCl₃): δ 0.97 (t, J=7.6 Hz, 3H), 1.27 (t, J=7.2 Hz, 3H), 1.73 (sextet, J=3.2 Hz, 2H), 2.88 (t, J=7.6 Hz, 2H), 4.24 (q, J=7.2 Hz, 2H), 4.64 (s, 2H), 7.10 (m, 1H), 7.37-7.32 (m, 1H), 7.45 (br s, 1H), 7.54 (br d, J=7.2 Hz, 1H).

### Step 4: Ethyl({3-[2,2-bis(4-hydroxyphenyl)-1-propylethenyl]phenyl}oxy)acetate (27)

The general McMurry procedure described for 1 was employed using bis(4-hydroxyphenyl)methanone (0.570 g, 2.66 mmol) and ethyl [(3-butanoylphenyl)oxy]acetate 26 (2.0 g, 8.0 mmol). Standard work-up followed by purification gave 0.978 g (85%) of the title compound 27 as an off-white foam. ¹H NMR (400 MHz, CDCl₃): δ 0.73 (t, J=7.2 Hz, 3H), 1.23-1.16 (m, 5H), 2.30 (app. t, J=7.2 Hz, 2H), 4.12 (q, J=6.8 Hz, 2H), 4.59 (s, 2H), 6.38 (d, J=8.8 Hz, 2H), 6.59 (d, J=8.8 Hz, 2H), 6.64-6.61 (br m, 3H), 6.72 (d, J=8.4 Hz, 2H), 6.93 (d, J=8.4 Hz, 1H), 7.05 and 7.03 (dd, J₁=8.8 Hz, J₂=7.6 Hz, 1H), 9.12 (s, 1H), 9.36 (s, 1H). LCMS (ESI): m/z 455 (M+Na)⁺ and 433 (M+H)⁺.

### Step 5: 4,4′-(2-{3-[(2-Hydroxyethyl)oxy]phenyl}-1-pentene-1,1-diyl)diphenol (28)

To a stirred solution of ethyl({3-[2,2-bis(4-hydroxyphenyl)-1-propylethenyl]phenyl}oxy)acetate 27 (0.190 g, 0.44 mmol) in THF (30 mL) was added 1 M solution of LiAlH₄ (1.6 mL, 1.6 mmol) under N₂ atmosphere at room temperature. Standard work-up described for 16 and purification gave 130 mg (76%) of the title compound 28 as an off-white solid. ¹H NMR (400 MHz, DMSO-d₆): δ 0.73 (t, J=7.2 Hz, 3H), 1.21 (app. sextet, J=8.0 Hz, 2H), 2.30 (app. t, J=2.0 Hz, 2H), 3.62 (q, J=5.2 Hz, 2H), 3.79 (t, J=4.8 Hz, 2H), 4.79 (t, J=5.2 Hz, 1H), 6.39 (d, J=8.8 Hz, 2H), 6.63-6.59 (m, 5H), 6.71 (d, J=8.4 Hz, 2H), 6.94 (d, J=8.8 Hz, 2H), 7.03 (app. t, J=8.0 Hz, 1H), 9.12 (s, 1H), 9.36 (s, 1H). LCMS (ESI): m/z 413.09 (M+Na)⁺ and 391 (M+H)⁺.

### Example 13 (31)

### 4,4′-(2-{3-[(4-Hydroxybutyl)oxy]phenyl}-1-pentene-1,1-diyl)diphenol (31)

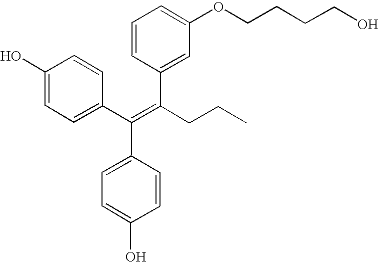

### Step 1: Ethyl 4-[(3-butanoylphenyl)oxy]butanoate (29)

A round bottom flask was charged with 1-(3-hydroxyphenyl)-1-butanone 25 (1.5 g, 9.14 mol), K₂CO₃ (3.79 g, 27.42 mmol), acetone (30 mL), and ethyl 4-bromobutanoate (3.3 mL, 23 mmol) under N₂. The reaction mixture was refluxed for 15 h then cooled to RT, filtered, and the filtrate was concentrated to give the crude product, which was purified to by SiO₂ column chromatography to afford 2.21 g (90%) of the title compound 29 as an oil. ¹H NMR (400 MHz, CDCl₃): δ 97 (t, J=7.2 Hz, 3H), 1.23 (t, J=6.8 Hz, 3H), 1.73 (app. sextet, J=7.2 Hz, 2H), 2.10 (app. pentet, J=6.8 Hz, 2H), 2.49 (q, J=7.2 Hz, 2H), 2.89 (t, J=7.2 Hz, 2H), 4.03 (t, J=6.0 Hz, 2H), 4.12 (q, J=6.8 Hz, 2H), 7.05 (br d, J=8.4 Hz, 1H), 7.32 (app. t, J=8.0 Hz, 1H), 7.44 (br s, 1H), 7.50 (d, J=7.6 Hz, 1H).

### Step 2: Ethyl 4-({3-[2,2-bis(4-hydroxyphenyl)-1-propylethenyl]phenyl}oxy) butanoate (30)

The general McMurry procedure described for 1 was employed using bis(4-hydroxyphenyl)methanone (0.564 g, 2.63 mmol) and ethyl 4-[(3-butanoylphenyl)oxy]butanoate 29 (2.20 g, 7.90 mmol). Standard work-up followed by purification gave 1.065 g (88%) of the title compound 30 as an off-white foam. ¹H NMR (400 MHz, DMSO-d₆): δ 0.73 (t, J=7.2 Hz, 3H), 1.15 (t, J=7.2 Hz, 3H), 1.24-1.18 (app. sextet, J=7.6 Hz, 2H), 1.85 (app. quintet, J=6.8 Hz, 2H), 2.29 (app. t, J=6.0 Hz, 2H), 2.37 (t, J=7.2 Hz, 2H), 3.80 (t, J=6.4 Hz, 2H), 4.04 (q, J=7.2 Hz, 2H), 6.38 (d, J=8.8 Hz, 2H), 6.63-6.58 (m, 5H), 6.71 (d, J=8.8 Hz, 2H), 6.93 (d, J=8.4 Hz, 2H), 7.03 (t, 8.0 Hz, 1H), 9.12 (s, 1H), 9.36 (s, 1H). LCMS (ESI): m/z 459 (M−H)⁻.

### Step 3: 4,4′-(2-{3-[(4-Hydroxybutyl)oxy]phenyl}-1-pentene-1,1-diyl)diphenol (31)

The general LAH reduction described for 16 was employed with ethyl 4-({3-[2,2-bis(4-hydroxyphenyl)-1-propylethenyl]phenyl}oxy)butanoate 30 (0.200 g, 0.434 mmol) and a 1 N LiAlH₄ (1.3 mL) in THF (50 mL) at RT. Standard work-up followed by purification gave 0.156 g (86%) of the title compound 31 as an off-white solid. ¹H NMR (400 MHz, DMSO-d₆): δ 0.73 (t, J=7.6 Hz, 3H), 1.21 (sextet, J=8.0 Hz, 2H), 1.47 (app. quintet, J=3.2 Hz, 2H), 1.62 (app. quintet, J=8.4 Hz, 2H), 2.30 (app. t, J=8.0 Hz, 2H), 3.36 (q, J=5.2 Hz, 2H), 3.77 (t, J=6.4 Hz, 2H), 4.40 (t, J=5.2 Hz, 1H), 6.38 (d, J=8.4 Hz, 2H), 6.62-6.58 (m, 5H), 6.71 (d, J=8.4 Hz, 2H), 6.93 (d, J=8.4 Hz, 2H), 7.03 (t, J=7.6 Hz, 1H), 9.12 (s, 1H), 9.36 (s, 1H). LCMS (ESI): m/z 417 (M−H)⁻.

### Example 14 (32)

### 4-({3-[2,2-Bis(4-Hydroxyphenyl)-1-propylethenyl]phenyl}oxy)butanoic acid (32)

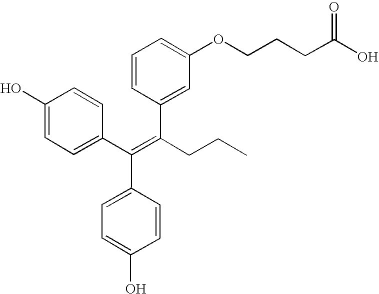

### 4-({3-[2,2-bis(4-Hydroxyphenyl)-1-propylethenyl]phenyl}oxy)butanoic acid (32)

The general saponification procedure described for 18 was followed using ethyl 4-({3-[2,2-bis(4-hydroxyphenyl)-1-propylethenyl]phenyl}oxy) butanoate 30 (0.170 g, 0.369 mmol) and a 1 N NaOH solution. Standard work-up followed by purification gave 0.146 g (91%) of the title compound 32 as a pale yellow solid. ¹H NMR (400 MHz, DMSO-d₆): δ 0.73 (t, J=7.6 Hz, 3H), 1.21 (app. sextet, J=8.0 Hz, 2H), 1.82 (app. quintet, J=6.8 Hz, 2H), 2.30 (t, J=7.6 Hz, 4H), 3.79 (t, J=6.4 Hz, 2H), 6.38 (d, J=8.8 Hz, 2H), 6.61-6.58 (m, 5H), 6.71 (d, J=8.4 Hz, 2H), 6.93 (d, J=8.4 Hz, 2H), 7.02 (t, J=8.0 Hz, 1H), 9.12 (s, 1H), 9.36 (s, 1H), 12.11 (s, 1H). LCMS (ESI): m/z 431 (M−H)⁻.

### Example 15 (35)

### 2-{[2-({3-[2,2-Bis(4-hydroxyphenyl)-1-propylethenyl]phenyl}oxy)ethyl]oxy}ethyl acetate (35)

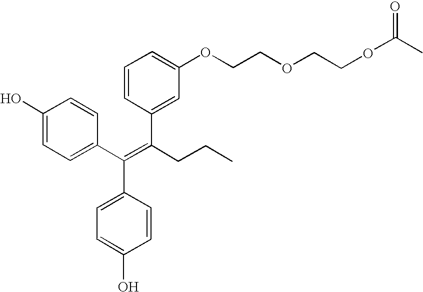

### Step 1: 1-[3-({2-[(2-Hydroxyethyl)oxy]ethyl}oxy)phenyl]-1-butanone (33)

The general O-alkylation procedure described for 13 was employed using 1-(3-hydroxyphenyl)-1-butanone 25 (1.0 g, 6.1 mmol), 2-[(2-chloroethyl)oxy]ethanol (2.0 mL, 18.3 mmol), K₂CO₃ (1.68 g, 12.2 mmol), and acetone under N₂. The reaction mixture was refluxed for 48 h. Regular work-up followed by purification gave the title product 33 (contains some amount of 2-[(2-chloroethyl)oxy]ethanol), which was used as such in the next step. LCMS (ESI): m/z 253 (M+H)⁺.

### Step 2: 2-({2-[(3-Butanoylphenyl)oxy]ethyl}oxy)ethyl acetate (34)

To a stirred solution of 33 in CH₂Cl₂ (150 mL) were added Et₃N (2.0 mL), DMAP (0.150 mg), and acetic anhydride (2.5 mL) sequentially at 5° C. under N2. The resultant reaction mixture was stirred at RT for 2 h. The reaction mixture was washed with H₂O (1×40 mL), brine (1×40 mL), dried (Na₂SO₄), and then filtered. The filtrate was concentrated under reduced pressure to afford the crude product which was purified by flash SiO₂ column chromatography to afford 1.49 g (83%) of the title product 34 as a colorless oil. ¹H NMR (400 MHz, DMSO-d₆): δ 0.90 (t, J=7.6 Hz, 3H), 1.60 (app. sextet, J=7.2 Hz, 2H), 1.98 (s, 3H), 2.97 (d, J=7.2 Hz, 2H), 3.65 (t, J=4.8 Hz, 2H), 3.75 (t, J=4.4 Hz, 2H), 4.11 (t, J=4.4 Hz, 2H), 4.14 (t, J=4.4 Hz, 2H), 7.20 and 7.18 (dd, J₁=8.0 Hz, J₂=2.4 Hz, 1H), 7.40 (app. t, J=8.0 Hz, 1H), 7.43 (br s, 1H), 7.53 (d, J=8.0 Hz, 1H). LCMS (ESI): m/z 295 (M+H)⁺.

### Step 3: 2-{[2-({3-[2,2-Bis(4-hydroxyphenyl)-1-propylethenyl]phenyl}oxy)ethyl]oxy}ethyl acetate (35)

The general McMurry procedure described for 1 was employed using bis(4-hydroxyphenyl)methanone (0.352 g, 1.64 mmol), 1-[3-({2-[(2-Hydroxyethyl)oxy]ethyl}oxy)phenyl]-1-butanone 34, TiCl₄.2THF (3.29 g, 9.82 mmol), Zn (1.07 g, 16.42 mmol), and THF. Regular work-up followed by purification gave 0.746 g (95%) of the title product 35 as a white foam. ¹H NMR (400 MHz, DMSO-d₆): δ 0.73 (t, J=7.2 Hz, 3H), 1.21 (app. sextet, J=7.6 Hz, 2H), 1.99 (s, 3H), 2.30 (t, J=8.0 Hz, 2H), 3.60 (t, J=4.4 Hz, 2H), 3.63 (t, J=4.8 Hz, 2H), 3.91 (t, J=4.8 Hz, 2H), 4.09 (t, J=4.8 Hz, 2H), 6.39 (d, J=8.8 Hz, 2H), 6.64-6.59 (m, 5H), 6.72 (d, J=8.4 Hz, 2H), 6.94 (d, J=8.4 Hz, 2H), 7.04 (t, J=8.0 Hz, 1H), 9.12 (s, 1H), 9.36 (s, 1H). LCMS (ESI): m/z 477 (M+H)⁺.

### Example 16 (36)

### 4,4′-{2-[3-({2-[(2-Hydroxyethyl)oxy]ethyl}oxy)phenyl]-1-pentene-1,1-diyl}diphenol (36)

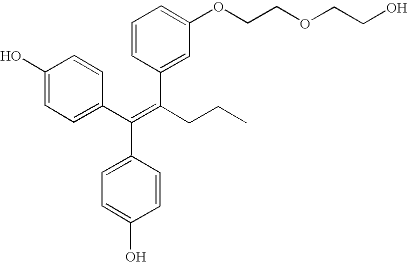

### 4,4′-{2-[3-({2-[(2-hydroxyethyl)oxy]ethyl}oxy)phenyl]-1-pentene-1,1-diyl}diphenol (36)

To a stirred solution of 35 in MeOH (50 mL) was added K₂CO₃ (0.985 g, 7.14 mmol) at RT. The reaction mixture was stirred at RT for 1 h. The reaction mixture was filtered through a pad of celite using MeOH as a solvent. The filtrate was concentrated under reduced pressure to afford the crude product. The product was purified by flash SiO₂ column chromatography using CHCl₃: MeOH (100:00 to 9:1) as an eluent to afford 0.588 g (95%) the title product 36 as an off-white solid. ¹H NMR (400 MHz, DMSO-d₆): δ 0.73 (t, J=7.2 Hz, 3H), 1.21 (app. sextet, J=7.6 Hz, 2H), 2.30 (t, J=8.0 Hz, 2H), 3.43 (t, J=4.8 Hz, 2H), 3.47 (t, J=5.6 Hz, 2H), 3.62 (t, J=4.4 Hz, 2H), 3.90 (t, J=5.2 Hz, 2H), 4.60 (t, J=5.6 Hz, 1H), 6.39 (d, J=8.8 Hz, 2H), 6.63-6.59 (m, 5H), 6.93 (d, J=8.4 Hz, 2H), 7.04 (d, J=7.6 Hz, 2H), 9.13 (s, 1H), 9.3.6 (s, 1H). LCMS (ESI): m/z 435 (M+H)⁺.

### Example 17 (40)

### (2E)-3-{3-[1-Ethyl-2,2-bis(4-fluorophenyl)ethenyl]phenyl}-2-propenoic (40)

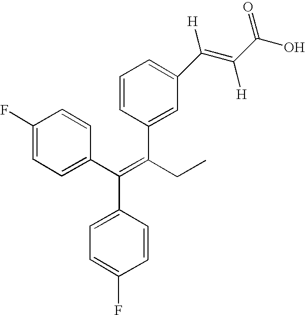

### Step 1: Ethyl(2E)-3-(3-propanoylphenyl)-2-propenoate (37)

To a round bottom flask were added 1-(3-bromophenyl)-1-propanone (4.26 g, 0.02 mol), ethyl acrylate (11 mL, 0.10 mol), dichlorobis(triphenylphosphine) palladium (II) (1.4 g, 0.002 mol, 10 mol %), triethylamine (11 mL, 0.08 mol), and DMF (25 mL). The stirred reaction mixture was heated to 100° C. for 12 h under a nitrogen atmosphere. Standard work-up followed by purification gave 3.675 g (79%) of the title compound 37 as a pale yellow solid. ¹H NMR (400 MHz, CDCl₃): δ 1.21 (t, J=7.2 Hz, 3H), 1.32 (t, J=7.2 Hz, 3H), 3.00 (q, J=7.2 Hz, 2H), 4.25 (q, J=7.2 Hz, 2H), 6.49 (d, J=16.0 Hz, 1H), 7.46 (app. t, J=8.0 Hz, 1H), 7.67 (br d, J=8.0 Hz, 1H), 7.69 (d, J=16.4 Hz, 1H), 7.93 (br d, J=7.6 Hz, 1H), 8.08 (br s, 1H).

### Step 2: Ethyl(2E)-3-[3-(2,2-dibromo-1-ethylethenyl)phenyl]-2-propenoate (38)

To a stirred solution of ethyl(2E)-3-(3-propanoylphenyl)-2-propenoate 37 (3.25 g, 14 mmol) in CH₂Cl₂ were added PPh₃ (14.69 g, 56 mmol) and CBr₄ (9.29 g, 28 mmol) at room temperature. The resultant reaction mixture was refluxed for 8 h then cooled to RT and diluted with hexanes (500 mL). The hexane layer was separated via filtertion and the filtrate was concentrated under reduced pressure. The crude reaction mixture was purified by silica gel column chromatography to afford 1.1 g (20%) of the title product 38 as an oil. ¹H NMR (400 MHz, CDCl₃): δ 0.97 (t, J=8.0 Hz, 3H), 1.33 (t, J=6.8 Hz, 3H), 2.59 (q, J=7.2 Hz, 2H), 4.26 (q, J=6.8 Hz, 2H), 6.44 (d, J=16.0 Hz, 1H), 7.18 (br d, J=7.6 Hz, 1H), 7.33 (br s, 1H), 7.39 (app. t, J=8.0 Hz, 1H), 7.47 (br d, J=7.6 Hz, 1H), 6-77 (d, J=16.0, 1H).

### Step 3: (2E)-3-[3-(2,2-Dibromo-1-ethylethenyl)phenyl]-2-propenoic acid (39)

The general saponification procedure described for 3 was employed using ethyl(2E)-3-[3-(2,2-dibromo-1-ethylethenyl)phenyl]-2-propenoate 38 (1.00 g, 2.58 mmol). Standard work-up followed by purification gave 0.928 g (˜100%) of the title compound 39 as a white solid. ¹H NMR (400 MHz, DMSO-d₆): δ 0.87 (t, J=7.2 Hz, 3H), 2.56 (q, J=7.6 Hz, 2H), 6.58 (d, J=16.0 Hz, 1H), 7.24 (br d, J=8.0 Hz, 1H), 7.43 (app. t, J=7.6 Hz, 1H), 7.55 (br s, 1H), 7.58 (d, J=16.4 Hz, 1H), 7.65 (br d, J=8.0 Hz, 1H). LCMS (ESI): m/z 357 (M−H)⁻ and 361 (M−H)⁻.

### Step 4: (2E)-3-{3-[1-Ethyl-2,2-bis(4-fluorophenyl)ethenyl]phenyl}-2-propenoic (40)

The general Suzuki procedure described for 7 was employed using (2E)-3-[3-(2,2-dibromo-1-ethylethenyl)phenyl]-2-propenoic acid 39 (120 mg, 0.33 mmol) and (4-fluorophenyl)boronic acid (231 mg, 1.65 mmol). Standard work-up followed by purification gave the title compound 40 as a white solid. LCMS (APCI): m/z 391 (M+H)⁺ and 389 (M−H)⁻.

### Example 18 (41)

### (2E)-3-{3-[2,2-Bis(4-Cyanophenyl)-1-ethylethenyl]phenyl}-2-propenoic acid (41)

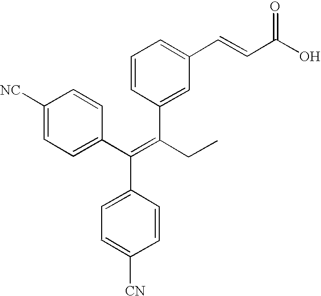

### (2E)-3-{3-[2,2-Bis(4-Cyanophenyl)-1-ethylethenyl]phenyl}-2-propenoic acid (41)

The general Suzuki procedure described for 7 was employed using (2E)-3-[3-(2,2-dibromo-1-ethylethenyl)phenyl]-2-propenoic acid 39 (120 mg, 0.33 mmol) and (4-fluorophenyl)boronic acid (0.231 g, 1.65 mmol). Standard work-up followed by purification gave the title compound 41 as a white solid. LCMS (APCI): m/z 403.05 (M−H)⁻.

### Example 19 (42)

### (2E)-3-{3-[2,2-Bis(4-Cyano-3-fluorophenyl)-1-ethylethenyl]phenyl}-2-propenoic acid (42)

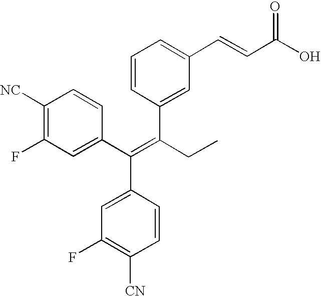

### (2E)-3-{3-[2,2-Bis(4-Cyano-3-fluorophenyl)-1-ethylethenyl]phenyl}-2-propenoic acid (42)

The general Suzuki procedure described for 7 was employed using 2E)-3-[3-(2,2-dibromo-1-ethylethenyl)phenyl]-2-propenoic acid 39 (120 mg, 0.33 mmol) and (4-fluorophenyl)boronic acid (231 mg, 1.65 mmol). Standard work-up followed by purification gave the title compound 42 as a white solid. ¹H NMR (400 MHz, DMSO-d₆): δ 0.84 (t, J=7.2 Hz, 3H), 2.39 (d, J=7.2 Hz, 2H), 6.43 (d, J=15.6 Hz, 1H), 6.96 and 6.94 (dd, J₁=8.0 Hz, J₂=1.2 Hz, 1H), 7.06 (d, J=10.8 Hz, 1H), 7.13 (d, J=8.0 Hz, 1H), 7.26 (t, J=8.0 Hz, 1H), 7.35 and 7.33 (dd, J₁=8.0 Hz, J₂=0.8 Hz, 1H), 7.48 (d, J=16.0 Hz, 1H), 7.50 (d, J=14.8 Hz, 1H), 7.51 (d, J=12.0 Hz, 1H), 7.65 (d, J=7.6 Hz, 1H), 7.99 (t, J=7.2 Hz, 1H), 12.41 (s, 1H). LCMS (APCI): m/z 463 (M+Na)⁺ and 439 (M−H)⁻.

### Example 20 (46)

### 4,4′-{2-[3-({2-[(2-hydroxyethyl)oxy]ethyl}oxy)phenyl]-1-hexene-1,1-diyl}diphenol (46)

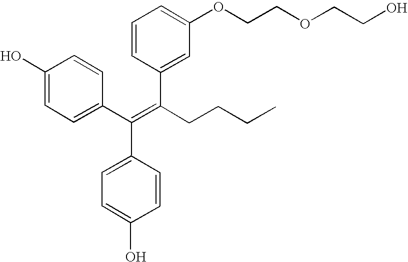

### Step 1: 1-[3-(Methyloxy)phenyl]-1-pentanone (43)

To a stirred solution of N-methyl-N,3-bis(methyloxy)benzamide 11 (7.0 g, 0.036 mol) in THF was added 2 M solution of n-butyl magnesium chloride (36 mL, 0.072 mol) slowly at RT. The resultant reaction mixture was allowed to stir at room temperature for 0.5 h then poured into 20% aqueous HCl (300 mL) and extracted with EtOAc (3×100 mL). The combined organic layer was washed with brine (1×100 mL), dried, filtered, and concentrated under reduced pressure to afford 6.615 g (96%) of the title compound 43. ¹H NMR (400 MHz, CDCl₃): δ 94 (t, J=7.2 Hz, 3H), 1.39 (app. sextet, J=7.6 Hz, 2H), 1.71 (app. quintet, J=7.6 Hz, 2H), 2.94 (t, J=7.6 Hz, 2H), 3.85 (s, 3H), 7.10 and 7.07 (dd, J₁=8.0 Hz, J₂=2.4 Hz, 1H), 7.35 (t, J=8.0 Hz, 1H), 7.48 (app. t, J=4.0 Hz, 1H), 7.53 (br d, J=8.0 Hz, 1H).

### Step 2: 1-(3-Hydroxyphenyl)-1-pentanone (44)

The general demethylation procedure described for 13 was employed with 1-[3-(methyloxy)phenyl]-1-pentanone 43 (6.0 g, 0.031 mol). Standard work-up followed by purification gave 5.30 g (95%) of the title compound 44 as an off-white solid. ¹H NMR (400 MHz, CDCl₃): δ 0.93 (t, J=7.2 Hz, 3H), 1.39 (app. sextet, J=7.2 Hz, 2H), 1.71 (app. quintet, J=7.6 Hz, 2H), 2.96 (t, J=7.2 Hz, 2H), 7.11 and 7.09 (dd, J₁=8.0 Hz, J₂=2.4 Hz, 1H), 7.32 (app. t, J=7.6 Hz, 1H), 7.51 (br d, J=8.0 Hz, 7.60 (br t, J=2.0 Hz, 1H).

### Step 3: 1-[3-({2-[(2-Hydroxyethyl)oxy]ethyl}oxy)phenyl]-1-pentanone (45)

A round bottom flask was charged with 1-(3-hydroxyphenyl)-1-pentanone 44 (1.15 g, 6.45 mmol), K₂CO₃ (2.68 g, 19.4 mmol), acetone (40 mL), and 2-[(2-chloroethyl)oxy]ethanol (1.7 mL, 16.13 mmol) under N₂. The reaction mixture was refluxed for 60 h and cooled to room temperature. The reaction mixture was filtered and the filtrate concentrated under reduced pressure. The crude product was purified by SiO₂ column chromatography using hexanes and EtOAc (19:1 to 1:1) as an eluent to give 1.655 g (96%) of the title compound 45 as a solid. ¹H NMR (400 MHz, CDCl₃): δ 0.92 (t, J=7.2 Hz, 3H), 1.38 (app. sextet, J=7.2 Hz, 2H), 1.68 (app. quintet, J=7.6 Hz, 2H), 2.47 (t, J=6.4 Hz, 1H), 2.91 (t, J=7.6 Hz, 2H), 3.65 (t, J=4.0 Hz, 2H), 3.74 (app. q, J=4.0 Hz, 2H), 3.85 (t, J=4.4 Hz, 2H), 4.17 (t, J=4.8 Hz, 2H), 7.10 and 7.08 (dd, J₁=6.0 Hz, J₂=2.4 Hz, 1H), 7.33 (app. t, J=8.0 Hz, 1H), 7.49 (br d, J=2.4 Hz, 1H), 7.52 (br d, J=7.6 Hz, 1H). LCMS (ESI): m/z 267 (M+H)⁺.

### Step 4: 4,4′-{2-[3-({2-[(2-Hydroxyethyl)oxy]ethyl}oxy)phenyl]-1-hexene-1,1-diyl}diphenol (46)

The general McMurry procedure described for 1 (Example 1, Step 1) was employed using bis(4-hydroxyphenyl)methanone (0.143 g, 0.666 mmol) and 1-[3-({2-[(2-hydroxyethyl)oxy]ethyl}oxy)phenyl]-1-pentanone 45 (0.532 g, 2.0 mmol). Standard work-up followed by purification gave 0.275 g (92%) of the title compound 46 as an off-white foam. ¹H NMR (400 MHz, DMSO-d₆): δ 0.71 (t, J=6.8 Hz, 3H), 1.16 (m, 4H), 2.33 (t, J=8.0 Hz, 2H), 3.42 (t, J=4.8 Hz, 2H), 3.47 (t, J=4.8 Hz, 2H), 3.62 (t, J=4.0 Hz, 2H), 3.90 (t, J=4.0 Hz, 2H), 4.20 (br s, 1H), 6.38 (d, J=8.8 Hz, 2H), 6.59 (d, J=8.8 Hz, 2H), 6.64-6.61 (br d, J=12.0 Hz, 3H), 6.70 (d, J=8.4 Hz, 2H), 6.93 (d, J=8.4 Hz, 2H), 7.04 (d, J=8.0 Hz, 1H), 9.13 (s, 1H), 9.37 (s, 1H). LCMS (ESI): m/z 449 (M+H)⁺.

### Example 21 (47)

### 2-{([2-({3-[(1-Butyl-2,2-bis(4-fluorophenyl)ethenyl]phenyl}oxy)ethyl]oxy}ethanol (47)

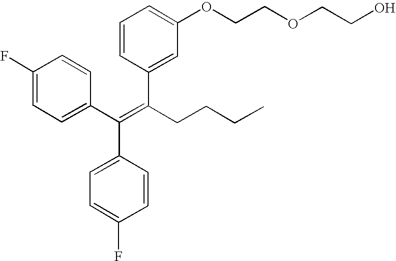

### 2-{[2-({3-[1-Butyl-2,2-bis(4-fluorophenyl)ethenyl]phenyl}oxy)ethyl]oxy}ethanol (47)

The general McMurry procedure described for 1 was employed using bis(4-fluorophenyl)methanone (0.150 g, 0.69 mmol) and 1-[3-({2-[(2-hydroxyethyl)oxy]ethyl}oxy)phenyl]-1-pentanone 45 (0.550 g, 2.07 mmol). Standard work-up followed by purification gave 0.165 g (77%) of the title compound 47 as a white foam. ¹H NMR (400 MHz, DMSO-d₆): δ 0.71 (t, J=6.8 Hz, 3H), 1.21-1.12 (m, 4H), 2.31 (t, J=7.6 Hz, 2H), 3.43 (t, J=4.8 Hz, 2H), 3.47 (t, J=4.8 Hz, 2H), 3.62 (t, J=4.0 Hz, 2H), 3.92 (t, J=4.0 Hz, 2H), 4.60 (t, J=4.8 Hz, 1H), 6.69-6.63 (m, J=6.0 Hz, 3H), 6.88 (d, J=7.2 Hz, 4H), 7.07 (app. t, J=7.6 Hz, 1H), 7.22-7.17 (m, J=2.8 Hz, 4H). LCMS (APCI): m/z 453 (M+H)⁺.

### Example 22 (49)

### 4,4′-(2-{3-[(2-Hydroxyethyl)oxy]phenyl}-1-hexene-1,1-diyl)diphenol (49)

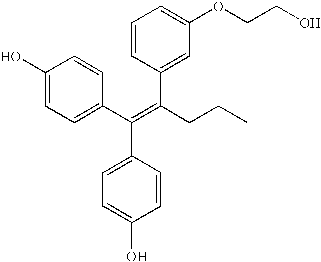

### Step 1: Ethyl [(3-pentanoylphenyl)oxy]acetate (48)

A round bottom flask was charged with 1-(3-hydroxyphenyl)-1-pentanone 44 (1.33 g, 7.46 mmol), K₂CO₃ (2.06 g, 14.92 mmol), acetone (50 mL), and ethyl bromoacetate (1.65 mL, 14.92 mmol) under N₂. The reaction mixture was refluxed for 18 h and cooled to room temperature. Reaction mixture was filtered and the filtrate concentrated under reduced pressure. The crude product was purified by SiO₂ column chromatography using hexanes and EtOAc (19:1 to 1:1) as an eluent to give 1.510 g (77%) of the title compound 48 as an oil. ¹H NMR (400 MHz, CDCl₃): δ 0.93 (t, J=7.2 Hz, 3H), 1.29 (t, J=7.2 Hz, 3H), 1.38 (app. sextet, 2H), 1.69 (app. quintet, J=7.2 Hz, 2H), 2.92 (t, J=7.2 Hz, 2H), 4.26 (q, J=7.2 Hz, 2H), 4.66 (s, 2H), 7.12 and 7.10 (dd, J₁=8.0 Hz, J₂=2.8 Hz, 1H), 7.36 (app. t, J=7.6 Hz, 1H), 7.47 (app, t, J=1.6 Hz, 7.57 (d, J=7.6 Hz, 1H). LCMS (APCI): m/z 265 (M+H)⁺.

### Step 2: Ethyl({3-[1-butyl-2,2-bis(4-hydroxyphenyl)ethenyl]phenyl}oxy)acetate (49)

The general McMurry procedure, described for 1 (Example 1, Step 1) was employed using bis(4-hydroxyphenyl)methanone (0.364 g, 1.70 mmol) and ethyl [(3-pentanoylphenyl)oxy]acetate 48 (1.350 g, 5.11 mmol). Standard work-up followed by purification gave 0.310 g (41%) of the title compound 49 as off-white foam. ¹H NMR (400 MHz, DMSO-d₆): δ 0.71 (t, J=6.8 Hz, 3H), 1.20-1.12 (m, 7H), 2.32 (t, J=8.0 Hz, 2H), 4.12 (q, J=6.8 Hz, 2H), 4.59 (S, 2H), 6.38 (d, J=8.4 Hz, 2H), 6.58 (d, J=8.4 Hz, 2H), 6.64-6.61 (m, 3H), 6.71 (d, J=8.4 Hz, 2H), 6.93 (d, J=8.4 Hz, 2H), 7.06-7.02 (m, 1H), 9.13 (s, 1H), 9.37 (s, 1H). LCMS (APCI): m/z 445 (M−H)⁻.

### Step 3: 4,4′-(2-{3-[(2-Hydroxyethyl)oxy]phenyl}-1-hexene-1,1-diyl)diphenol (50)

To a stirred solution of ethyl({3-[1-butyl-2,2-bis(4-hydroxyphenyl)ethenyl]phenyl}oxy)acetate 49 (0.130 mg, 0.336 mmol) in THF (25 mL) was added 1 M solution of LiAlH₄ (1.0 mL, 1.0 mmol) under N₂ atmosphere at room temperature and stirred for 0.5 h. Standard work-up and purification gave 0.130 (96%) of the title compound 50 as a white solid. ¹H NMR (400 MHz, DMSO-d₆): δ 0.71 (t, J=7.2 Hz, 3H), 1.21-1.11 (m, 4H), 2.33 (br d, J=8.4 Hz, 2H), 3.62 (q, J=4.8 Hz, 2H), 3.79 (t, J=5.2 Hz, 2H), 4.79 (t, J=5.6 Hz, 1H), 6.38 (d, J=8.4 Hz, 2H), 6.63-6.59 (m, 5H), 8.4 (d, J=8.8 Hz, 2H), 6.93 (d, J=8.4 Hz, 2H), 7.03 (t, J=7.6 Hz, 1H), 9.12 (s, 1H), 9.36 (s, 1H). LCMS (ESI): m/z 405 (M+H)⁺.

### Example 23 (51)

### 3-[1-Ethyl-2,2-bis(4-hydroxyphenyl)ethenyl]benzoic acid (51)

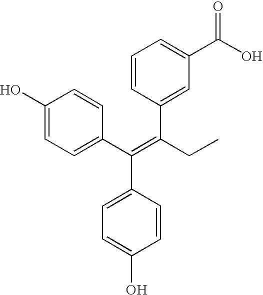

### 3-[1-Ethyl-2,2-bis(4-hydroxyphenyl)ethenyl]benzoic acid (51)

The general McMurry procedure described for 1 was employed using bis(4-hydroxyphenyl)methanone (0.400 g, 1.87 mmol) and 3-propanoylbenzoic acid (1.00 g, 5.6 mmol). Standard work-up followed by purification gave the title compound 51 as an off-white solid. ¹H NMR (400 MHz, DMSO-d₆): δ 0.82 (t, J=7.2 Hz, 3H), 2.39 (q, J=7.6 Hz, 2H), 6.38 (d, J=8.4 Hz, 2H), 6.55 (d, J=8.4 Hz, 2H), 6.74 (d, J=8.4 Hz, 3H), 6.95 (d, J=8.4 Hz, 2H), 7.18 (br d, J=5.6 Hz, 2H), 7.68 (br s, 2H), 9.32 (br s, 1H), 9.52 (br s, 1H). LCMS (ESI): m/z 359 (M−H)⁻.

### Example 24 (55)

### 4,4′-{2-[3-[(2-Hydroxyethyl)oxy]-4-(methyloxy)phenyl]-1-butene-1,1-diyl}diphenol (55)

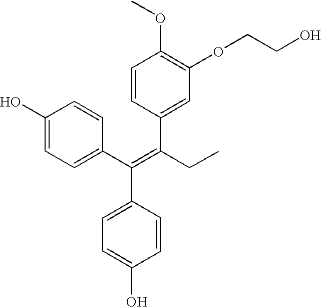

### Step 1: 1-[3-Hydroxy-4-(methyloxy)phenyl]-1-propanone (52)

The general O-alkylation procedure described for 14 (Example 7, Step 4) was employed using 1-(3,4-dihydroxyphenyl)-1-propanone (5.00 g, 30 mmol), K₂CO₃ (8.28 g, 60 mmol), methyl iodide (2.05 mL, 33 mmol), and acetone (150 mL). Standard work-up followed by purification gave 2.71 g (50%) of the title product 52 as a white solid. ¹H NMR (400 MHz, CDCl₃): δ 1.20 (t, J=7.2 Hz, 3H), 2.93 (q, J=7.2 Hz, 2H), 3.95 (s, 3H), 5.65 (s, 1H), 6.88 (d, J=8.0 Hz, 1H), 7.54 (s, 1H), 7.56 and 7.54 (app.dd, J₁=10.0 Hz, J₂=2.0 Hz, 1H). LCMS (ESI): m/z 181 (M+H)⁺.

### Step 2: Ethyl {[2-(methyloxy)-5-propanoylphenyl]oxy}acetate (53)

The general O-alkylation procedure described for 14 (Example 7, Step 4) was employed using 1-[3-hydroxy-4-(methyloxy)phenyl]-1-propanone 52 (1.25 g, 6.94 mmol), bromoethylacetate (2.0 mL, 17.35 mmol), K₂CO₃ (1.92 g, 13.88 mmol), and acetone (75 mL). Standard work-up followed by flash column chromatography gave 1.76 g (95%) of the title product 53 as a white solid. ¹H NMR (400 MHz, CDCl₃): δ 0.80 1.19 (t, J=7.2 Hz, 3H), 1.29 (t, J=7.2 Hz, 3H), 2.93 (q, J=6.8 Hz, 2H), 3.94 (s, 3H), 4.26 (q, J=7.2 Hz, 2H), 4.73 (s, 2H), 6.91 (d, J=8.4 Hz, 1H), 7.47 (d, J=2.0 Hz, 1H), 7.62 and 7.64 (dd, J₁=8.4 Hz, J₂=2.0 Hz, 1H). LCMS (ESI): m/z 267 (M+H)⁺.

### Step 3: Ethyl {[5-[1-Ethyl-2,2-bis(4-hydroxyphenyl)ethenyl]-2-(methyloxy)phenyl]oxy}acetate (54)

The standard McMurry procedure described for 1 (Example 1, Step 1) was employed using bis(4-hydroxyphenyl)methanone (0.416 g, 1.94 mmol) and ethyl {[2-(methyloxy)-5-propanoylphenyl]oxy}acetate 53 (1.550 g, 5.82 mmol). Standard work-up followed by flash column chromatography gave 0.655 g (75%) of the title 54 compound as an off-white foam. ¹H NMR (400 MHz, DMSO-d₆): δ 0.81 (t, J=7.6 Hz, 3H), 1.17 (t, J=7.2 Hz, 3H), 2.33 (q, J=7.2 Hz, 2H), 3.69 (s, 3H), 4.11 (q, J=6.8 Hz, 2H), 4.47 (s, 2H), 6.40 (d, J=8.4 Hz, 2H), 6.55 (d, J=1.6 Hz, 1H), 6.57 (d, J=8.8 Hz, 2H), 6.62 and 6.60 (dd, J₁=8.4 Hz, J₂=2.0 Hz, 1H), 6.70 (d, J=8.4 Hz, 2H), 6.76 (d, J=8.4 Hz, 1H), 6.91 (d, J=8.4 Hz, 2H), 9.13 (s, 1H), 9.36 (s, 1H). LCMS (ESI): m/z 449 (M+H)⁺.

### Step 4: 4,4′-{2-[3-[(2-Hydroxyethyl)oxy]-4-(methyloxy)phenyl]-1-butene-1,1-diyl}diphenol (55)

Standard LAH reduction described for 16 (Example 7, Step 6) using ethyl {[5-[1-ethyl-2,2-bis(4-hydroxyphenyl)ethenyl]-2-(methyloxy)phenyl]oxy}acetate 54 (0.460 g, 1.026 mmol) and a 1 M solution of LAH (5.13 mL, 5.13 mmol). Standard work-up followed by purification gave 0.395 g (95%) of the title compound 55 as a white solid. ¹H NMR (400 MHz, DMSO-d₆): δ 0.83 (t, J=7.6 Hz, 3H), 2.35 (q, J=6.8 Hz, 2H), 3.58 (q, J=4.8 Hz, 2H), 3.67 (s, 3H), 3.68 (app. t, J=5.2 Hz, 2H), 4.76 (t, J=5.6 Hz, 1H), 6.40 (d, J=8.4 Hz, 2H), 6.59 (app. d, J=8.4 Hz, 2H), 6.60 (br s, 2H), 6.70 (d, J=8.4 Hz, 2H), 7.34 (d, J=8.8 Hz, 1H), 6.93 (d, J=8.4 Hz, 2H), 9.12 (s, 1H), 9.35 (s, 1H). LCMS (ESI): m/z 407 (M+H)⁺.

### Example 25 (57)

### 4,4′-{2-[3-({2-[(2-Hydroxyethyl)oxy]ethyl}oxy)-4-(methyloxy)phenyl]-1-butene-1,1-diyl}diphenol (57)

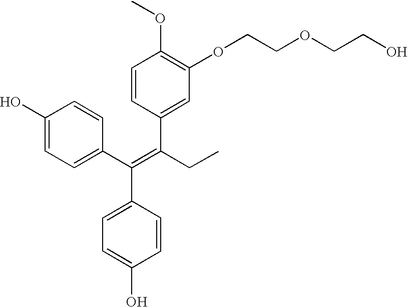

### Step 1: 1-[3-({2-[(2-Hydroxyethyl)oxy]ethyl}oxy)-4-(methyloxy)phenyl]-1-propanone (56)

The general O-alkylation procedure described for 14 (Example 7, Step 4) was employed using 1-[3-hydroxy-4-(methyloxy)phenyl]-1-propanone 51 (0.800 g, 4.44 mmol), K₂CO₃ (1.23 g, 8.88 mmol), 2-[(2-chloroethyl)oxy]ethanol (1.41 mL, 13.32 mmol), and acetone (100 mL). Standard work-up followed by purification gave the title product 56 (contains some amount of 2-[(2-chloroethyl)oxy]ethanol) which was used as such in the next step. LCMS (ESI): m/z 269 (M+H)⁺.

### Step 2: 4,4′-{2-[3-({2-[(2-Hydroxyethyl)oxy]ethyl}oxy)-4-(methyloxy)phenyl]-1-butene-1,1-diyl}diphenol (57)

The general McMurry procedure described for 1 (Example 1, Step 1) was employed using bis(4-hydroxyphenyl)methanone (0.300 g, 1.40 mmol), 1-[3-({2-[(2-hydroxyethyl)oxy]ethyl}oxy)-4-(methyloxy)phenyl]-1-propanone 56 (˜1.5 g, from step 1). Regular work-up and purification gave 0.586 g (93%) of the title product 57 as a white foam. ¹H NMR (400 MHz, DMSO-d₆): δ 0.83 (t, J=7.6 Hz, 3H), 2.36 (q, J=7.2 Hz, 2H), 3.41 (t, J=5.2 Hz, 2H), 3.47 (br s, 2H), 3.56 (d, J=4.40 Hz, 2H), 3.67 (s, 3H), 3.79 (d, J=4.8 Hz, 2H), 4.58 (br s, 1H), 6.40 (d, J=8.0 Hz, 2H), 6.63-6.58 (m, 4H), 6.70 (d, J=8.8 Hz, 2H), 6.75 (d, J=8.4 Hz, 1H), 6.92 (d, J=8.4 Hz, 2H), 9.12 (s, 1H), 9.35 (s, 1H). LCMS (ESI): m/z 451 (M+H)⁺.

### Example 26 (58)

### 3-[1-Ethyl-2,2-bis(4-hydroxyphenyl)ethenyl]benzonitrile (58)

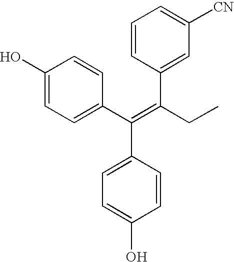

A 10 mL glass tube was charged with 4,4′-[2-(3-Bromophenyl)-1-butene-1,1-diyl]diphenol (1) (791 mg, 2 mmol), CuCN (448 mg, 5 mmol), and NMP (3 mL) under N₂The sealed tube was heated to 180° C. and stirred for 15 h. The mixture was cooled to RT, removed the seal, and poured into H₂O (50 mL) and the mixture was extracted with EtOAc (4×50 mL). The combined organic layer was washed with brine (1×15 mL), dried over Na₂SO₄, filtered and the filtrate was concentrated under reduced pressure. The crude product was purified by flash SiO₂ column chromatography to afford 586 mg (86%) of the title compound 58 as an off-white solid. ¹H NMR (400 MHz, DMSO-d₆): δ 0.82 (t, J=7.6 Hz, 3H), 2.42 (q, J=7.6 Hz, 2H), 6.42 (d, J=8.8 Hz, 2H), 6.58 (d, J=8.8 Hz, 2H), 6.73 (d, J=8.4 Hz, 2H), 7.96 (d, J=8.4 Hz, 2H), 7.38-7.32 (m, 2H), 7.50 (br. s, 1H), 7.54 and 7.52 (m, 1H), 9.23 (s, 1H), 9.44 (s, 1H). LCMS (ESI): m/z 359 (M+H₂O).

### Example 27 (59)

### 5-[1-Ethyl-2,2-bis(4-hydroxyphenyl)ethenyl]-2-fluorobenzonitrile (59)

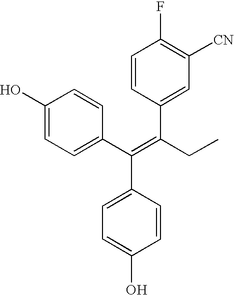

The procedure described for 58 (Example 26) was employed using 4,4′-[2-(3-Bromo-4-fluorophenyl)-1-butene-1,1-diyl]diphenol (1) (0.827 g, 2 mmol), CuCN (0.448 g, 5 mmol), and NMP (3 mL). Standard work-up followed by purification gave 0.618 g (86%) of the title product 59 as an off-white solid. ¹H NMR (400 MHz, DMSO-d₆): δ 0.83 (t, J=7.2 Hz, 3H), 2.41 (q, J=7.2 Hz, 2H), 6.44 (d, J=8.8 Hz, 2H), 6.59 (d, J=8.8 Hz, 2H), 6.73 (d, J=8.4 Hz, 2H), 7.29 (t, J=8.8 Hz, 2H), 7.41-7.37 (m, 1H), 7.63 and 7.61 (dd, J₁=6.4 Hz, J₂=2 Hz, 1H), 7.60 (d, J=8.0 Hz, 2H), 9.25 (s, 1H), 9.44 (s, 1H). LCMS (ESI): m/z 368 (M−H)⁻.

### Prophetic Examples

The following examples may be prepared by methods analogous to those herein described:

### Example 28 (#)

### {[5-[1-Ethyl-2,2-bis(4-hydroxyphenyl)ethenyl]-2-(methyloxy)phenyl]oxy}acetic acid

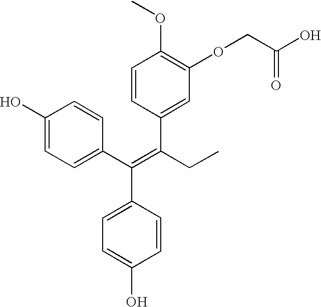

### Example 29 (#)

### 4-{[5-[1-Ethyl-2,2-bis(4-hydroxyphenyl)ethenyl]-2-(methyloxy)phenyl]oxy}butanoic acid

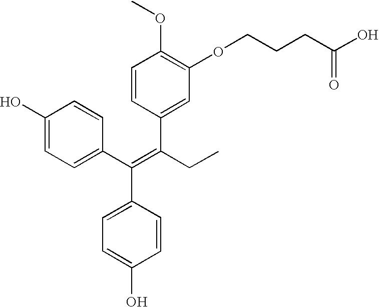

### Example 30(#)

### (2E)-3-[5-[1-Ethyl-2,2-bis(4-hydroxyphenyl)ethenyl]-2-(methyloxy)phenyl]-2-propenoic acid

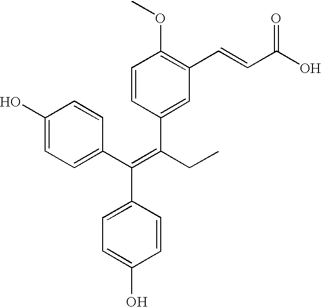

### Example 31 (#)

### (2E)-3-{3-[2,2-Bis(4-Hydroxyphenyl)-1-propylethenyl]phenyl}-2-propenoic acid

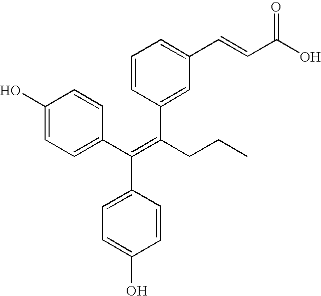

### Example 32 (#)

### (2E)-3-{3-[1-Butyl-2,2-bis(4-hydroxyphenyl)ethenyl]phenyl}-2-propenoic acid

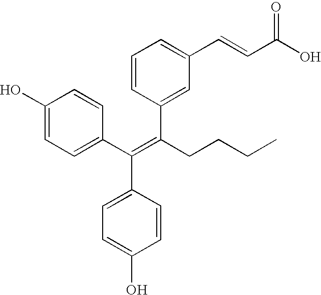

### Example 33 (#)

### 4,4′-{2-[3-(Methylsulfonyl)phenyl]-1-butene-1,1-diyl}diphenol

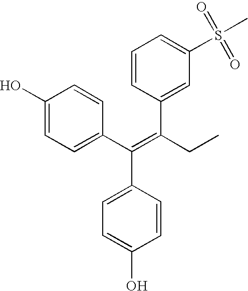

### Example 34 (#)

### 4,4′-[2-[4-Fluoro-3-(methylsulfonyl)phenyl]-1-butene-1,1-diyl]diphenol

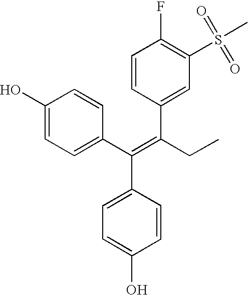

### Example 35 (#)

### 2-({2-[(3-{1-Ethyl-2,2-bis[4-(methyloxy)phenyl]ethenyl}phenyl)oxy]ethyl}oxy)ethanol

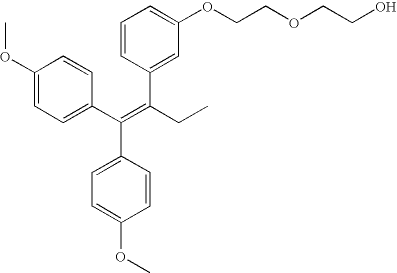

### Example 36 (#)

### 2-({2-[(3-{1-Ethyl-2,2-bis[4-(methylthio)phenyl]ethenyl}phenyl)oxy]ethyl}oxy)ethanol

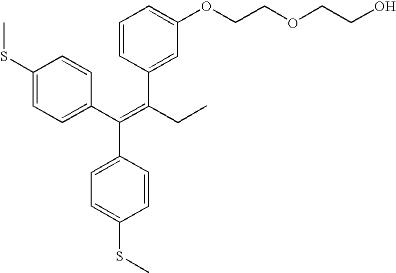

### Example 37 (#)

### 2-({2-[(3-{1-Ethyl-2,2-bis[4-(methylsulfinyl)phenyl]ethenyl}phenyl)oxy]ethyl}oxy)ethanol

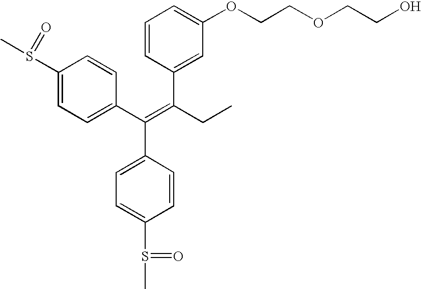

### Example 38 (#)

### 2-({2-[(3-{1-Ethyl-2,2-bis[4-(methylsulfonyl)phenyl]ethenyl}phenyl)oxy]ethyl}oxy)ethanol

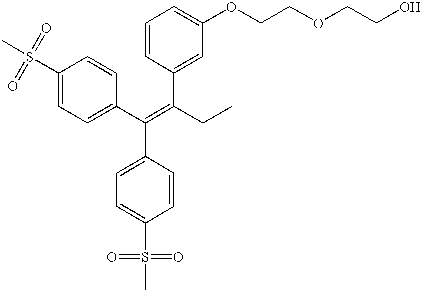

### Example 39 (#)

### (2E)-3-[5-[2,2-Bis(4-hydroxyphenyl)-1-propylethenyl]-2-(methyloxy)phenyl]-2-propenoic acid

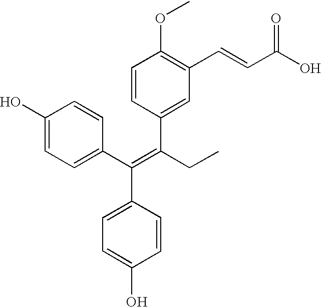

### Example 40 (#)

### (2E)-3-[5-[1-Butyl-2,2-bis(4-hydroxyphenyl)ethenyl]-2-(methyloxy)phenyl]-2-propenoic acid

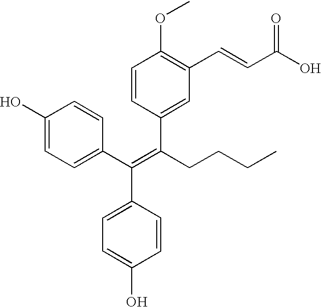

### Example 41 (#)

### (2E)-3-[5-[1-Butyl-2,2-bis(4-hydroxyphenyl)ethenyl]-2-(trifluoromethyl)phenyl]-2-propenoic acid

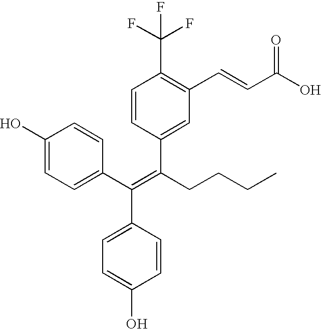

### Example 42 (#)

### 4,4′-{2-[3-[(2-Hydroxyethyl)oxy]-4-(methyloxy)phenyl]-1-pentene-1,1-diyl}diphenol

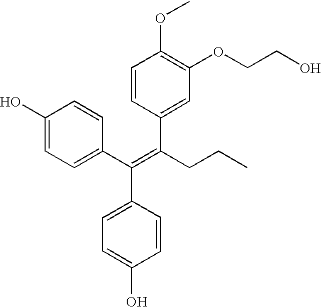

### Example 43 (#)

### 4,4′-{2-[3-{2-[(2-Hydroxyethyl)oxy]ethyl}-4-(methyloxy)phenyl]-1-pentene-1,1-diyl}diphenol

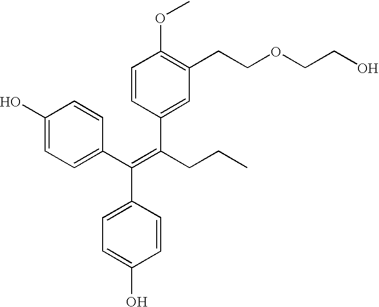

### Example 44 (#)

### 4,4′-{2-[3-[(2-Hydroxyethyl)oxy]-4-(methyloxy)phenyl]-1-hexene-1,1-diyl}diphenol

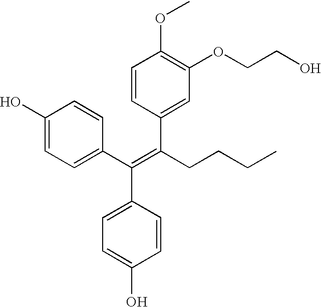

### Example 45 (#)

### 4,4′-{2-[3-{2-[(2-Hydroxyethyl)oxy]ethyl}oxy)-4-(methyloxy)phenyl]1-hexene-1,1-diyl}diphenol

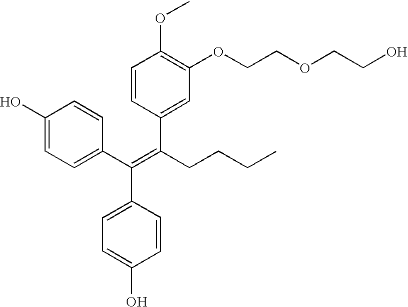

### Example 46 (#)

### 4-{[4-[2,2-Bis(4-hydroxyphenyl)-1-propylethenyl]-2-(methyloxy)phenyl]oxy}butanoic acid

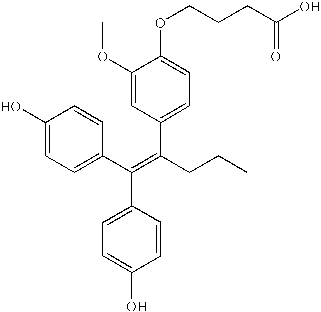

### Example 47 (#)

### 4-{[5-[1-Butyl-2,2-bis(4-hydroxyphenyl)ethenyl]-2-(methyloxy)phenyl]oxy}butanoic acid

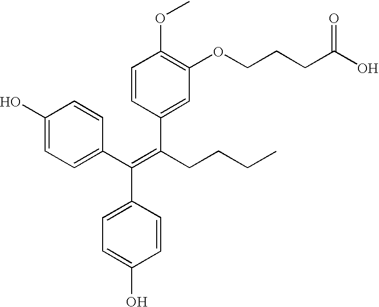

### Example 48(#)

### 3-[1-Butyl-2,2-bis(4-hydroxyphenyl)ethenyl]benzaldehyde oxime

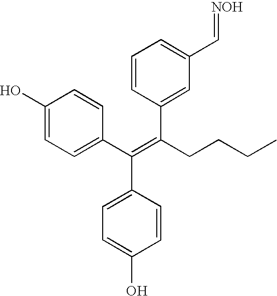

### Example 49 (#)

### 4,4′-{2-cyclohexyl-2-[3-({2-[(2-hydroxyethyl)oxy]ethyl}oxy)phenyl]-1,1-ethenediyl}diphenol

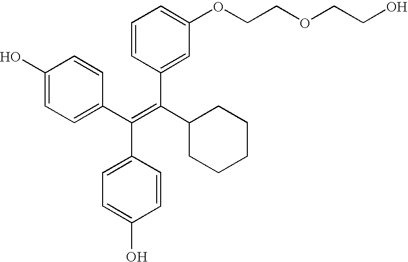

### Example 50 (#)

### 4,4′-{2-[3-({2-[(2-hydroxyethyl)oxy]ethyl}oxy)phenyl]-3,3-dimethyl-1-pentene-1,1-diyl}diphenol

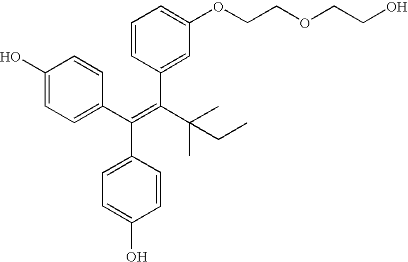

### Biological Data

### ER Alpha Fluorescence Polarisation Assays.

Assays were conducted using both full length and ligand binding domain protein.

Full length ER alpha—The assay was performed using a commercially available kit (P3029, Invitrogen, Carlsbad, Calif.). The assay was performed according to the manufacturer's protocol with minor amendments. Namely, 15 nM ERα and 1 nM Fluormone EL Red were dissolved and mixed in Complete ER Red Buffer. 10 μl of the mix was dispensed to each well of Greiner low volume plates—Black solid low volume 384-well plates—(Greiner—Product No. 784076), containing compounds within the concentration range of 10⁻⁵-10¹²M in dimethyl sulfoxide (DMSO). The plates were spun for 1 min at 200 g, covered to protect the reagents from light, and then incubated at room temperature for 2 hours. Plates were read on an Acquest, LJL Biosystems, Sunnyvale, Calif., using a 530-25 nm excitation and 580-10 nm emission interference filter and a 561 nm Dichroic mirror.

### Expression and Purification of ERα LBD

A cDNA sequence corresponding to residues 297 to 555 of human ERα (accession number NP_{—}000116.2) was cloned into a pET24vector (Novagen, San Diego, Calif.) with a N-terminal hexa-histidine tag. The plasmid was transformed into E. coli BL21-DE3 cells. Cells were grown at 23° C. for 18 hr, the temperature was lowered to 18° C. before addition of 250 μM IPTG. Cells were grown an additional 24 hr before harvesting. Cells were lysed in 50 mM TRIS pH 8.0/250 mM NaCl/2 M Urea and spun down. The supernatant was made 50 mM in imidazole and loaded onto a Ni-chelating sepharose column (Pharmicia) and eluted with a linear gradient of 50 to 500 mM Imidazole. Fractions containing ERα LBD were pooled and dialyzed against 50 mM TRIS pH 8.0/250 mM NaCl/5 mM DTT and 10% glycerol. Samples were aliquoted and frozen at −70° C.

The assay was performed by mixing 15 nM ER alpha LBD with 1 nM Fluormone-EL-Red (Invitrogen No. P3030) in assay buffer (Tris-HCl (50 mM; pH8), KCl, (500 mM), Dithiothreitol (1 mM), Ethylene diamine tetraacetic acid (1 mM), glycerol (10% v/v), 3 cholamidopropyl-dimethylammonio1-propanesulfonate-(2 mM), Sodium orthovanadate (1 mM—this was prepared as 100 mM stock by dissolving in distilled water and 2 successive rounds of adjusting pH to 10, boiling and cooling)). 10 μl of the mix was dispensed to each well of Greiner low volume plates—Black solid low volume 384-well plates—(Greiner, Longwood, Fla.—Product No. 784076), containing compounds within the concentration range of 10⁻⁵-10⁻¹²M in dimethyl sulfoxide (DMSO). The plates were spun for 1 min at 200 g, covered to protect the reagents from light, and then incubated at room temperature for 2 hours. Plates were read on the Acquest using a 530-25 nm excitation and 580-10 nm emission interference filter and a 561 nm Dichroic mirror.

### ER Beta Fluorescence Polarisation Assays.

Assays were conducted using both full length and ligand binding domain protein. Full length ER beta—The assay was performed using a commercially available kit (P3032, Invitrogen). The assay was performed according to the manufacturer's protocol with minor amendments. Namely, 30 nM ERβ and 1 nM Fluormone EL Red were dissolved and mixed in Complete ER Red Buffer. 10 μl of the mix was dispensed to each well of Greiner low volume plates—Black solid low volume 384-well plates—(784076, Greiner), containing compounds within the concentration range of 10⁻⁵-10⁻¹²M in dimethyl sulfoxide (DMSO). The plates were spun for 1 min at 200 g, covered to protect the reagents from light, and then incubated at room temperature for 2 hours. Plates were read on an Acquest (Acquest/Biosystems) using a 530-25 nm excitation and 580-10 nm emission interference filter and a 561 nm Dichroic mirror.

### Expression and Purification of ERβ LBD

A cDNA sequence corresponding to residues 257 to 530 of human ERβ (accession number NP_{—}001428.1) was cloned into a pRSETa (Novagen) vector with a N-terminal hexa-histidine tag. The plasmid was transformed into E. coli BL21-DE3 cells. The cells were grown at 23° C. for 18 hr, the temperature was lowered to 18° C. and then 250 μM of IPTG was added. Cells were grown an additional 24 hr before harvesting. Cells were lysed in 50 mM TRIS pH 8.0/250 mM NaCl and spun down. The supernatant wais made 50 mM in imidazole and loaded onto a Ni-chelating sepharose column (Amersham Pharmacia Biotech, Piscataway, N.J.) and eluted with a linear gradient of 50 to 500 mM Imidazole. Fractions containing ERβ LBD were pooled and diluted to 50 mM NaCl and loaded on a Q-sepharose column (Pharmacia) equilibrated with 50 mM TRIS pH 8.0150 mM NaCl/5 mM DTT and 10% glycerol. The ERβ, was eluted with a linear gradient from 50 mM to 500 mM NaCl. Fractions containing ERβ LBD were pooled and dialyzed against 50 mM TRIS pH 8.0/250 mM NaCl/S mM DTT and 10% glycerol. Samples were aliquoted and frozen at −70° C.

The assay was performed by mixing 30 nM ER beta LBD with 1 nM Fluormone-EL-Red (Invitrogen No. P3030) in assay buffer (Tris-HCl (50 mM; pH8), KCl, (500 mM), Dithiothreitol (1 mM), Ethylene diamine tetraacetic acid (1 mM), glycerol (10% v/v), 3 cholamidopropyl-dimethylammoniol-propanesulfonate—(2 mM), Sodium orthovanadate (1 mM—this was prepared as 100 mM stock by dissolving in distilled water and 2 successive rounds of adjusting pH to 10, boiling and cooling)). 10 μl of the mix was dispensed to each well of black solid low volume 384-well plates—(784076, Greiner), containing compounds within the concentration range of 10⁻⁵-10⁻¹²M in dimethyl sulfoxide (DMSO). The plates were spun for 1 min at 200 g, covered to protect the reagents from light, and then incubated at room temperature for 2 hours. Plates were read on the Acquest using a 530-25 nm excitation and 580-10 nm emission interference filter and a 561 nm Dichroic mirror.

### Data Analysis

All data was normalized to the mean of 16 high and 16 low control wells on each plate. A four parameter curve fit of the following form was then applied

$$y = \frac{a - d}{1 + {( x / c )}^{b}} + d$$

Where a is the minimum, b is the Hill slope, c is the IC₅₀ and d is the maximum. Data is presented as the mean pIC₅₀ with the standard deviation of the mean of n experiments.

The compounds of the Examples above exhibited pIC₅₀ values ranging from 6 to 8.5.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

What is claimed is:

1. A compound of Formula I 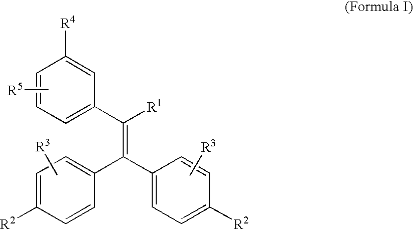
or a pharmaceutically acceptable salt or solvate thereof,
wherein
R¹ is selected from the group consisting of C₁-C₆ alkyl and C₁-C₆ haloalkyl;
each R is the same and selected from the group consisting of —OH, halogen, C₁-C₄ alkoxy, —S—R⁸, —SO—R⁸, —SO₂—R⁸, and —C≡N;
each R³ is the same and selected from hydrogen, —OH, C₁-C₆ alkyl, halogen, C₁-C₆ alkoxy, and C₁-C₆ haloalkyl;
R⁹ is independently selected from H and substituted or unsubstituted C₁-C₆ alkyl group;
R¹¹ and R¹² are independently selected from H, R^{a}H, R^{a}OH, and R^{a}OMe;
R^{a} and R^{b} are independently selected from C₁-C₆ alkylene, and,
R⁴ is selected from —COOH, —C≡N, —C≡C—R^{a}—OH, —C≡C—COO—R⁸, —C═NOH, —C═C—R⁶, O—R^{a}—R¹⁰, —N—R¹¹R¹², —N—SO₂—R⁸, —SOR⁸, —SO₂—
R⁸, and a saturated or unsaturated heterocycle containing 1 or 2 heteroatoms selected from N and O optionally substituted with one or more of C₁-C₆ alkyl, phenyl, and C₁-C₆ alkylphenyl;
R⁵ is selected from hydrogen, —OH, C₁-C₆ alkyl, halogen, C₁-C₆ alkoxy, and C₁-C₆ haloalkyl;
R⁶ is selected from R^{a}COOH, COO—R⁸—CONR⁸R⁹;
R⁸ is independently selected from H and substituted or unsubstituted C₁-C₆ alkyl group; and
R¹⁰ is selected from COOH, OH, O—R^{b}—OH, O—R^{b}—OCO—R⁸, and COO—R⁸; or,
R⁴ is selected from —COOH, —C≡N, —C═C—R⁶, —O—R—R¹, —SOR⁸, —SO₂—R⁸, and a saturated or unsaturated heterocycle containing 1 or 2 heteroatoms selected from N and O optionally substituted with one or more of C₁-C₆ alkyl, phenyl, and C₁-C₆ alkylphenyl;
R⁵ is selected from hydrogen, —OH, C₁-C₆ alkyl, halogen, C₁-C₆ alkoxy, and C₁-C₆ haloalkyl;
R⁶ is selected from —COOH, R^{a}—COOH, COO—R⁸, —CONR⁸R⁹;
R⁸ is selected from substituted or unsubstituted C₁-C₆ alkyl group; and
R¹⁰ is selected from COOH, OH, O—R^{b}—OH, O—R^{b}—OCO—R⁸, and COO—R⁸; or,
R⁴ is selected from —COOH, —C≡N, —C═C—R⁶, —OR^{a}—R¹⁰, —SOR⁸, —SO₂—R⁸, and a saturated or unsaturated heterocycle containing 1 or 2 heteroatoms selected from N and O optionally substituted with one or more of C₁-C₆ alkyl, phenyl, and C₁-C₆ alkylphenyl;
R⁵ is selected from hydrogen, —OH, halogen, and C₁-C₆ alkoxy;
R⁶ is selected from —COOH, R^{a}COOH, COO—R⁸;
R⁸ is an unsubstituted C₁-C₆ alkyl group; and
R¹⁰ is selected from COOH, OH, NH₂, NHR³, O—R—OH, O—R^{b}—OCO—R⁸, and COO—R⁸.

2. A compound of claim 1 or a pharmaceutically acceptable salt or solvate thereof, wherein
R¹ is selected from the group consisting of C₁-C₆ alkyl and C₁-C₆ haloalkyl;
each R² is the same and selected from the group consisting of —OH, halogen, C₁-C₄ alkoxy, —S—R⁸, —SO—R⁸, —SO₂—R⁸, and —C≡N;
each R³ is the same and selected from hydrogen, —OH, C₁-C₆ alkyl, halogen, C₁-C₆ alkoxy, and C₁-C₆ haloalkyl;
R⁴ is selected from —COOH, —C≡N, C≡C—R^{a}—OH, —C═C—COO—R⁸, —C═NOH, —C═C—R⁶, —O—R^{a}—R¹⁰, —N—R¹¹R¹², —N—SO₂—R⁸, —SO₂—R⁸, and a saturated or unsaturated heterocycle containing 1 or 2 heteroatoms selected from N and O optionally substituted with one or more of C₁-C₆ alkyl, phenyl, and C₁-C₆ alkylphenyl;
R⁵ is selected from hydrogen, —OH, C₁-C₆ alkyl, halogen, C₁-C₆ alkoxy, and C₁-C₆ haloalkyl;
R⁶ is selected from R^{a}—COOH, COO—R⁸, —CONR⁸R⁹;
R⁸ and R⁹ are independently selected from H and substituted or unsubstituted C₁-C₆ alkyl group; and
R¹⁰ is selected from COOH, OH, O—R^{b}—OH, O—R^{b}—OCO—R⁸, and COO—R⁸
R¹¹ and R¹² are independently selected from H, R^{a}H, R^{a}OH, and R^{a}OMe; and
R^{a} and R^{b} are independently selected from C₁-C₆ alkylene.

3. The compound of claim 2 or a pharmaceutically acceptable salt or solvate thereof, wherein R⁴ is selected from —COOH, —C≡N, —C≡C—R^{a}OH, —C≡C—COO—R⁸, —C≡NOH, —C═C—R⁶, —O—R^{a}—R¹⁰, and —N—R¹¹R¹².

4. The compound of claim 3 or a pharmaceutically acceptable salt or solvate thereof, wherein R¹ is a C₁₋₆ alkyl.

5. The compound of claim 4 or a pharmaceutically acceptable salt or solvate thereof wherein R¹ is ethyl.

6. The compound of claim 2 or a pharmaceutically acceptable salt or solvate thereof, wherein R³ is hydrogen.

7. The compound of claim 2 or a pharmaceutically acceptable salt or solvate thereof, wherein each R² is —OH.

8. The compound of claim 2 or a pharmaceutically acceptable salt or solvate thereof, wherein R⁴ is selected from —C═C—COOH, —COOH, and —O(CH₂)₂O(CH₂)₂OH.

9. (canceled)

10. (canceled)

11. A compound of claim 1
or a pharmaceutically acceptable salt or solvate thereof, wherein
R¹ is selected from the group consisting of C₁-C₆ alkyl and C₁-C₆ haloalkyl;
each R² is the same and selected from the group consisting of —OH, halogen, C₁-C₄ alkoxy, —S—R⁸, —SO—R⁸, —SO₂—R⁸, and —C≡N;
each R³ is the same and selected from hydrogen, —OH, C₁-C₆ alkyl, halogen, C₁-C₆ alkoxy, and C₁-C₆ haloalkyl;
R⁴ is selected from —COOH, —C≡N, —C═C—R⁶, —O—R^{a}—R¹⁰, —SO₂—R⁸, and a saturated or unsaturated heterocycle containing 1 or 2 heteroatoms selected from N and O optionally substituted with one or more of C₁-C₆ alkyl, phenyl, and C₁-C₆ alkylphenyl;
R⁵ is selected from hydrogen, —OH, C₁-C₆ alkyl, halogen, C₁-C₆ alkoxy, and C₁-C₆ haloalkyl;
R⁶ is selected from —COOH, R^{a}—COOH, COO—R⁸, —CONR⁸R⁹;
R^{a} and R^{b} are independently selected from C₁-C₆ alkylene;
R⁸ is selected from substituted or unsubstituted C₁-C₆ alkyl group; and
R¹⁰ is selected from COOH, OH, O—R^{b}—OH, O—R^{b}—OCO—R⁸, and COO—R⁸.

12. The compound of claim 11 or a pharmaceutically acceptable salt or solvate thereof, wherein R⁴ is selected from —COOH, —C≡N, —C═C—R⁶, and —O—R—R¹⁰, and —SO₂—R⁸.

13. The compound of claim 11 or a pharmaceutically acceptable salt or solvate thereof, wherein R¹ is a C₁₋₆ alkyl.

14. The compound of claim 13 or a pharmaceutically acceptable salt or solvate thereof wherein R¹ is ethyl.

15. The compound of claim 11 or a Pharmaceutically acceptable salt or solvate thereof, wherein R³ is hydrogen.

16. The compound of claim 11 or a pharmaceutically acceptable salt or solvate thereof, wherein each R² is —OH.

17. The compound of claim 11 or a
pharmaceutically acceptable salt or solvate thereof, wherein R⁴ is selected from —COOH, —C═C—COOH, and —O(CH₂)₂O(CH₂)₂OH.

18. (canceled)

19. A compound of claim 1
or a pharmaceutically acceptable salt or solvate thereof, wherein
R¹ is selected from the group consisting of C₁-C₆ alkyl and C₁-C₆ haloalkyl;
each R² is the same and selected from the group consisting of —OH, halogen, C₁-C₄ alkoxy, —S—R⁸, —SO—R⁸, —SO₂—R⁸, and —C≡N
each R³ is the same and selected from hydrogen, —OH, C₁-C₆ alkyl, halogen, C₁-C₆ alkoxy, and C₁-C₆ haloalkyl;
R⁴ is selected from —COOH, —C≡N, —C═C—R⁶—R^{a}—R¹⁰, —SO₂—R⁸, and a saturated or unsaturated heterocycle containing 1 or 2 heteroatoms selected from N and O optionally substituted with one or more of C₁-C₆ alkyl, phenyl, and C₁-C₆ alkylphenyl;
R⁵ is selected from hydrogen, —OH, halogen, and C₁-C₆ alkoxy;
R⁶ is selected from —COOH, R^{a}—COOH, COO—R⁸;
R^{a} and R^{b} are independently selected from C₁-C₆ alkylene;
R⁸ is an unsubstituted C₁-C₆ alkyl group; and
R¹⁰ is selected from COOH, OH, NH₂, NHR⁸, O—R^{b}—OH, O—R^{b}—OCO—R⁸, and COO—R⁸.

20. A pharmaceutical composition comprising the compound or a pharmaceutically acceptable salt or solvate thereof according to claim 1 and a pharmaceutically acceptable carrier, diluent or excipient.

21. (canceled)

22. (canceled)

23. (canceled)

24. (canceled)

25. A pharmaceutical composition comprising the compound of claim 1 or a pharmaceutically acceptable salt or solvate thereof in combination with other therapeutic agents selected from a bone building agent, anti-bone resorption agent, growth promoting agents, growth hormone secretagogues, growth hormone releasing factor and its analogs, growth hormone and its analogs, somatomedins, alpha-ardenergic agonists, serotonin 5-HT_{D} agonists, selective serotonin reuptake inhibitors, agents that inhibit somatostatin or its release, 5-α-reductase inhibitors, aromatase inhibitors, GnRH inhibitors, parathyroid hormone, bisphosphonates, estrogen, testosterone, SERMs, progesterone receptor agonists, and other modulators of nuclear hormone receptors.

26. (canceled)

27. (canceled)

28. A method of treatment for a condition or disorder affected by selective estrogen regulator modulation in a mammal in need thereof comprising administering a therapeutically effective amount of the compound of claim 1 or a pharmaceutically acceptable salt or solvate thereof
wherein the condition or disorder is selected from osteoporosis, bone demineralization, reduced bone mass, density, or growth, osteoarthritis, acceleration of bone fracture repair and healing, acceleration of healing in joint replacement, periodontal disease, acceleration of tooth repair or growth, Paget's disease, osteochondrodysplasias, muscle wasting, the maintenance and enhancement of muscle strength and function, frailty or age-related functional decline (“ARFD”), sarcopenia, chronic fatigue syndrome, chronic myaligia, acute fatigue syndrome, acceleration of wound healing, maintenance of sensory function, chronic liver disease, AIDS, weightlessness, burn and trauma recovery, thrombocytopenia, short bowel syndrome, irritable bowel syndrome, inflammatory bowel disease, Crohn's disease and ulcerative colitis, obesity, eating disorders including anorexia associated with cachexia or aging, hypercortisolism and Cushing's syndrome, cardiovascular disease or cardiac dysfunction, congestive heart failure, high blood pressure, breast cancer, malignant tumor cells containing the androgen receptor including breast, brain, skin, ovary, bladder, lymphatic, liver, kidney, uterine, pancreas, endometrium, lung, colon, and prostate, prostatic hyperplasia, hirsutism, acne, seborrhea, androgenic alopecia, anemia, hyperpilosity, adenomas and neoplasis of the prostate, hyperinsulinemia, insulin resistance, diabetes, syndrome X, dyslipidemia, urinary incontinence, artherosclerosis, libido enhancement, sexual dysfunction, depression, depressive symptoms, nervousness, irritability, stress, reduced mental energy and low self-esteem, improvement of cognitive function, endometriosis, polycystic ovary syndrome, counteracting preeclampsia, premenstral syndrome, contraception, uterine fibroid disease, and/or aortic smooth muscle cell proliferation, vaginal dryness, pruritis, dyspareunia, dysuria, frequent urination, urinary tract infections, hypercholesterolemia, hyperlipidemia, peripheral vascular disease, restenosis, vasospasm, vascular wall damage due to immune responses, Alzheimer's disease, bone disease, aging, inflammation, rheumatoid arthritis, respiratory disease, emphysema, reperfusion injury, viral hepatitis, tuberculosis, psoriasis, systemic lupus erythematosus, amyotrophic lateral sclerosis, stroke, CNS trauma, dementia, neurodegeneration, breast pain and dysmenorrhea, menopausal or postmenopausal disorders, vasomotor symptoms, urogenital or vulvar vaginal atrophy, atrophic vaginitis, female sexual dysfunction, for enhancing libido, for the treatment of hypoactive sexual disorder, sexual arousal disorder, for increasing the frequency and intensity of orgasms, vaginismus, osteopenia, endometriosis, BPH (benign prostatic hypertrophy), dysmenorrhea, autoimmune diseases, Hashimoto's thyroiditis, SLE (systemic lupus erythematosus), myasthenia gravis, or reperfusion damage of ischemic myocardium.

29. The method of claim 28 wherein the disorder or condition is selected from menopausal or postmenopausal disorders, vasomotor symptoms, urogenital or vulvar vaginal atrophy, atrophic vaginitis, endometriosis, female sexual dysfunction, breast cancer, depressive symptoms, diabetes, bone demineralization, and osteoporosis.

30. A pharmaceutical composition comprising the compound or a pharmaceutically acceptable salt or solvate thereof according to claim 2 and a pharmaceutically acceptable carrier, diluent or excipient.

31. A pharmaceutical composition comprising the compound or a pharmaceutically acceptable salt or solvate thereof according to claim 11 and a pharmaceutically acceptable carrier, diluent or excipient.

32. A pharmaceutical composition comprising the compound or a pharmaceutically acceptable salt or solvate thereof according to claim 19 and a pharmaceutically acceptable carrier, diluent or excipient.
